(19) United States
(12) Patent Application Publication
(10) Pub. No.: US 2024/0369576 A1
(43) Pub. Date: Nov. 7, 2024

(54) ASSAYS FOR ALPHA-DYSTROGLYCAN GLYCOSYLATION AND METHODS FOR USING SAME

(71) Applicants: ML Bio Solutions Inc., Palo Alto, CA (US); BridgeBio Services Inc., Palo Alto, CA (US)

(72) Inventors: Hector M. RODRIGUEZ, San Bruno, CA (US); Thulashitha RAJASINGHAM, Foster City, CA (US); Andreas H. Betz, Mountain View, CA (US)

(21) Appl. No.: 18/605,744

(22) Filed: Mar. 14, 2024

Related U.S. Application Data
(60) Provisional application No. 63/490,216, filed on Mar. 14, 2023.

(51) Int. Cl. G01N 33/68; G01N 33/543

(52) U.S. Cl. CPC G01N 33/6893; G01N 33/5436; G01N 33/6854; G01N 2333/4728; G01N 2440/38; G01N 2800/52

(57) ABSTRACT

Provided are methods of determining an amount of a glycosylated form of alpha-dystroglycan (αDG) in a sample. In some embodiments, the methods provided herein may be useful in the diagnosis, evaluation, and/or monitoring of a subject having a dystroglycanopathy, such as Walker-Warburg Syndrome, Muscle Eye Brain Disease, Fukuyama Congenital Muscular Dystrophy (CMD), or a Limb Girdle Muscular Dystrophy.

### CROSS-REFERENCE TO RELATED APPLICATIONS

The present application claims priority to U.S. Provisional Application 63/490,216, filed Mar. 14, 2023; the contents of which are incorporated herein by reference in their entireties.

### BACKGROUND

In healthy muscle cells, the sugar chain on the protein of alpha-dystroglycan (αDG) contains tandem structures of ribitol-phosphate, a pentose alcohol. The genes fukutin (FKTN), fukutin-related protein (FKRP), and isoprenoid synthase domain-containing protein (ISPD) encode essential enzymes for the synthesis of this structure. ISPD metabolically converts ribitol-5-phosphate into CDP-ribitol, a substrate for fukutin and FKRP. Subsequently, fukutin transfers a first ribitol-phosphate onto sugar chains of αDG followed by FKRP that transfers a subsequent ribitol-phosphate. Abnormal glycosylation of αDG is associated with a range of neurological and physical impairments. A variety of dystroglycanopathies including Walker-Warburg Syndrome, Muscle Eye Brain Disease, Fukuyama Congenital Muscular Dystrophy (CMD), cobblestone brain malformation or cobblestone formation of neuronal tissue (COB or lissencephaly type 2), and Limb Girdle Muscular Dystrophies have been identified and are generally characterized by different genetic mutations affecting different enzymes involved in the glycosylation of dystroglycan. Limb-Girdle Muscular Dystrophy type 2i, also known as LGMD R9 (LGMD2I/R9), is a dystroglycanopathy caused by partial loss of function mutations in the FKRP gene.

LGMD2I/R9 is an autosomal recessive disease of striated muscle caused by mutations in FKRP. The FKRP enzyme performs a critical step in the glycosylation of αDG. The heavily glycosylated αDG is a component of the dystrophin-glycoprotein complex that anchors the intracellular cytoskeleton of muscle cells to the extracellular matrix through interactions of the matriglycan with laminin. In the context of LGMD2I/R9, impaired FKRP enzyme activity leads to the formation of dysfunctional hypoglycosylated αDG, resulting in chronic myocyte injury and muscular dystrophy. This makes muscle cells susceptible to contraction-induced injury that results in inflammation, fibrosis, and fatty infiltrate leading to muscle wasting and impaired function.

Ribitol is currently being investigated in clinical trials as a potential therapy for use in the treatment of LGMD2I/R9. Without wishing to be bound by theory, ribitol may increase the intracellular concentration of CDP-ribitol, enabling a deficient FKRP to be more active and allowing αDG to be primed for further glycosylation. Ribitol may thus influence the glycosylation state of αDG.

Heng et al. discloses methods of detecting αDG in human urine using a sandwich ELISA technique (Heng et al., J. Biomolec Screen, 4(21); 2015). However, simultaneous detection of αDG and glycosylated αDG is needed for rapid and consistent quantification of the extent of αDG glycosylation to keep pace with the dynamic nature of this biochemical process. Accordingly, there remains an unmet need for methods of simultaneous detection of αDG and glycosylated αDG for dystroglycanopathy patient biopsies.

### SUMMARY

The present disclosure provides methods for treating dystroglycanopathies and evaluating the same. The present disclosure also provides methods of assessing a glycosylation state of alpha-dystroglycan (αDG). The methods provided herein may involve treatment of dystroglycanopathies (e.g., Walker-Warburg Syndrome, Muscle Eye Brain Disease, Fukuyama Congenital Muscular Dystrophy (CMD), and Limb Girdle Muscular Dystrophies such as limb girdle muscular dystrophy type 2i (LGMD2I/R9)) and evaluation of the same via, e.g., assessment of αDG. Methods of assessing αDG may involve measuring both total αDG and glycosylated αDG using, e.g., a multiplexed Western Blot approach. Such methods may comprise assessment of biopsies from subjects taken prior to, during, and/or following treatment of the subjects with ribitol. The methods provided herein may also be useful to evaluate the impact of therapeutics that seek to restore the glycosylation of αDG and may assist in guiding dose selection.

Accordingly, in an aspect, the present disclosure provides a method comprising: a) determining a difference between a first amount of a glycosylated form of alpha-dystroglycan (αDG) in a first sample taken from a subject undergoing treatment for a dystroglycanopathy at a first time and a second amount of αDG in a second sample taken from the subject at a second time; and b) based at least in part on a), determining that the treatment should be continued, ii) determining that the treatment should be discontinued, or iii) determining that the treatment should be adjusted.

In an aspect, the present disclosure relates to a method of evaluating a subject undergoing treatment for a dystroglycanopathy, comprising: a) providing a first sample containing a first amount of a glycosylated form of alpha-dystroglycan (αDG) from a subject, wherein the first sample is taken from the subject at a first time; b) providing a second sample containing a second amount of a glycosylated form of αDG from the subject, wherein the second sample is taken from the subject at a second time later than the first time; c) identifying (e.g., determining) the first amount in the first sample; d) identifying (e.g., determining) the second amount in the second sample; and e) determining a difference between the first amount and the second amount.

In some embodiments, c) and d) are performed at the same time. In some embodiments, c) and d) are performed at different times.

In some embodiments, the method further comprises f) based at least in part on the difference determined in e), i) determining that the treatment should be continued, ii) determining that the treatment should be discontinued, or iii) determining that the treatment should be adjusted.

In some embodiments, iii) comprises determining that a dose of the therapeutic agent should be increased. In some embodiments, iii) comprises determining that a dose of the therapeutic agent should be decreased.

In some embodiments, the first time is prior to commencement of the treatment for the dystroglycanopathy. In some embodiments, the second time is during or after the treatment of the dystroglycanopathy. In some embodiments, the second time is at least about 3 months after the first time. In some embodiments, the second time is at least about 6 months after the first time. In some embodiments, the second time is at least about 9 months after the first time. In some embodiments, the second time is at least about 12 months after the first time.

In some embodiments, the first sample and the second sample are tissue biopsy samples. In some embodiments, the first sample and the second sample are tibialis anterior (TA) samples.

In some embodiments, determining the difference between the first amount and the second amount comprises performing a Western blotting analysis. In some embodiments, the first amount and the second amount are determined by integrating a signal over a range of interest and interpolating the signal to a standard curve. In some embodiments, the interpolation is performing using regression to a quadratic equation. In some embodiments, the standard curve is based on amounts of glycosylated αDG measured for healthy subjects not undergoing treatment for a dystroglycanopathy. In some embodiments, the Western blotting analysis contacting the sample with one or more antibodies comprises contacting the sample with one or more antibodies. In some embodiments, an antibody of the one or more antibodies is used to determine an amount of the glycosylated form of αDG having a molecular weight in the range of interest of between about 125 kiloDaltons (kDa) and about 260 kDa. In some embodiments, the one or more antibodies are selected from AF6868 alpha-dystroglycan, IIH6C4 alpha-dystroglycan, IR800CW Mouse Anti-Sheep, and IR680 Goat Anti-Mouse antibodies. In some embodiments, determining the first amount and the second amount comprises detection of a fluorescent signal at about 700 nanometers (nm) and/or 800 nm.

In some embodiments, the dystroglycanopathy is selected from Walker-Warburg Syndrome, Muscle Eye Brain Disease, Fukuyama Congenital Muscular Dystrophy (CMD), or a Limb Girdle Muscular Dystrophy. In some embodiments, the dystroglycanopathy is limb girdle muscular dystrophy type 2i (LGMD2I/R9), LGMD type 2m (LGMD2m), or LGMD type 2u (LGMD2u). In some embodiments, the dystroglycanopathy is limb girdle muscular dystrophy type 2i (LGMD2I/R9).

In some embodiments, the treatment for the dystroglycanopathy comprises administration of a therapeutic agent. In some embodiments, the therapeutic agent is ribitol or a pharmaceutical composition comprising the same. In some embodiments, the treatment comprises administering an effective amount of ribitol to the subject. In some embodiments, the treatment comprises administering at least 0.5 grams of ribitol per day (g/day), at least 1 g/day, at least 2 g/day, at least 3 g/day, at least 4 g/day, at least 5 g/day, at least 7.5 g/day, at least 10 g/day, at least 12.5 g/day, at least 15 g/day, at least 20 g/day, at least 25 g/day, at least 30 g/day, at least 35 g/day, at least 40 g/day, at least 45 g/day, at least 50 g/day, at least 55 g/day, or at least 60 g/day to the subject. In some embodiments, the treatment comprises administering 0.5 grams of ribitol per day (g/day), 1 g/day, 1.5 g/day, 2 g/day, 3 g/day, 4 g/day, 5 g/day, 6 g/day, 7.5 g/day, 10 g/day, 12 g/day, 12.5 g/day, 15 g/day, 20 g/day, 25 g/day, 30 g/day, 35 g/day, 40 g/day, 45 g/day, 50 g/day, 55 g/day, or 60 g/day. In some embodiments, the treatment comprises administering ribitol at a dose effective to achieve an area under the concentration-time curve (AUC0-24) steady state level of between 100 (μg·h)/mL and 8000 (μg·h)/m.

In some embodiments, the method further comprises evaluating the subject using the North Star Assessment for Limb Girdle Type Muscular Dystrophies (NSAD), Performance of upper limb test 2.0 (PUL2.0), 10-meter walk test (10MWT), 100-meter timed test (100MTT), forced vital capacity (FVC) assessment, serum creatine kinase (CK) levels, or a combination thereof.

In some embodiments, the second amount is greater than the first amount. In some embodiments, the second amount is at least about 10%, about 20%, about 30%, about 40%, about 50%, about 60%, about 70%, about 80%, about 90%, about 100%, about 120%, about 140%, about 160%, about 180%, about 200%, about 250%, about 300%, about 350%, about 400%, about 450%, about 500%, or greater than the first amount. In some embodiments, the second amount is at least double the first amount.

In some embodiments, the method further comprises determining a first amount of total αDG in the first sample and a second amount of total αDG in the second sample. In some embodiments, the method further comprises determining (i) a first ratio of the glycosylated form of αDG to the total αDG in the first sample and (ii) a second ratio of the glycosylated form of αDG to the total αDG in the second sample.

Additional embodiments will be apparent from the disclosure provided herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram of the model for ribitol-induced functional glycosylation of αDG in FKRP mutant cells. ?=a mechanism is not fully understood; “*”=the first ribitol-5-phospate on the Core M3 of αDG is transferred by fukutin using CDP-ribitol as the donor substrate; CTP=Cytidine Triphosphate; D-Glucuronic acid (GlcA); Xylitol (Xyl); N-Acetyl-D-galactosamine (GalNAc); N-Acetyl-D-glucosamine (GlcNAc); D-Mannose (Man) (Source: Cataldi et al. 2018)

FIG. 2A is a graph depicting the linearity of each detection channel as a function of lysate concentration for serially diluted gastrocnemius tissue lysate. The solid line represents the curve fit by linear regression, with one channel corresponding to the core protein and the other to the glycosylated protein.

FIG. 2B is an image of a Western Blot probed for total α-dystroglycan (800 nanometers (nm)-green) and glycosylated α-dystroglycan (700 nm—red) by multiplexed LI-COR and displays the merge of both detection channels (yellow) with regions of interest for αDG (˜50-260 kDa) and a putative N-terminal fragment (˜27 kDa) indicated with yellow boxes. Red * denotes a sample that appeared to be predominantly adipose rather than muscle during tissue processing.

FIG. 2C is a graph depicting the ratio of glycosylated αDG to total αDG protein determined using intensities from the detection channels shown in FIG. 2B and plotted for each muscle type. The plot shows the mean and the standard deviation of the ratios.

FIG. 3A is an image of a Western Blot probed for total α-dystroglycan (800 nm—green) and glycosylated α-dystroglycan (700 nm—red) by multiplexed LI-COR and displays the merge of both detection channels (yellow) in healthy and mutant (P488L and L276I) mouse heart and gastrocnemius tissues.

FIG. 3B is an image of a Western Blot probed for total α-dystroglycan (800 nm—green) and glycosylated α-dystroglycan (700 nm—red) by multiplexed LI-COR and displays the merge of both detection channels (yellow) in healthy and mutant (P488L and L276I) mouse diaphragm and tibialis anterior (TA) tissues.

FIG. 3C is a graph depicting the ratio of total αDG to total protein determined using intensities from the detection channels shown in FIGS. 3A and 3B and plotted for each muscle type. All mutants were considered as a single dataset for the statistical analysis. The plot shows the mean and the standard deviation of the ratios. Statistical significance was determined by t-test: *: p=0.05-0.01; ***: p=0.001-0.0001; ****: p<0.0001.

FIG. 3D is a graph depicting the ratio of glycosylated αDG to total αDG determined using intensities from the detection channels shown in FIGS. 3A and 3B and plotted for each muscle type. All mutants were considered as a single dataset for the statistical analysis. The plot shows the mean and the standard deviation of the ratios. Statistical significance was determined by t-test: *: p=0.05-0.01; ***: p=0.001-0.0001; ****: p<0.0001.

FIG. 4 is a graph depicting the ratio of glycosylated αDG to total αDG assessed in gastrocnemius muscle tissue from a healthy human donor (Healthy Human) and a wild type mouse (WT-Mouse), TA muscle tissue from a L276I FKRP mutant mouse model (L276I Mouse) and a P448L mutant mouse model (P448L Mouse), and three LGMD2I patients containing a L276I FRKP mutation (two homozygous: [P1 L276I and P3 L276I] and one compound heterozygous: [P2 L276I]).

FIG. 5 is a graph depicting the ratio of glycosylated αDG to total αDG assessed in human LGMD2I patients with mutation L276I. The points on the plot represent the average of triplicate analysis for each patient. Shown are the mean and standard deviation for the healthy controls, L276I FKRP homozygous and compound heterozygous patients. The asterisks denote statistical significance. (HOM p=0.0125, HET p=0.0049 relative to control).

FIG. 6A is an image of a Western Blot probed for total α-dystroglycan (800 nm—green) and glycosylated α-dystroglycan (700 nm—red) by multiplexed LI-COR and displays the merge of both detection channels (yellow) with regions of interest for αDG (˜50-250 kDa) indicated with yellow boxes. The samples included healthy heart muscle tissue, healthy gastrocnemius muscle tissue, and P448L mutant FKRP heart and gastrocnemius tissues that were untreated or treated with 5% Ribitol, and an untreated C57 control. Healthy heart and gastrocnemius samples were run at 20, 10, and 5 micrograms of protein per lane as a loading control; mutant and C57 control lysate samples were run with 20 micrograms of protein per lane.

FIG. 6B is a graph depicting total αDG signal in untreated, 5% Ribitol treated, and C57 control samples was quantified using the regions of interest for αDG (Molecular weight ˜50-250 kDa) indicated with yellow boxes in FIG. 6A. All data points were normalized to the C57 untreated control.

FIG. 6C is a graph depicting the ratio of glycosylated αDG to total αDG signal in untreated, 5% Ribitol treated, and C57 control samples were quantified using the regions of interest for αDG (˜50-250 kDa) indicated with yellow boxes in FIG. 6A. All data points were normalized to the C57 untreated control.

FIG. 7 schematically illustrates the workflow for the Western blot assay of Example 2.

FIG. 8 shows a Western blot merged image corresponding to the assay of Example 2 showing the specificity of both primary antibodies, as well as staining of a recombinant human αDG (rh DG).

FIG. 9A shows a Western blot merged image corresponding to the assay of Example 2 showing the dilution series of TA lysate starting from 60 μg to 1 μg. The 3 mg TA tissue preparation had insufficient material to include a 60 μg point. FIG. 9B is a linear plot of the 700 and 800 nm signals from the analysis for the Western blot (depicting total protein).

FIGS. 10A-10B show Western blot merged examples containing patient samples as described in Example 2.

FIG. 11A shows the normalized ratio determined by interpolation of signal intensities and using the raw signal intensities from integration (700 nm/800 nm) for the assay of Example 2. FIG. 11B shows the interpolated amounts (on a log scale) of total αDG (αDG) and glycosylated αDG (αDG-gly) for the homozygous and compound heterozygous patients relative to healthy tissue. Heterozygous *CP denotes compound pathogenic heterozygous mutation.

FIG. 12 schematically illustrates a workflow for the Western Blot method of Example 3.

FIGS. 13A-13B show that treatment with ribitol dramatically increases αDG glycosylation in LGMD2I/R9 muscle biopsies, as described in Example 3.

FIGS. 14A-14B show that LGMD2I/R9 patients have less functional, glycosylated αDG relative to healthy subjects, as described in Example 3.

FIG. 15 shows that glycosylated αDG levels remain stable over time, as described in Example 3.

FIG. 16A-16B schematically illustrate the LGMD2I/R9 disease mechanism (FIG. 16A) and therapeutic approach (FIG. 16B) described in Example 4.

FIG. 17 schematically illustrates the details of the clinical study of Example 4.

FIGS. 18A-18B demonstrate the effects of ribitol treatment on glycosylated αDG levels observed in the study described in Example 4 compared to those observed in the natural history described in Example 3.

FIG. 19 show the maximum change in glycosylated αDG relative to baseline observed for individual patients for the clinical study of Example 4.

FIG. 20 shows the change in serum creatine kinase, a biomarker of muscle damage, after 15 months of treatment with ribitol as described in Example 4.

FIGS. 21A-21C show the improvements in ambulatory and clinical measures including change from baseline in 10MWT (FIG. 21A), change from baseline in 100MTT (FIG. 21B), and change from baseline in NSAD (FIG. 21C) after 15 months of treatment with ribitol as described in Example 4.

FIG. 22 schematically illustrates a planned Phase 3 clinical study.

FIGS. 23A-23C show a Western blot example with calibration curve and positive control, as described in Example 5. FIG. 23A shows the individual channels at 700 and 800 nm in the molecular weight range of 125-260 kDa for a calibration curve and QC control generated from donor TA. FIG. 23B shows the merged signals for the same blot, and FIG. 23C shows the two signal intensities versus protein load.

FIGS. 24A-24D shows a dot blot analysis for testing cross-reactivity, as described in Example 5. FIG. 24A shows 700 nm signal intensity from the IIH6C4 antibody, which binds to the unique O-mannosyl glycoepitope in the matriglycan chain of αDG. FIG. 24B shows a table summarizing the conditions explored to assess cross-reactivity. FIGS. 24C-24D are plots of the raw signal for IIH6C4 and AF6868, respectively. No cross-reactivity is indicated by the absence of signal at varying concentrations, whereas a positive response is indicated by concentration dependent increase in RFU.

FIGS. 25A-25D show inter-donor variability in normal human TA samples, as described in Example 5. FIG. 25A shows a table describing the demographics of the normal (non-affected) donors and the ID used on the WB. FIG. 25B shows a WB image captured by Li-COR system shown with both 700 nm and 800 nm channels merged. FIG. 25C shows a plot of the 700 nm signal for glycosylated αDG at total protein concentrations of 10, 20, 30, and 40 μg. TA1 is plotted separately to show the signal intensities are comparable to the average signal of all six donors. FIG. 25D shows a table summarizing the average signal from 6 TAs at different concentrations as well as the average signal from TA1.

FIG. 26 shows fluorescence signals at 700 nm and 800 nm from the standards from seven different Western blots, as described in Example 5. The Y axis depicts the raw fluorescence units (RFU), and the X axis depicts the amount of total protein in μg. RFU=raw fluorescence units. The fluorescence signals of calibration curves from seven WB are plotted versus the amount of total protein loaded. The solid line results from regression to a quadratic equation and the dashed line from that to a linear equation. The individual data points are summarized in boxplots with the middle line representing the median. The equations for the linear regression are RFU=−859(−451)+1190(705) amount and for the quadratic equation RFU=−398(−248)+1012(627) amount (μg)+7.2(3.2)×amount (μg)². The values in parentheses represent the regression results for the 800 nm channel.

FIGS. 27A-27B show signal trend in blank wells, as described in Example 5. FIG. 27A shows a Levi-Jenning's plot showing the signal intensities (solid lines) for blanks across 7 blots. Each blot is N=3 wells except for Blot 7 which was 1 well. The red dotted line is 3× standard deviation of the 700 nm background signal. The green dotted line is the 3× standard deviation of the 800 nm channel.

FIG. 27B shows a table summarizing data from 7 blots.

FIGS. 28A-28B show assay sensitivity in a fixed 100 μg tissue matrix, as described in Example 5. MWM=molecular weight marker. Blot has been split to show only relevant data. 700 nm and 800 nm channels are merged for the same blot in FIG. 28A. On the right of FIG. 28A is a plot of the 700 nm signal intensities, which correspond to matriglycan and the 800 nm signals corresponding to core αDG, versus the total amount of spiked in protein. The solid lines correspond to a fit with a quadratic equation. FIG. 28B shows a table that describes the coefficient of determination (R²) for both the spiked in and neat calibration standards.

FIGS. 29A-29B show results from inter- and intra-assay precision, as described in Example 5. FIG. 29A shows intra-assay variability based in absolute signal for Negative Validation Control (NC), High Positive Control (HPC) and Low Positive Control (LPC). FIG. 29B shows a table describing the inter assay variability based on interpolated percent nominal values of quality controls for HPC which was tested at 10 μg total protein and LPC which was tested at 5 μg total protein.

FIGS. 30A-30B show a blot from a natural history study in patients with the common homozygous L276I mutation and other FKRP genotypes, as described in Example 5. “Hom” indicates patients homozygous for L276I mutation; “Other” indicates patients with compound heterozygous mutations with either L2761/Other or Other/Other FKRP mutation. FIG. 30A is the same blot when imaged under separate 700 nm and 800 nm channels. FIG. 30B is the blot with merged channels. As described previously, a perfect overlap of channels results in yellow bands. Fully glycosylated protein is expected to migrate at 160 Da molecular weight. Decreases in glycosylated αDG (faint yellow band) at the 160 kDa molecular weight region and reduced molecular weight of αDG core for patient 2 with other FKRP mutation, which appears as multiple bands spanning the ROI of 70 to 160 kDa, suggests hypo glycosylation of αDG.

FIG. 31 shows relative levels of αDG in skeletal biopsies of patients with LGMD2I/R9, as described in Example 5. The figure presents relative levels of αDG from patient biopsies in FIGS. 30A-30B. The relative percent levels of αDG were determined from the 700 nm signal (for glycosylated αDG) and 800 nm signal (for core αDG) were interpolated to the calibration curve. The resulting relative μg amount was normalized by the total protein loaded.

FIG. 32 shows a validated Western blot assay effectively evaluates change from baseline in glycosylated αDG as early as 3 months after commencement of treatment with ribitol (BBP-418).

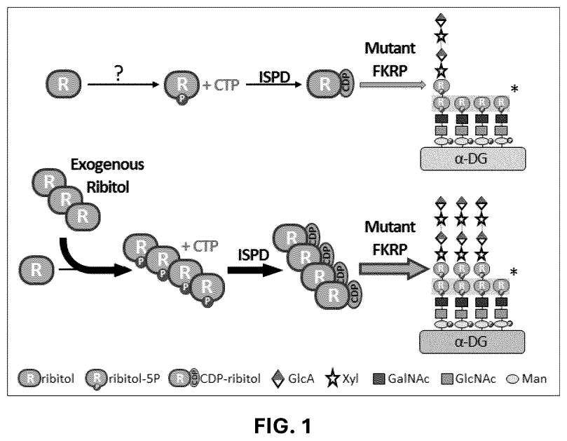

### DETAILED DESCRIPTION

The present disclosure provides methods of monitoring and/or evaluating subjects (e.g., subjects undergoing a treatment such as with ribitol) with a dystroglycanopathy such as Walker-Warburg Syndrome, Muscle Eye Brain Disease, Fukuyama Congenital Muscular Dystrophy (CMD), or a Limb Girdle Muscular Dystrophy (e.g., LGMD2I/R9). The methods provided herein may comprise assessing an amount of alpha-dystroglycan (αDG), or a glycosylated form thereof. In some instances, the methods provided herein may comprise determining a change in an amount of glycosylated αDG for a subject over a period of time. Methods of assessing αDG and glycosylated forms thereof may comprise Western Blot techniques.

The cell membrane protein, dystroglycan, plays a crucial role in connecting the cytoskeleton of a variety of mammalian cells to the extracellular matrix. The α-subunit of dystroglycan (αDG) is characterized by its high level of glycosylation, including a unique O-mannosyl matriglycan. This specific glycosylation enables αDG to bind its matrix ligands effectively. A subset of muscular dystrophies, called dystroglycanopathies, are associated with aberrant, dysfunctional glycosylation of αDG. This defect prevents myocytes from attaching to the basal membrane, leading to contraction-induced injury.

The present disclosure provides a Western blot (WB) assay for determining levels of αDG glycosylation in skeletal muscle tissue. The assay may comprise extracting proteins from fine needle tibialis anterior (TA) biopsies and separation using SDS-PAGE followed by WB. The assay may further comprise detection of glycosylated and core αDG in a multiplexed format using fluorescence antibodies. In some instances, the assay may be useful in monitoring and/or evaluating subjects diagnosed with limb girdle muscular dystrophy type 2i (LGMD2I/R9). As described in Example 5, application of the assay described herein to samples from subjects diagnosed with LGMD2I/R9, which involved quantitative analysis of the WB using a normal TA derived standard curve, revealed significantly reduced levels of αDG in patient biopsies relative to unaffected or normal TA. Moreover, the assay was able to distinguish between the L276I homozygous subgroup and a more severe form of clinical disease observed with other (e.g., heterozygous) FKRP variants. Data demonstrating the accuracy and reliability of the assay described herein are also presented, supporting the potential utility of this assay to monitor changes in αDG of TA muscle biopsies in the evaluation of potential therapeutics.

In an aspect, the present disclosure provides a method comprising a) determining a difference between a first amount of a glycosylated form of alpha-dystroglycan (αDG) in a first sample taken from a subject undergoing treatment for a dystroglycanopathy at a first time and a second amount of αDG in a second sample taken from the subject at a second time; and b) based at least in part on a), determining that the treatment should be continued, ii) determining that the treatment should be discontinued, or iii) determining that the treatment should be adjusted.

In an aspect, the present disclosure provides a method of evaluating a subject (e.g., a human subject) undergoing treatment for a dystroglycanopathy (e.g., LGMD2I/R9), comprising: a) providing a first sample containing a first amount of a glycosylated form of alpha-dystroglycan (αDG) from a subject, wherein the first sample is taken from the subject at a first time; b) providing a second sample containing a second amount of a glycosylated form of αDG from the subject, wherein the second sample is taken from the subject at a second time later than the first time; c) identifying (e.g., determining) the first amount in the first sample; d) identifying (e.g., determining) the second amount in the second sample; and e) determining a difference between the first amount and the second amount.

In some embodiments, c) and d) are performed at the same time. In some embodiments, c) and d) are performed at different times. In some embodiments, the first sample is stored until the second sample is available for analysis. In some embodiments, the first sample is analyzed prior to collection of the second sample.

In some embodiments, the first time is prior to commencement of the treatment for the dystroglycanopathy. A sample taken prior to commencement of the treatment for the dystroglycanopathy may be used to establish a baseline of glycosylation of αDG for the subject. In subjects with dystroglycanopathies, glycosylation of αDG is often reduced compared to that for healthy subjects. Accordingly, a successful treatment regimen may increase the glycosylation of αDG for the subject over time.

In some embodiments, the second time is during or after the treatment of the dystroglycanopathy. In some embodiments, the second time is during the treatment of the dystroglycanopathy. In some embodiments, the second time is at least about 3 months after the first time, such as at least about 6 months, about 9 months, about 12 months, about 15 months, about 18 months, about 21 months, about 2 years, or longer after the first time. In some embodiments, the second time is about 3 months after the first time, such as at least about 6 months, about 9 months, about 12 months, about 15 months, about 18 months, about 21 months, about 2 years, or longer after the first time. In some embodiments, the second time is about 3 months after the first time.

In some embodiments, the first sample and the second sample are biopsy samples (e.g., a tissue biopsy samples). In some embodiments, the first sample and the second sample are derived from muscle tissue. In some embodiments, the first sample and/or the second sample are derived from pectoralis, deltoid, diaphragm, heart, gastrocnemius, quadricep, or tibialis anterior tissue. In some embodiments, the first sample and/or the second sample are derived from cardiac, smooth, or skeletal muscle tissue. In some embodiments, the first sample and/or the second sample are derived from walls of blood vessels, walls of stomach, ureters, intestines, aorta, iris of the eye, prostate, gastrointestinal tract, respiratory tract, small arteries, arterioles, reproductive tracts, veins, glomeruli of the kidneys, bladder, uterus, arrector pili of the skin, ciliary muscle, sphincter, trachea, bile duct, digestive tract, bronchi, or bronchioles. In some embodiments, a sample (e.g., the first sample and the second sample) are tibialis anterior (TA) samples.

In some embodiments, c) and d) comprise determining the first amount and the second amount. In some embodiments, c) and d) comprise performing a Western blotting analysis (e.g., as described herein). In some embodiments, a Western Blotting analytical method comprises performing gel electrophoresis (e.g., SDS-PAGE).

In some embodiments, the Western blotting analysis comprises i) providing a sample comprising a tibialis anterior biopsy, ii) processing the sample with a lysis buffer (e.g., a buffer containing one or more of Tris, glycerol, β-mercaptoethanol, urea, RIPA, SDS, and protease inhibitor) optionally with one or more steel beads to extract proteins; iii) separating proteins on a polyacrylamide gel; iv) incubating proteins on a prepared membrane (e.g., a nitrocellulose membrane); and v) detecting proteins on the membrane (e.g., using fluorescence).

In some embodiments, the lysis buffer comprises SDS and urea. In some embodiments, the lysis buffer comprises RIPA.

In some embodiments, the prepared membrane is incubated with a first primary antibody and a second primary antibody. In some embodiments, the first primary antibody is an anti-alpha-dystroglycan (αDG) antibody clone, such as IIH6C4 (e.g., as described herein). In some embodiments, the second primary antibody is an αDG antibody such as AF6868 (e.g., as described herein). In some embodiments, the membrane incubated with the first and second primary antibodies is incubated with a mixture of secondary antibodies, such as antibodies selective for illumination at 700 nm and 800 nm (e.g., 680RD and Fluor 790, respectively).

In some embodiments, detecting proteins on the membrane comprises illuminating the membrane with one or more wavelengths and collecting one or more images. In some embodiments, detecting proteins on the membrane comprises collecting signal at about 700 nm and 800 nm. In some embodiments, the amount of protein within one or more regions of interest of the membrane are determined. In some embodiments, a first region of interest is 125-260 kDa (e.g., to capture signals of fully glycosylated αDG at 157 kDa). In some embodiments, a first region of interest is 70-260 kDa.

In some embodiments, determining the first amount and the second amount comprises integrating a signal over a range of interest (e.g., as described herein) and interpolating the signal to a standard curve. In some embodiments, the interpolation is performing using regression to a quadratic equation. In some embodiments, the standard curve is based on amounts of glycosylated αDG measured for healthy subjects not undergoing treatment for a dystroglycanopathy.

In some embodiments, determining the first amount and the second amount comprises contacting the sample with one or more antibodies. In some embodiments, the one or more antibodies comprise one or more monoclonal antibodies. In some embodiments, the one or more antibodies comprise one or more polyclonal antibodies. In some embodiments, the one or more antibodies are selected from AF6868 alpha-dystroglycan, IIH6C4 alpha-dystroglycan, IR800CW Mouse Anti-Sheep, and IR680 Goat Anti-Mouse antibodies.

In some embodiments, the amount of αDG in a sample is determined by probing a membrane (e.g., a PVDF or nitrocellulose membrane) with AF6868 Human α-dystroglycan antibody. In some embodiments, the antibody is used at a concentration of about 1 microgram per milliliter (μg/mL). In some embodiments, the antibody was used at a concentration of at least about 0.1 μg/mL, 0.5 μg/mL, 1 μg/mL, 2 μg/mL, 3 μg/mL, 4 μg/mL, 5 μg/mL, 6 μg/mL, 7 μg/mL, or greater. In some embodiments, the antibody is used at a concentration of at most about 7 μg/mL, 6 μg/mL, 5 μg/mL, 4 μg/mL, 3 μg/mL, 2 μg/mL, 1 μg/mL, 0.5 μg/mL, 0.1 μg/mL, or lower.

In some embodiments, the amount of glycosylated αDG in a sample is determined by probing a membrane (e.g., a PVDF or nitrocellulose membrane) with anti α-dystroglycan antibody clone IIH6C4. In some embodiments, the antibody is used at a dilution of about 1:1000. In some embodiments, the antibody was used at a dilution of at least about 1:100, 1:500, 1:1000, 1:2000, 1:3000, 1:4000, 1:5000, 1:7000, 1:10,000, 1:12,000, 1:15,000, 1:20,000, or greater. In some embodiments, the antibody is used at a dilution of at most about 1:20,000, 1:15,000, 1:10,000, 1:7000, 1:5000, 1:4000, 1:3000, 1:2000, 1:1000, 1:500, 1:100, or lower.

In some embodiments, the amount of αDG and/or glycosylated αDG in a sample is determined by contacting the sample with one or more secondary antibodies. In some embodiments, the amount of αDG and/or glycosylated αDG in a sample is determined by probing a membrane (e.g., a PVDF or nitrocellulose membrane) with IR790 Mouse Anti-Sheep (Light chain) antibody. In some embodiments, the antibody is used at a dilution of about 1:5000. In some embodiments, the antibody is used at a dilution of at least about 1:100, 1:500, 1:1000, 1:2000, 1:3000, 1:4000, 1:5000, 1:7000, 1:10,000, 1:12,000, 1:15,000, 1:20,000, or greater. In some embodiments, the antibody is used at a dilution of at most about 1:20,000, 1:15,000, 1:10,000, 1:7000, 1:5000, 1:4000, 1:3000, 1:2000, 1:1000, 1:500, 1:100, or lower.

In some embodiments, the amount of αDG and/or glycosylated αDG in a sample is determined by probing a membrane (e.g., a PVDF or nitrocellulose membrane) with IR680 Goat Anti-Mouse (Light chain) antibody. In some embodiments, the antibody is used at a dilution of about 1:5000. In some embodiments, the antibody is used at a dilution of at least about 1:100, 1:500, 1:1000, 1:2000, 1:3000, 1:4000, 1:5000, 1:7000, 1:10,000, 1:12,000, 1:15,000, 1:20,000, or greater. In some embodiments, the antibody is used at a dilution of at most about 1:20,000, 1:15,000, 1:10,000, 1:7000, 1:5000, 1:4000, 1:3000, 1:2000, 1:1000, 1:500, 1:100, or lower.

In some embodiments, the amount of αDG and/or glycosylated αDG in a sample is determined by visualizing the signals obtained from the imaging of the membrane (e.g., a PVDF or nitrocellulose membrane) that has been contacted with antibodies (e.g., as described herein). In some embodiments, the membrane is visualized on an Odyssey CLx™ imager (LiCor™) and/or a BioRad ChemiDoc MP analyzer and intensity of a first channel (e.g., 700 nanometers (nm) channel) is recorded. In some embodiments, the membrane is visualized on an Odyssey CLx™ imager and/or a BioRad ChemiDoc MP analyzer and intensity of a second channel (e.g., 800 nm channel) is recorded. In some embodiments, the intensity of first and second channels (e.g., 700 and 800 nm channels) are both recorded. In some embodiments, the intensity of first and second channels (e.g., 700 and 800 nm channels) are recorded simultaneously. In some embodiments, determining the first amount and the second amount comprises detection of a fluorescent signal at about 700 nanometers (nm).

In some embodiments, the amount of αDG and glycosylated αDG in a sample is proportional to the signal at the wavelength corresponding to the secondary antibodies used to detect the primary antibodies. In some embodiments, an amount of αDG is determined by the detected intensity in a first channel, which may be an 800 nm emitting channel. In some embodiments, an amount of glycosylated αDG is determined by the detected intensity in a second channel, which may be a 700 nm emitting channel. In some embodiments, a relative amount of glycosylated αDG is determined. In some embodiments, an absolute amount of αDG is determined (e.g., based on comparison to a standard curve, as described herein). In some embodiments, the signal intensities of the 800 and 700 nm channels are used to compute a ratio of glycosylated αDG to total αDG, which ratio can be used to assess the proportion of αDG protein that is glycosylated. Such a ratio can be compared to ratios corresponding to other samples (e.g., as described herein) to track changes in glycosylation over time, in response to changes in therapy and/or dosing, etc.

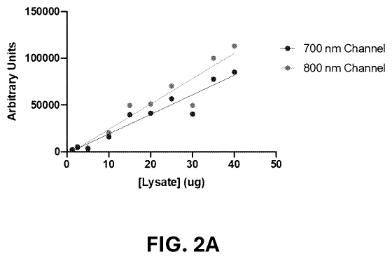

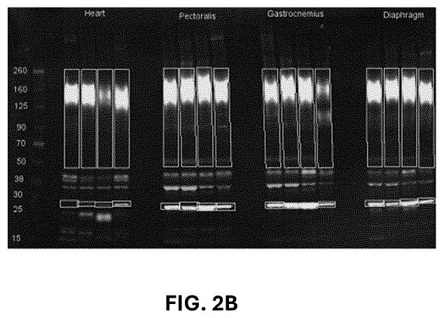

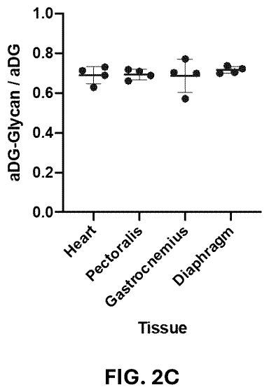

In some embodiments, the intensity of the 700 and 800 nm channels is quantitated by defining an area (e.g., in the form of a rectangle) around a relevant lane in a gel (e.g., as described herein) and measuring the intensity within that area to measure total protein, as depicted in FIG. 2B. In some embodiments, signal intensities from the 700 and 800 channels are normalized to the measurement of total protein. In some embodiments, the ratio of the normalized 700 nm signal to the normalized 800 nm signal is quantitated (e.g., the interpolated value for the 700 nm channel is divided by the interpolated value for the 800 nm channel). In some embodiments, the normalized signal intensity of the 800 nm channel is quantified using the formula:

$$Total α DG / Total protein = Normalized Total α DG$$

In some embodiments, the normalized signal intensity of the 700 nm channel is quantified using the formula: Glycosylated αDG/Total protein=Normalized Glycosylated αDG

In some embodiments, the amount of glycosylated αDG in a sample is determined using the formula: Normalized Glycosylated αDG/Normalized Total αDG=Glycosylated αDG

In some embodiments, an amount of αDG is determined by selecting the 800 nm channel and defining an area (e.g., in the form of a rectangle) around the region from 50-260 kDa in a lane of a probed membrane and measuring the intensity within that area. In some embodiments, an amount of αDG is determined by selecting 800 nm channel and defining an area (e.g., in the form of a rectangle) around the region from 180-260 kDa, 170-260 kDa, 160-260 kDa, 150-260 kDa, 140-260 kDa, 130-260 kDa, 125-260 kDa, 120-260 kDa, 110-260 kDa, 100-260 kDa, 90-260 kDa, 80-260 kDa, 70-260 kDa, 60-260 kDa, 50-260 kDa, 40-260 kDa, 30-260 kDa, 20-260 kDa, or 10-260 kDa in a lane of a probed membrane and measuring the intensity within that area.

In some embodiments, an amount of glycosylated αDG is determined by selecting the 700 nm channel and defining an area (e.g., in the form of a rectangle) around the region from 125-260 kDa in a lane of a probed membrane and measuring the intensity within that area. In some embodiments, an amount of glycosylated αDG is determined by selecting the 700 nm channel and defining an area (e.g., in the form of a rectangle) around the region from 50-260 kDa in a lane of a probed membrane and measuring the intensity within that area. In some embodiments, an amount of αDG is determined by selecting 800 nm channel and defining an area (e.g., in the form of a rectangle) around the region from 180-260 kDa, 170-260 kDa, 160-260 kDa, 150-260 kDa, 140-260 kDa, 130-260 kDa, 125-260 kDa, 120-260 kDa, 110-260 kDa, 100-260 kDa, 90-260 kDa, 80-260 kDa, 70-260 kDa, 60-260 kDa, 50-260 kDa, 40-260 kDa, 30-260 kDa, 20-260 kDa, or 10-260 kDa in a lane of a probed membrane and measuring the intensity within that area.

In some embodiments, an antibody is used to determine an amount of αDG having a molecular weight of between about 50 kDa and about 300 kDa. In some embodiments, an antibody is used to determine an amount of αDG having a molecular weight of between about 50 kDa and about 260 kDa. In some embodiments, an antibody is used to determine an amount of αDG having a molecular weight of between about 100 kDa and about 260 kDa. In some embodiments, an antibody is used to determine an amount of αDG having a molecular weight of between about 125 kDa and about 260 kDa. In some embodiments, an antibody is used to determine an amount of αDG having a molecular weight of between about 150 kDa and about 260 kDa. In some embodiments, an antibody is used to determine an amount of αDG having a molecular weight of between about 200 kDa and about 260 kDa. In some embodiments, an antibody is used to determine an amount of αDG having a molecular weight of between about 260 kDa and about 300 kDa.

In some embodiments, an antibody is used to determine an amount of αDG having a molecular weight of about 50 kDa, about 70 kDa, about 90 kDa, about 1200 kDa, about 150 kDa, about 180 kDa, about 200 kDa, about 230 kDa, about 260 kDa, about 280 kDa, or about 300 kDa.

In some embodiments, an antibody is used to determine an amount of the glycosylated form of αDG having a molecular weight of between about 50 kDa and about 300 kDa. In some embodiments, an antibody is used to determine an amount of αDG having a molecular weight of between about 50 kDa and about 260 kDa. In some embodiments, an antibody is used to determine an amount of αDG having a molecular weight of between about 100 kDa and about 260 kDa. In some embodiments, an antibody is used to determine an amount of αDG having a molecular weight of between about 125 kDa and about 260 kDa. In some embodiments, an antibody is used to determine an amount of αDG having a molecular weight of between about 150 kDa and about 260 kDa. In some embodiments, an antibody is used to determine an amount of αDG having a molecular weight of between about 200 kDa and about 260 kDa. In some embodiments, an antibody is used to determine an amount of αDG having a molecular weight of between about 260 kDa and about 300 kDa.

In some embodiments, an antibody is used to determine an amount of the glycosylated form of αDG having a molecular weight of about 50 kDa, about 70 kDa, about 90 kDa, about 1200 kDa, about 150 kDa, about 180 kDa, about 200 kDa, about 230 kDa, about 260 kDa, about 280 kDa, or about 300 kDa.

In some embodiments, an antibody of the one or more antibodies is used to determine an amount of αDG and/or an amount of the glycosylated form of αDG having a molecular weight of between about 125 kDa and about 260 kDa.

In some embodiments, an antibody of the one or more antibodies is used to determine an amount of the glycosylated form of αDG having a molecular weight in the range of interest of between about 125 kiloDaltons (kDa) and about 260 kDa.

In some embodiments, determining an amount of αDG in a sample comprises contacting the sample with an anti-αDG antibody. In some embodiments, determining an amount of a glycosylated form of αDG in the sample comprises contacting the sample with a matriglycan-specific αDG antibody. In some embodiments, the anti-αDG antibody is AF6868 alpha-dystroglycan antibody. In some embodiments, the matriglycan-specific αDG antibody is IIH6C4 alpha-dystroglycan antibody.

In some embodiments, the anti-αDG antibody detects an N-terminal fragment of αDG having a molecular weight of about 27 kDa.

In some embodiments, the subject has been diagnosed with a dystroglycanopathy. In some embodiments, the subject is undergoing treatment for a dystroglycanopathy. In some embodiments, the dystroglycanopathy is Walker-Warburg Syndrome, Muscle Eye Brain Disease, Fukuyama Congenital Muscular Dystrophy (CMD), or a Limb Girdle Muscular Dystrophy. In some embodiments, the dystroglycanopathy is limb girdle muscular dystrophy type 2i (LGMD2I/R9), LGMD type 2m (LGMD2m), or LGMD type 2u (LGMD2u). In some embodiments, the dystroglycanopathy is limb girdle muscular dystrophy type 2i (LGMD2I/R9). In some embodiments, the dystroglycanopathy is associated with a defect in fukutin (FKTN), Fukutin-related protein (FKRP), isoprenoid synthase domain-containing protein (ISPD), LARGE, or a combination thereof. In some embodiments, the dystroglycanopathy is associated with an FKRP mutation. In some embodiments, the dystroglycanopathy is associated with an FKTN mutation. In some embodiments, the dystroglycanopathy is associated with an ISPD mutation. In some embodiments, the dystroglycanopathy is associated with a LARGE mutation.

In some embodiments, the subject is suspected to have a dystroglycanopathy (e.g., as described herein). In some embodiments, the subject is suspected to have limb girdle muscular dystrophy type 2i (LGMD2I/R9), LGMD type 2m (LGMD2m), or LGMD type 2u (LGMD2u). In some embodiments, the method comprises determining that the subject has a dystroglycanopathy (e.g., as described herein). In some embodiments, determining that the subject has dystroglycanopathy is based at least in part on the amount of the glycosylated form of αDG in a sample from the subject assessed as described herein.

In some embodiments, the method comprises providing a recommendation to administer an additional therapy or therapeutic agent to the subject. In some embodiments, providing a recommendation to administer a therapeutic agent (e.g., ribitol) to the subject is based at least in part on the difference in the amount of the glycosylated form of αDG in a sample from the subject taken at a second time and that in a sample taken at a first time.

In some embodiments, the treatment for the dystroglycanopathy comprises administration of a therapeutic agent. In some embodiments, the therapeutic agent is ribitol or a pharmaceutical composition comprising the same. In some embodiments, the method further comprises f) based at least in part on the difference determined in e), i) determining that the treatment should be continued, ii) determining that the treatment should be discontinued, or iii) determining that the treatment should be adjusted. In some embodiments, iii) comprises determining that a dose of the therapeutic agent should be increased. In some embodiments, iii) comprises determining that a dose of the therapeutic agent should be decreased.

Ribitol, also known as adonitol or (2R,3S,4S)-Pentane-1,2,3,4,5-pentol, has the chemical structure below and molecule weight of 152.15 g/mol.

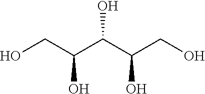

In further embodiments, in place of ribitol, a ribitol derivative may be administered. The ribitol derivative may be a tri-acetylated ribitol; per-acetylated ribitol, ribose; a phosphorylated ribitol (e.g., ribose-5-P); a nucleotide form of ribitol (e.g., a nucleotide-alditol having cytosine or other bases as the nucleobase with 1, 2 or 3 phosphate groups and ribitol as the alditol portion, such as CDP-ribitol or CDP-ribitol-OAc2); or combinations thereof.

In some embodiments, the treatment comprises administering an effective amount of ribitol to the subject. In some embodiments, the treatment comprises administering at least 0.5 grams of ribitol per day (g/day), at least 1 g/day, at least 2 g/day, at least 3 g/day, at least 4 g/day, at least 5 g/day, at least 7.5 g/day, at least 10 g/day, at least 12.5 g/day, at least 15 g/day, at least 20 g/day, at least 25 g/day, at least 30 g/day, at least 35 g/day, at least 40 g/day, at least 45 g/day, at least 50 g/day, at least 55 g/day, or at least 60 g/day to the subject. In some embodiments, the treatment comprises administering 0.5 grams of ribitol per day (g/day), 1 g/day, 1.5 g/day, 2 g/day, 3 g/day, 4 g/day, 5 g/day, 6 g/day, 7.5 g/day, 10 g/day, 12 g/day, 12.5 g/day, 15 g/day, 20 g/day, 25 g/day, 30 g/day, 35 g/day, 40 g/day, 45 g/day, 50 g/day, 55 g/day, or 60 g/day. In some embodiments, the treatment comprises administering ribitol at a dose effective to achieve an area under the concentration-time curve (AUC0-24) steady state level of between 100 (μg·h)/mL and 8000 (μg·h)/m. In some embodiments, ribitol is administered once, twice, three times, or four times daily.

In some embodiments, the method further comprises evaluating the subject using the North Star Assessment for Limb Girdle Type Muscular Dystrophies (NSAD), Performance of upper limb test 2.0 (PUL2.0), 10-meter walk test (10MWT), 100-meter timed test (100MTT), forced vital capacity (FVC) assessment, serum creatine kinase (CK) levels, or a combination thereof.

In some embodiments, the second amount is greater than the first amount. In some embodiments, the second amount is at least about 10%, about 20%, about 30%, about 40%, about 50%, about 60%, about 70%, about 80%, about 90%, about 100%, about 120%, about 140%, about 160%, about 180%, about 200%, about 250%, about 300%, about 350%, about 400%, about 450%, about 500%, or greater than the first amount. In some embodiments, the second amount is at least double the first amount.

In some embodiments, the method further comprises determining a first amount of total αDG in the first sample and a second amount of total αDG in the second sample. In some embodiments, the method further comprises determining (i) a first ratio of the glycosylated form of αDG to the total αDG in the first sample and (ii) a second ratio of the glycosylated form of αDG to the total αDG in the second sample.

In some embodiments, greater than 2 samples from the subject are evaluated as described herein. In some embodiments, at least 3, at least 4, at least 5, at least 6, at least 7, at least 8, at least 9, at least 10, at least 15, at least 25, at least 50, at least, 75 or at least 100 samples are collected from the subject. In some embodiments, the timepoint of each successive sample collection is at a timepoint which is later than the previous sample collection (e.g., a first sample collected at a first timepoint and a second sample collected at a second timepoint, wherein the second time point is later than the first timepoint.)

For example, the first sample is collected from the subject at a first timepoint, a second sample is collected from the subject at a second timepoint, a third sample is collected from the subject at a third timepoint, a fourth sample is collected from the subject at a fourth timepoint, and a fifth sample is collected from the subject at a fifth timepoint. In an embodiment, the second timepoint is later than the first timepoint, the third timepoint is later than the first and second timepoints, the fourth timepoint is later than the first, second, and third timepoints, the fifth timepoint is later than the first, second, third, and fourth timepoints.

In some embodiments, the method comprises determining a relative amount of alpha-dystroglycan (αDG) in the first and/or second sample, determining a relative amount of the glycosylated form of αDG in the first and/or second sample, and determining the first and/or second ratio of the relative amount of the glycosylated form of αDG to the relative amount of αDG in the first and/or second sample. In some embodiments, determining a relative amount of alpha-dystroglycan (αDG) in the first and/or second sample and determining a relative amount of the glycosylated form of αDG in the first and/or second sample comprises performing a Western Blotting analytical method (e.g., as described herein).

The present disclosure also provides a method of assessing the glycosylation of αDG in cells and tissue samples from subjects with dystroglycanopathies to assess longitudinal disease progression. Furthermore, the present disclosure provides a method of evaluating the impact of therapeutics that seek to restore the glycosylation of αDG and may assist in guiding dose selection.

The present disclosure also provides a method of assessing the effect of therapeutic agents that increases the glycosylated form of αDG by assessing biopsy samples before and after treatment with a therapeutic agent. A change in the glycosylated form of αDG and/or the restoration of αDG with treatment could be used to guide dose selection for patients with dystroglycanopathies.

In some embodiments, αDG expression in a subject with at least one mutation in the FKRP gene is increased following ribitol treatment as compared to an untreated subject. In some embodiments, the ratio of glycosylated αDG to total αDG in a subject with at least one mutation in the FKRP gene is increased following ribitol treatment as compared to an untreated subject. In some embodiments, the ratio of glycosylated αDG to total αDG in a subject with at least one mutation in the FKRP gene detects an increase in αDG glycosylation following ribitol treatment as compared to an untreated subject.

The present disclosure provides a method of assessing the glycosylation of αDG in cells and tissue samples from subjects having or suspected of having a disorder associated with (e.g., caused by or resulting from) a mutation in a fukutin related protein (FKRP) gene. The method comprises determining an amount of a glycosylated form of αDG in a sample from a subject having or suspected of having a disorder associated with a mutation in a fukutin related protein (FKRP) (e.g., as described herein). In some embodiments, the method further comprises comparing the amount of the glycosylated form of αDG in the sample to an amount of the glycosylated form of αDG in a sample from one or more healthy subjects (e.g., using a standard curve, as described herein) and, based at least in part on the difference between these amounts, i) diagnosing the subject with the disorder, and/or ii) providing a recommendation for a particular treatment regimen (e.g., treatment with a therapeutic agent such as ribitol, as described herein) for the subject.

In some embodiments, the subject has one or more mutations in the FKRP gene, such as one or more mutations associated with a dystroglycanopathy such as LGMD2I/R9. In some embodiments, the subject has a single mutation associated with a dystroglycanopathy such as LGMD2I/R9. In some embodiments, the subject harbors a P448L mutation in the FKRP gene. In some embodiments, the subject harbors a L276I mutation in the FKRP gene. In some embodiments, the subject harbors a P448L mutation and/or a L276I mutation in the FKRP gene.

In some embodiments, the present disclosure provides methods of assessing the glycosylation of αDG in cells and tissue samples from subjects to identify subjects for ribitol treatment.

In some embodiments, the present disclosure provides methods of assessing the glycosylation of αDG in cells and tissue samples from subjects to monitor subject's responses to ribitol treatment.

In some embodiments, the present disclosure provides methods of assessing the glycosylation of αDG in cells and tissue samples from subjects to assess and/or amend dosing of subjects with ribitol treatment.

Additionally, the present disclosure provides a method of assessing the glycosylation of αDG in cells and tissue samples from subjects with muscle weakness wherein the subject is a carrier of a mutated FKRP gene and/or with a mutation in a dystroglycan-related gene and/or with a defect in glycosylation of αDG. The muscle weakness can include but is not limited to weakness of skeletal muscle, cardiac muscle and/or respiratory muscle, in any combination, in the subject.

In some embodiments, the disorder associated with muscle weakness can be associated with a defect in glycosylation of αDG, including situations without clear understanding of the underlying causes for the defect.

In some embodiments, a defect in glycosylation of αDG can be associated with a mutated FKRP gene. In some embodiments, a mutated FKRP gene in a subject results in at least about 5%, 10%, 25%, 35%, 40%, 50%, 55%, 60%, 65%, 70%, 75%, 80%, 85%, 90%, 95%, 96%, 97%, 98%, 99%, or greater reduction in glycosylation of αDG (e.g., relative to a healthy subject not having a dystroglycanopathy such as LGMD2I/R9).

In some embodiments, a defect in αDG expression can be associated with a mutated FKRP gene. In some embodiments, a mutated FKRP gene in a subject results in at least about 5%, 10%, 25%, 35%, 40%, 50%, 55%, 60%, 65%, 70%, 75%, 80%, 85%, 90%, 95%, 96%, 97%, 98%, 99%, or greater reduction in expression of αDG (e.g., relative to a healthy subject not having a dystroglycanopathy such as LGMD2i).

In some embodiments, a defect in the ratio of glycosylation of αDG to total αDG can be associated with a mutated FKRP gene. In some embodiments, a mutated FKRP gene in a subject results in at least about 5%, 10%, 25%, 35%, 40%, 50%, 60%, or 75% reduction in the ratio of glycosylation of αDG to total αDG.

In some embodiments, the amount of glycosylation of αDG or the ratio of glycosylation of αDG to total αDG in a subject provides a biomarker for a recommendation to administer a therapeutic agent to a subject.

In some embodiments, the present disclosure provides a method of assessing the glycosylation of αDG in cells and tissue samples from subjects with muscular dystrophy diseases for which restoration of and/or enhanced glycosylation of αDG would be beneficial and/or therapeutic. A nonlimiting example of a disorder associated with a mutation or loss of function in the FKRP gene is Limb-Girdle Muscular Dystrophy type 2i (LGMD2I/R9). Certain mutations in FKRP are associated with Walker-Warburg Syndrome (WWS) and in congenital muscular dystrophy type 1C (MDC1C). The methods of the present disclosure may also be applied in any disease or disorder associated with metabolism of ribitol and/or any disease or disorder for which ribitol is therapeutically effective.

The methods of this disclosure can be applied to non-muscular dystrophy diseases for which restoration of and/or enhanced glycosylation of αDG would be beneficial and/or therapeutic.

In additional embodiments, the present disclosure provides a method of assessing the glycosylation of αDG in cells and tissue samples from subjects with muscle weakness, e.g., muscle weakness which limits or slows daily activity of the subject. Muscle weakness can imply that a subject is not able to perform the daily activities that a normal person of similar gender, age and other conditions would be expected to be capable of performing. An example is the loss of or lack of ability to climb stairs, run or hold an object for an extended period.

The present disclosure further provides a method of assessing the glycosylation of αDG in cells and tissue samples from subjects with a disorder associated with a defect in glycosylation of αDG. A subject can be suspected of having a defect in glycosylation of αDG if the subject has muscle weakness even in cases where genetic and biochemical analyses of the subject have failed to identify a causative gene defect.

Further provided herein is a method of assessing the glycosylation of αDG in cells and tissue samples from subjects with a disorder associated with a defect in glycosylation of αDG caused by a mutation in the FKRP gene. A mutation in an FKRP gene can be identified by genetic analysis of the nucleic acid of a subject.

In some embodiments, a therapeutic agent for use in the compositions and methods described herein can be ribitol.

In further embodiments, the methods of the disclosure comprise, in place of ribitol, administering a ribitol derivative. The ribitol derivative may be a tri-acetylated ribitol; per-acetylated ribitol, ribose; a phosphorylated ribitol (e.g., ribose-5-P); a nucleotide form of ribitol (e.g., a nucleotide-alditol having cytosine or other bases as the nucleobase with 1, 2 or 3 phosphate groups and ribitol as the alditol portion, such as CDP-ribitol or CDP-ribitol-OAc2); or combinations thereof.

In some embodiments, the disease or disorder is associated with a defect in Fukutin-related protein (FKRP). In some embodiments, the disease or disorder is associated with a defect in fukutin (FKTN). In additional embodiments, a subject has a mutation in a gene encoding fukutin (FKTN), fukutin-related protein (FKRP), isoprenoid synthase domain-containing protein (ISPD), or LARGE, or a combination thereof, that causes a partial or complete loss-of-function in FKRP.

In some embodiments, the therapeutic agent of the present disclosure may improve and/or prevent one or more symptoms of disease, including but not limited to limb muscle weakness, e.g., with mild calf and thigh hypertrophy; decreased hip flexion and adduction; decreased knee flexion and ankle dorsiflexion; progressive muscle degeneration; fiber wasting; decreased matriglycan expression; infiltration and accumulation of fibrosis and/or fat in muscle tissues; loss of ambulation. Muscle weakness may involve the diaphragm with varying severity, leading to respiratory failure in a proportion of patients. Cardiac muscle is affected with the most severe and frequent presentation being dilated cardiomyopathy.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description is for the purpose of describing particular embodiments only and is not intended to be limiting. All publications, patent applications, patents and other references mentioned herein are incorporated by reference in their entirety. In case of a conflict in terminology, the present specification is controlling.

As used in the description of the invention and the appended claims, the singular forms “a”, “an”, and “the” are intended to include the plural forms as well, unless the context clearly indicates otherwise.

The term “about,” as used herein when referring to a measurable value such as an amount of dose (e.g., an amount of a fatty acid) and the like, is meant to encompass variations of ±20%, ±10%, ±5%, ±1%, ±0.5%, or even ±0.1% of the specified amount.

“Subject” as used herein includes a mammal, such as primate, mouse, rat, dog, cat, cow, horse, goat, camel, sheep or a pig, preferably a human.

“Treat,” “treating,” or “treatment” as used herein also refers to any type of action or administration that imparts a benefit to a subject that has a disease or disorder, including improvement in the condition of the patient (e.g., reduction or amelioration of one or more symptoms), healing, etc.

The term “effective amount” refers to an amount of an agent (e.g., ribitol) sufficient to have desired biochemical or physiological effect. The term “therapeutically effective amount” refers to an amount of an agent (e.g., ribitol) that is sufficient to improve the condition, disease, or disorder being treated and/or achieved the desired benefit or goal (e.g., control of body weight). Those skilled in the art will appreciate that the therapeutic effects need not be complete or curative, as long as some benefit is provided to the subject. The term “amount” when in reference to αDG or glycosylated αDG refers to the quantification of protein as quantified by detection of a signal obtained from the wavelength corresponding to a secondary antibody used to detect a primary antibody.

The term “enhancement,” “enhance,” “enhances,” or “enhancing” refers to an increase in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or an increase in the specified activity of at least about 5%, 10%, 25%, 35%, 40%, 50%, 60%, 75%, 80%, 90%, 95%, 97%, 98%, 99% or 100%.

The term “inhibit,” “diminish,” “reduce” or “suppress” refers to a decrease in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or a decrease or reduction in the specified activity of at least about 5%, 10%, 25%, 35%, 40%, 50%, 60%, 75%, 80%, 90%, 95%, 97%, 98%, 99% or 100%. These terms are intended to be relative to a reference or control.

The above terms are relative to a reference or control. For example, in a method of detecting glycosylation of αDG in a subject sample, the enhancement is relative to the amount of glycosylation of αDG in a subject sample (e.g., a control subject sample) in the absence of administration of ribitol, CDP-ribitol, ribose and/or ribulose. In another example, in a method of detecting glycosylation of αDG in a sample comprising mutant FKRP, the enhancement is relative to the amount of glycosylation of αDG in a sample (e.g., a reference sample) comprising wild type FKRP.

Also as used herein, “and/or” refers to and encompasses any and all possible combinations of one or more of the associated listed items, as well as the lack of combinations when interpreted in the alternative (or).

Unless the context indicates otherwise, it is specifically intended that the various features described herein can be used in any combination. Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed.

It will also be understood that, as used herein, the terms example, exemplary, illustrative, and grammatical variations thereof are intended to refer to non-limiting examples and/or variant embodiments discussed herein and are not intended to indicate preference for one or more embodiments discussed herein compared to one or more other embodiments.

All publications, patent applications, patents and other references cited herein are incorporated by reference in their entireties for the teachings relevant to the sentence and/or paragraph in which the reference is presented.

Unless the context indicates otherwise, it is specifically intended that the various features described herein can be used in any combination.

Moreover, the present disclosure also contemplates that in some embodiments, any feature or combination of features set forth herein can be excluded or omitted.

### EXAMPLES

The following examples are put forth so as to provide those of ordinary skill in the art with a description of how the compositions and methods described herein may be used, made, and evaluated, and are intended to be purely illustrative of the invention and are not intended to limit the scope of what is regarding as the invention.

### Example 1: Method for the Ratio-Metric Detection of Alpha-Dystroglycan Glycosylation

The following is an illustrative, non-limiting method for the assessment of αDG protein and the matriglycan of αDG in muscle homogenates. A multiplexed Western Blot-based method that reports on the relative O-glycosylation of αDG from muscle biopsies is disclosed herein. The results of 13 LGMD2I/R9 patients representing homozygous and compound heterozygous mutations in FKRP are reported. In all patients there was decreased matriglycan expression as compared to healthy human donor tissue. Four of these patients had longitudinal biopsies and there was little change in matriglycan expression from the initial baseline biopsy measurement but significantly reduced matriglycan expression as compared to donor tissue. The quantitative nature of this method may be of use in understanding the longitudinal changes in patients with dystroglycanopathies such as LGMD2I/R9 and in assessing the impact of therapeutics directed at increasing the O-glycosylation of αDG.

Muscle Biopsy: Muscle biopsy samples were collected from tibialis anterior using a fine needle aspirate. In total three passes were collected. Biopsy samples were snap frozen immediately after collection and stored at −80° C. until analysis.

Healthy Human Tissue: Control healthy human muscle tissue (male and female) was obtained and stored at −80° C. until analysis.

Murine Mouse Tissue: Murine muscle tissue samples from healthy, P448L, and L276I FRKP mutant mice were stored at −80° C. until analysis.

Tissue Processing: Tissue samples were processed using a TissueLyzer II™ (Qiagen®). Approximately 250 milligrams (mg) of tissue was placed in a 2 mL microtube with a steel bead and 1 mL of Radioimmunoprecipitation assay buffer (RIPA) buffer supplemented with protease and phosphatase inhibitors. The microtubes were subjected to three cycles of disruption (2 minutes at 25 Hertz (Hz)) in the TissueLyzer II™. Lysates were clarified by centrifugation and the supernatant was transferred to fresh tubes and protein concentration in lysate was determined using a BCA assay (Pierce®).

SDS-PAGE: Polyacrylamide gel electrophoresis was carried out on 4-20% StainFree™ gels. Tissue lysates as described above were diluted to 20 micrograms (μg)/15 microliters (μL) with RIPA buffer in LiCor™ loading buffer. A total of 20 μg of lysate was loaded per well. Pre-stained protein markers were also loaded onto gel. After electrophoretic separation the gel was washed in water and total protein was imaged using the ChemiDoc™ imaging system (BioRad®).

Western Blotting: Proteins were transferred to a PVDF membrane using an iBlot™ Gel Transfer Device (Invitrogen®). The membrane was dried and activated with a methanol wash and then rinsed with water. The membrane was blocked with Intercept blocking buffer (LiCor™) and probed with AF6868 Human α-dystroglycan antibody: 1 μg/mL [anti-αDG antibody] (R&D Systems®) and anti α-dystroglycan antibody clone IIH6C4: 1:1000 [anti-glycosylated-αDG antibody](Millipore®). The primary antibody solution was removed and the washed four times with TBST. The blot was then probed with IR790 Mouse Anti-Sheep (Light chain): 1:5,000 and IR680 Goat Anti-Mouse (Light chain): 1:5,000 secondary antibodies (LiCor™). The secondary antibody solution was removes and the membrane was washed four times with TBST. The membrane was visualized on an Odyssey CLx™ imager (LiCor™) and intensities of the 700 and 800 nm channels were recorded.

Data analysis: Analysis was carried on GraphPad Prism™ (version 8). Signal intensities were normalized to total protein and the ratio of 700 nm normalized signal/800 nm normalized signal was determined. Linear fits were assessed where applicable using the linear regression function of Prism™. Statistical significance was determined using an unpaired t-test using Prism™ (e.g., using the p-value for the parameters slope and intercept).

StainFree gel image is quantitated using ImageLab™ software to analyze the image from the ChemiDoc™ Imaging System. A rectangle is drawn around each lane to measure total protein. Signal intensities were normalized to total protein and the ratio of 700 nm normalized signal/800 nm normalized signal was determined. Linear fits were assessed were applicable using a linear regression function. Statistical significance was determined using an unpaired t-test.

α-Dystroglycan is quantitated using ImageStudio™ Software (LiCor™) to analyze the images from the Odyssey: total α-Dystroglycan is analyzed by selecting 800 channel and drawing a rectangle around the region from 50-260 kDa in each lane, while glycosylated α-Dystroglycan is analyzed by selecting the 700 channel and drawing a rectangle around the region from 50 kDa-260 kDa.

Calculations are performed as follows: 1) Normalize Total α-Dystroglycan: Total α-Dystroglycan/Total protein; 2) Normalize Glycosylated α-Dystroglycan: Glycosylated α-Dystroglycan/Total protein; and 3) Glycosylated α-Dystroglycan: Normalized Glycosylated α-Dystroglycan/Normalized Total α-Dystroglycan.

### Results

To assess the glycosylation of αDG in cells and tissue samples from patients, a multiplexed Western Blot method was used to simultaneously detect αDG and the glycosylated form of αDG.

Using samples obtained from healthy human donor tissue, the signal intensity (800 nm) observed for total α-dystroglycan in heart, pectoralis, gastrocnemius, and diaphragm tissues displayed a linear regression with respect to μg/lane of total lysate (linear range was observed for ˜1.25-25 μg/lane) (data not shown). The signal intensity (700 nm) observed for glycosylated α-dystroglycan in heart, pectoralis, gastrocnemius, and diaphragm tissues also displayed a linear regression with respect to μg/lane of total lysate (linear range was observed for ˜1.25-25 μg/lane) (data not shown).

Muscle tissue from 4 human donors (2 males and 2 females) was assessed for αDG variability.

TABLE 1
Details of donors assessed for αDG variability

| Subject | Details |
|---|---|
| Donor 1 | 41 -year-old African-American male |
| | COD: Cardiopulmonary Arrest following respiratory arrest |
| | Medical history: Type 2 diabetes, hypertension, |
| | myocardial infarction |
| | BMi: 40.7 |
| | Medications: Albuterol, Insulin, HTN medication |
| Donor 2 | 77-year-old Caucasian male |
| | COD: Cardiac - unspecified |
| | Medical History: hypertension, type 2 diabetes, |
| | hyperlipidemia, glaucoma |
| | BMI: 22.1 |
| | Medications: Hypertension and diabetes medications |
| Donor 3 | 75-year-old Caucasian female |
| | COD: Traumatic subdural hematoma following stroke |
| | Medical History: hypertension, adenoma, arthritis, |
| | gastrointestinal reflux disease |
| | BMI: 34.1 |
| | Medications: blood pressure and thyroid medications |
| Donor 4 | 61 -year-old Caucasian female |
| | COD: Respiratory failure |
| | Medical History: cardiovascular accident, atrial fibrillation |
| | BMI: 27.2 |
| | Medications: Anticoagulant and heart rate regulating medications |

As shown in FIG. 2A, gastrocnemius tissue lysate was serially diluted, and the linearity of each detection channel was plotted as a function of lysate concentration. Four different muscle types (heart, pectoralis, gastrocnemius, and diaphragm) were analyzed to assess the variability of the αDG glycan ratio. As shown in FIG. 2B, the Western Blot membrane displays the merge of both detection channels with regions of interest for αDG (˜50-260 kDa) and a putative N-terminal fragment (˜27 kDa) indicated with yellow boxes. As shown in FIG. 2C, the ratio of glycosylated αDG to total αDG protein was determined using intensities from the detection channels and plotted for each muscle type. The extent of glycosylation of αDG was observed to be consistent among each tissue type.

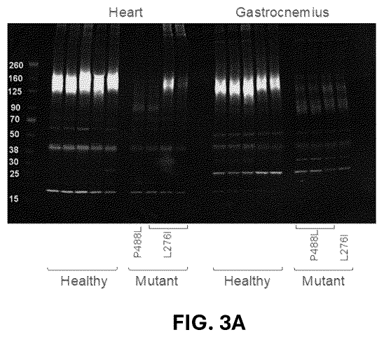

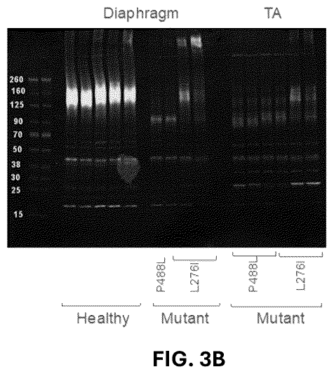

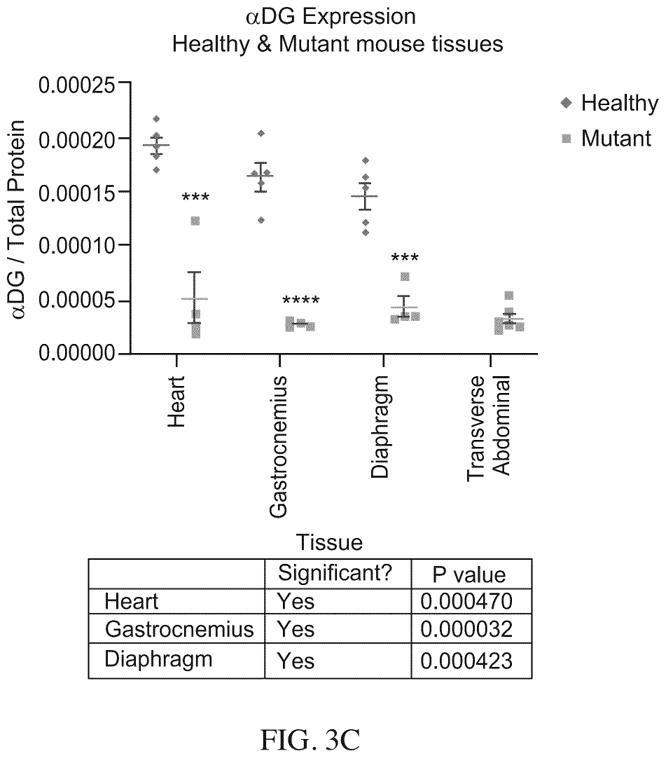

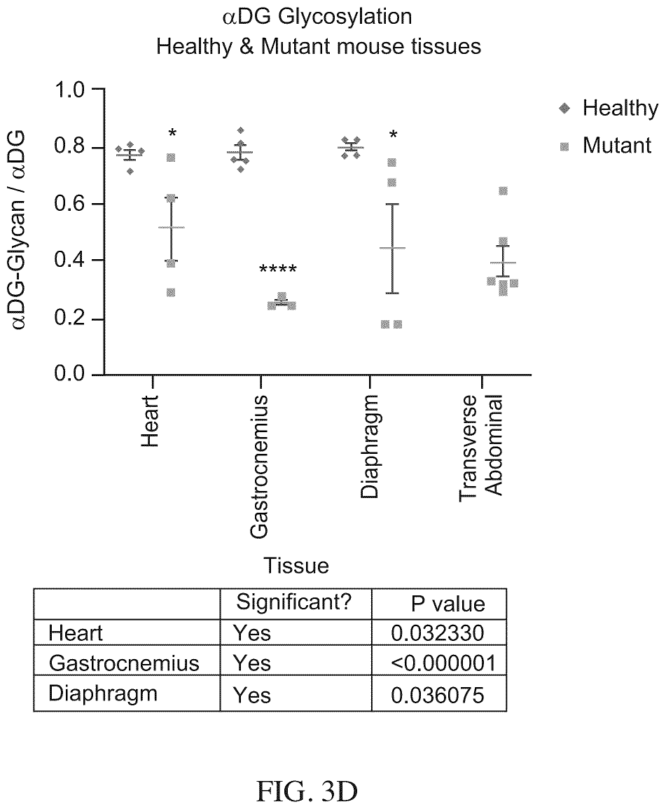

The mutations P448L and L276I in the FKRP protein lead to LGMD2I and result in decreased αDG glycosylation and loss of muscle performance. Samples from rodent models of LGMD2I were analyzed to evaluate decreases in the glycosylation of αDG in muscle tissue. As shown in FIG. 3A, the Western Blot membrane displays total αDG and glycosylated αDG in healthy and mutant mouse heart and gastrocnemius tissues. In FIG. 3B, the Western Blot membrane displays total αDG and glycosylated αDG in healthy and mutant mouse diaphragm and tibialis anterior (TA) tissues. As shown in FIG. 3C, the ratio of total αDG to total protein was determined using intensities in the ˜50-260 kDa range and plotted for each muscle type. In FIG. 3D, the ratio of glycosylated αDG to total αDG was determined using intensities in the ˜50-260 kDa range and plotted for each muscle type. Both αDG expression and glycosylation of αDG was observed to be significantly decreased in all tissue types from mutant animals. This data demonstrated that decreases in αDG were present in mice harboring mutations in FKRP that are known to cause LGMD2I in humans.

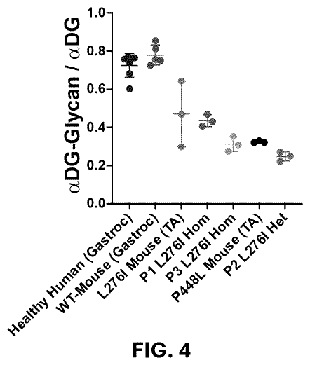

As shown in FIG. 4, the ratio of glycosylated αDG to total αDG was assessed in gastrocnemius muscle tissue from a healthy human donor (Healthy Human) and a wild type mouse (WT-Mouse), TA muscle tissue from a L276I FKRP mutant mouse model (L276I Mouse) and a P448L mutant mouse model (P448L Mouse), and three LGMD2I patients containing a L276I FRKP mutation (two homozygous: [P1 L276I and P3 L276I] and one compound heterozygous: [P2 L276I]). The data show an αDG glycosylation defect in all samples from subjects with FKRP mutations as compared to healthy and wild type subjects. The αDG glycosylation defect was exhibited though statistically significant lower ratios of glycosylated αDG to total αDG in all samples from subjects with FKRP mutations as compared to healthy and wild type subjects. Table 2 summarizes the mean and standard deviation of the measurements and statistical significance.

TABLE 2
Details of samples analyzed according to Example 1

| | | | P Unpaired |
|---|---|---|---|
| Sample | Mean Ratio | SD | t-test |
| Healthy Human (Gastroc) | 0.72 | 0.06 | |
| WT-Mouse (Gastroc) | 0.78 | 0.03 | |
| L276I Mouse (TA) | 0.47 | 0.17 | 0.10 |
| P1 L276I Hom | 0.43 | 0.03 | <0.0001 |
| P3 L276I Hom | 0.31 | 0.04 | <0.0001 |
| P448L Mouse (TA) | 0.32 | 0.005 | <0.0001 |
| P2 L276I Het | 0.25 | 0.02 | <0.0001 |

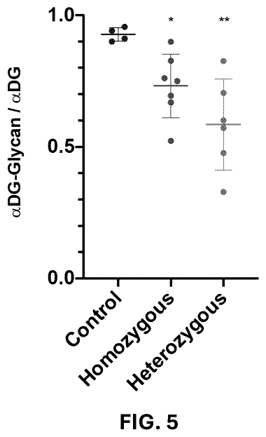

Samples from human LGMD2I patients with mutation L276I were analyzed for αDG glycosylation ratio (FIG. 5).

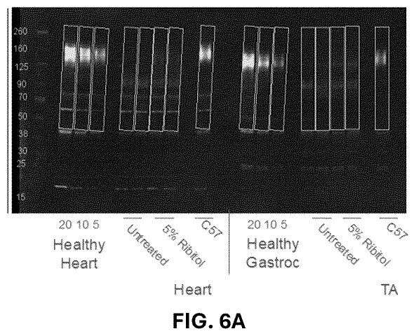

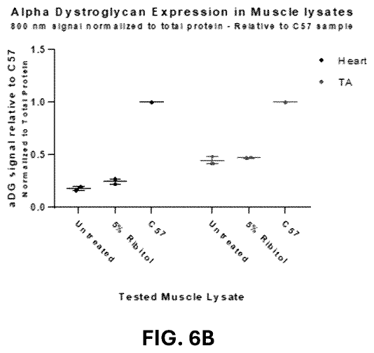

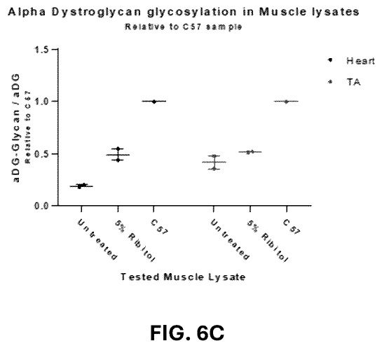

Samples from P448L mutant FKRP mice were analyzed to evaluate glycosylation of αDG following restorative therapy. As shown in FIG. 6A, the Western Blot membrane displays the merge of both detection channels with regions of interest for αDG (˜50-250 kDa) indicated with yellow boxes. The samples included healthy heart muscle tissue, healthy gastrocnemius muscle tissue, and P448L mutant FKRP heart and gastrocnemius tissues that were untreated or treated with 5% Ribitol, and an untreated C57 control for normalization.

As shown in FIG. 6B, total αDG signal in untreated, 5% Ribitol treated, and C57 control samples was quantified using the regions of interest for αDG (˜50-250 kDa) indicated with yellow boxes in FIG. 6A. In FIG. 6C, the ratio of glycosylated αDG to total αDG signal in untreated, 5% Ribitol treated, and C57 control samples was quantified using the regions of interest for αDG indicated in FIG. 6A. The data show that αDG expression is increased in 5% Ribitol treated P488L mice as compared to untreated P488L mice in both heart muscle and TA lysates (FIG. 6B). The data also show that the ratio of glycosylated αDG to total αDG is increased in 5% Ribitol treated P488L mice as compared to untreated P488L mice in both heart muscle and TA lysates (FIG. 6C). The data demonstrate that this assay is capable of detecting increases in αDG glycosylation with a restorative therapy.

### Conclusions

A multiplexed Western Blot method to detect the radiometric extent of αDG glycosylation relative to total αDG from muscle lysates that can be applied to human biopsy samples was demonstrated. The detection signals show linearity with lysate concentration.

Analysis of several muscle types from human donors demonstrated that the αDG ratio is similar across different muscle types. Tibialis anterior from two murine models of LGMD2I/R9 were analyzed using method of the present disclosure and a decrease in the glycation of αDG was observed in both FKRP mutant mouse models with the largest reduction observed in the P448L FKRP mutation. Three biopsies from LGMD2I/R9 patients harboring an L276I mutation displayed statistically significant reductions in αDG glycosylation. Samples from several LGMD2I/R9 patients were analyzed and statistically significant reduction in αDG glycation was observed. Compound heterozygous patient showed a great loss in αDG glycosylation.

The methods of the present disclosure may be used understanding the longitudinal changes in patients with LGMD2I/R9 and in assessing the impact of therapeutics directed at increasing the O-glycosylation of αDG.

### Example 2: Detection of Alpha-Dystroglycan in Muscle Biopsies Using a Multiplexed Western Blot Method

A multiplexed Western Blot approach for detecting αDG in patient biopsies at baseline and post-treatment with, e.g., ribitol is described.

Muscle biopsy: Muscle biopsy samples were collected from tibialis anterior (TA) using a fine needle aspirate. In total three passes were collected. Biopsy samples were snap-frozen immediately after collection and stored at −80° C. until analysis.

Healthy human tissue: Control healthy human muscle tissue (male and female TA) was obtained from BioIVT and stored at −80° C. until analysis.

Tissue lysis: Tissue samples were processed using a TissueLyzer II (Qiagen). Approximately 2-40 mg of tissue was placed in a 2 mL microtube with a steel beed and 1/10 weight/volume (1/v) of SDS-urea lysis buffer supplemented with protease inhibitors. The microtubes were subjected to five cycles of disruption (2 minutes at 25 Hz) in the TissueLyzer II. Lysates were clarified by centrifugation and the supernatant was transferred to fresh tubes and lysate concentration was determined using 660 nm Protein Assay Kit (Pierce).

SDS-PAGE: Polyacrylamide gel electrophoresis was carried out on 4-20% Novex gels (LifeTech). Tissue lysates were diluted to appropriate concentrations in SDS-Urea buffer supplemented with bromophenol blue. Pre-stained protein molecular weight markers were also loaded onto gel.

Western blotting: Proteins were transferred to a nitrocellulose membrane (NC) using an iBlot2 Gel Transfer Device (Invitrogen). The membrane was dried then rinsed with water. The membrane was blocked with Intercept blocking buffer (LiCor) and probed with AF6868 alpha dystroglycan antibody (R&D Systems) and IIH6C4 alpha dysroglycan antibody (Millipore) overnight at 4° C. The primary antibody solution was discarded and then washed four times with TBST. The blot was then probed with IR800CW Mouse Anti-Sheep and IR680 Goat Anti-Mouse secondary antibodies (LiCor) for one hour at room temperature. The secondary antibody solution was discarded, and the membrane was washed four times with TBST. The membrane was visualized on an Odyssey CLx imager (LiCor) and intensities of the 700 and 800 nm channels were recorded.

Data analysis: Analysis was carried out on GraphPad Prism (version 9). Signal intensities were normalized to total protein and the ratio of 700 nm normalized signal/800 nm normalized signal was determined. Linear fits were assessed using the linear regression function of Prism. Statistical significance was determined using an unpaired t-test using Prism.

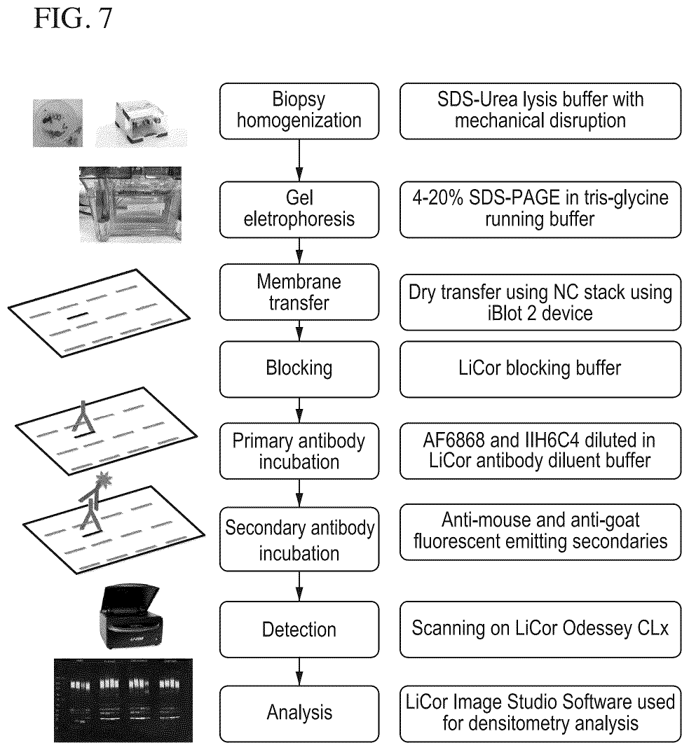

FIG. 7 schematically illustrates the workflow for the Western Blot assay.

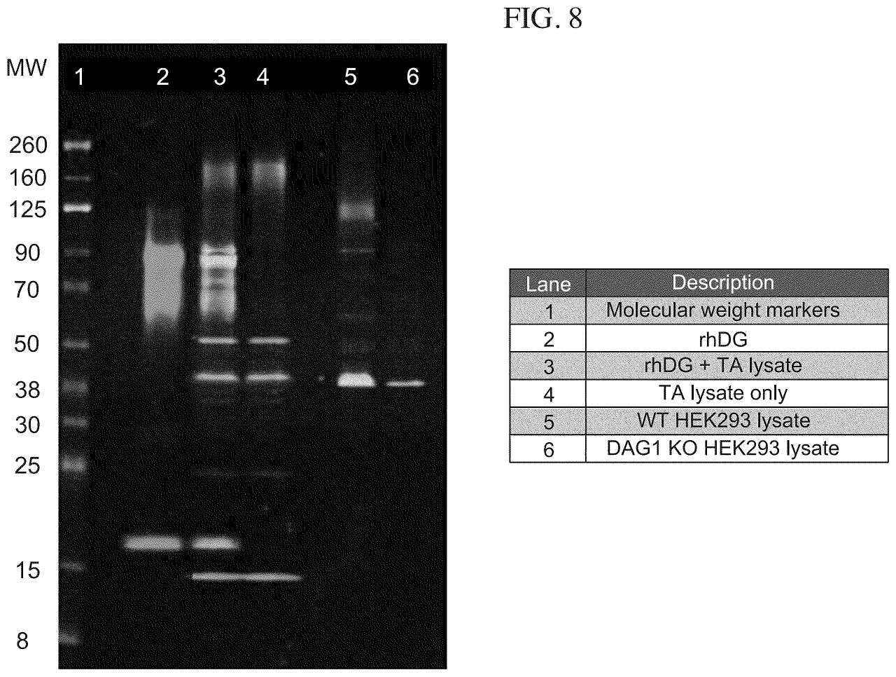

FIG. 8 shows a Western blot merged image showing the specificity of both primary antibodies, as well as the staining of rhDG. Lane 1 shows molecular weight markers; lane 2 shows rhDG; lane 3 shows rhDG+TA lysate; lane 4 shows TA lysate only; lane 5 shows wild type HEK293 lysate; and lane 6 shows DAG1 knockout HEK293 lysate. The specificity of the primary antibodies was assessed using recombinant protein and lysates. Lane 2 is a recombinant human αDG (rhDG) only showing staining for AF6868. Lanes 3 and 4 are healthy donor tibialis anterior (TA) lysates spiked with recombinant human αDG and only TA lysate showing staining with both the AF6868 and IIH6C4 antibodies. Lanes 5 and 6 are lysates from HEK293 and an engineered DAG1 knockout HEK293 cells. The lack of signal in both channels in the 125 kDa for lane 6 demonstrates both antibodies react with αDG as the knockout lacks αDG while the parental HEK293 shows staining by both primary antibodies.

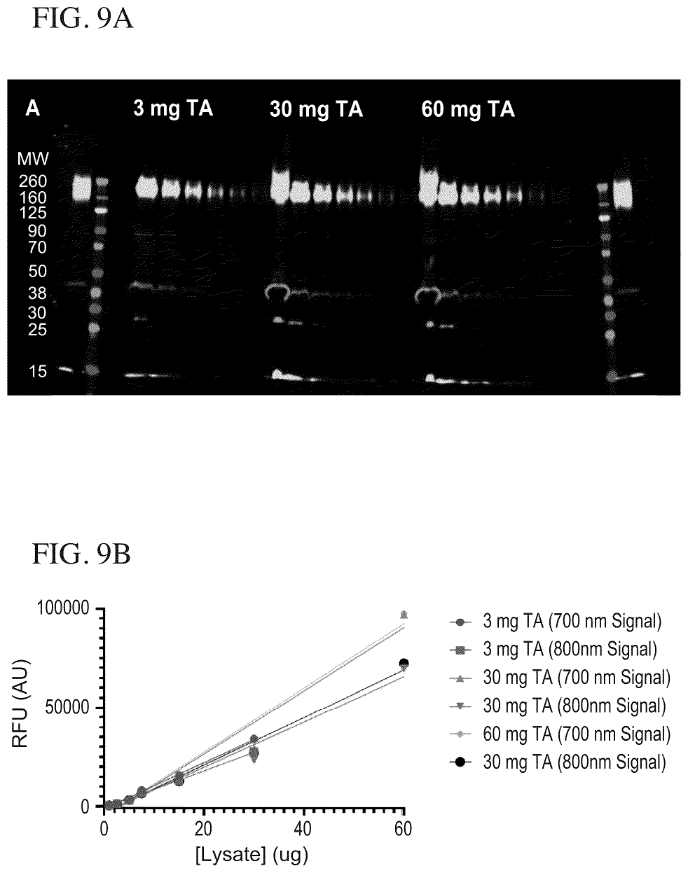

The linear range of the method was determined using a dilution series of healthy control TA. The experiment was performed from lysate generated from three different masses of healthy TA from the same donor. FIG. 9A shows a Western blot merged image showing the dilution series of TA lysate starting from 60 μg to 1 μg. The 3 mg TA tissue preparation had insufficient material to include a 60 μg point. FIG. 9B is a linear plot of the 700 and 800 nm signals from the analysis for the Western blot. Table 3 provides additional details corresponding to FIG. 9B.

TABLE 3
Signal linearity of the αDG assay.

| | 700 nm | 800 nm | 700 nm | 800 nm | 700 nm | 800 nm |
|---|---|---|---|---|---|---|
| Sample | slope | slope | intercept | intercept | r² | r² |
| 3 mg TA | 1168 | 938 | −1371 | −946 | 0.9961 | 0.9963 |
| 30 mg TA | 1597 | 1146 | −5561 | −3271 | 0.9649 | 0.9759 |
| 60 mg TA | 1623 | 1204 | −4970 | −3273 | 0.9808 | 0.9851 |

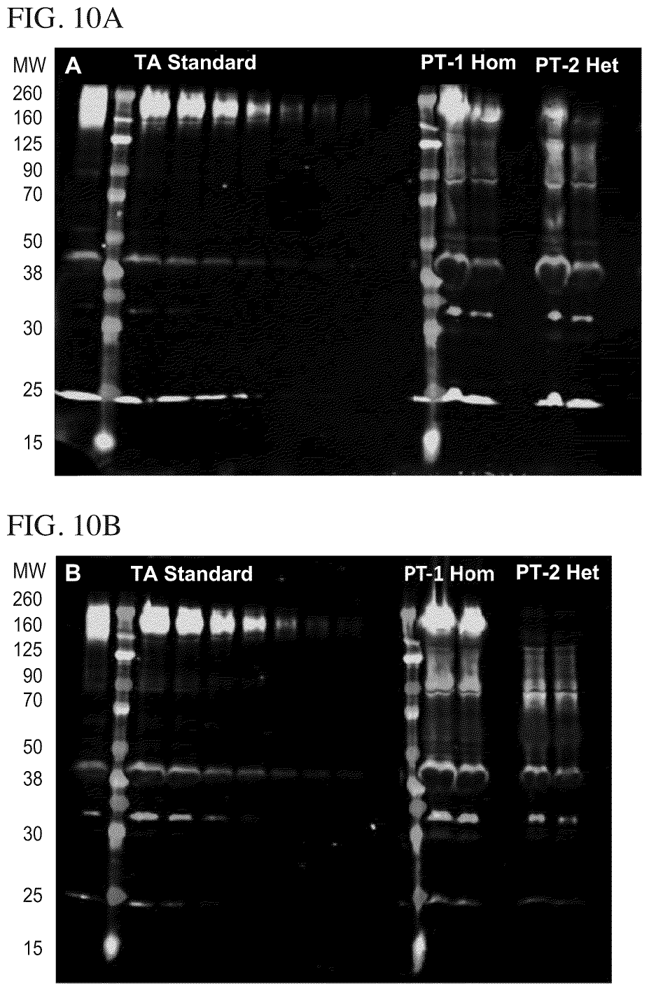

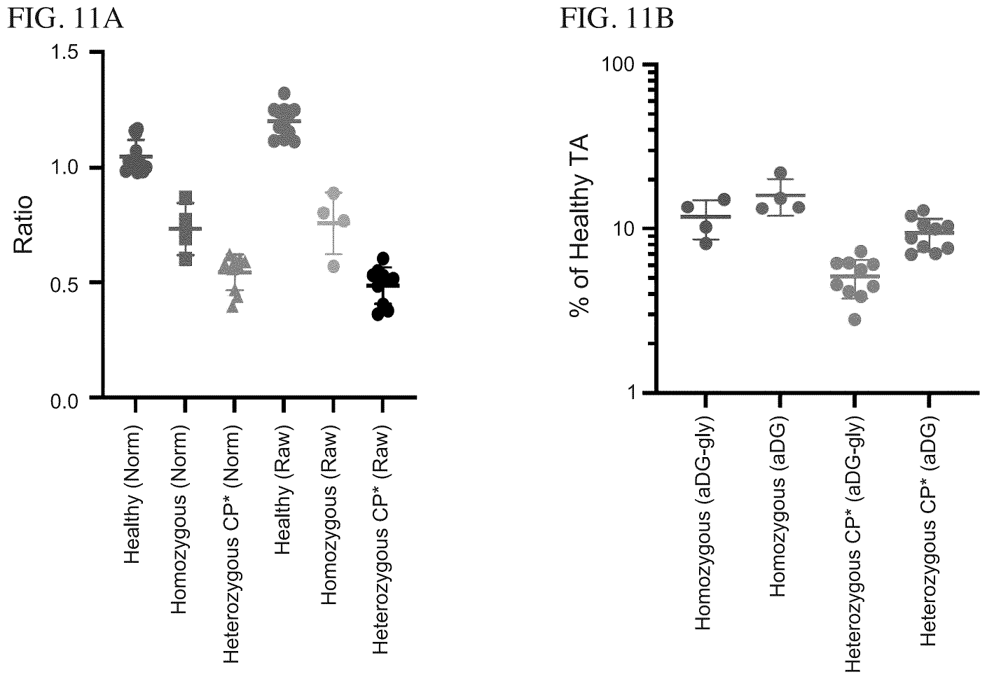

A subset of biopsies from patients enrolled in the natural history study of Example 3 were analyzed using this Western blot method to determine the extent of αDG glycoscylation. Since patient samples are expected to have reduced αDG glycosylation, two concentrations of patient lysate were interrogated to ensure that signals would fall in the linear range of the assay. More compound pathogenic heterozygous patients were included in the testing as these patients are expected to have reduced glycosylation relative to L276I homozygous patients. FIGS. 10A-10B show Western blot merged examples containing patient samples. The TA standard span 25 μg to 0 μg. Patient samples were run at two concentrations of lysate and results of ratio were averaged. As shown in FIGS. 10A-10B, there is reduced staining for the glycosylated form of αDG and there is a significant amount of αDG that is not glycosylated. FIG. 11A shows the normalized ratio determined by interpolation of signal intensities and using the raw signal intensities from integration (700 nm/800 nm). FIG. 11B shows the interpolated amounts of total αDG (αDG) and glycosylated αDG (αDG-gly) for the homozygous and compound heterozygous patients relative to healthy tissue. Heterozygous *CP denotes compound pathogenic heterozygous mutation.

The multiplexed Western blot method described in this example was developed to detect the extent of αDG glycosylation relative to total αDG from muscle biopsy samples and presented as the αDG ratio. The two primary antibodies used showed good specificity for αDG and glycosylated αDG. The detection signals show good linearity with lysate concentration. LGMD2I/R9 patient samples from an ongoing longitudinal natural history study were analyzed and a significant reduction in αDG glycosylation was measured. The compound pathogenic heterozygous patients showed a greater loss in glycosylation content than homozygotes. This finding aligns with known differences in disease onset and progression for compound pathogenic heterozygotes who generally exhibit an earlier disease onset and more rapid progression than homozygotes. This method may prove useful in studies of LGMD2I/R9 where status of glycosylation may serve as a primary disease marker.

### Example 3: Longitudinal Measurement Using an Innovative Western Blot Assay of Glycosylation of Alpha-Dystroglycan (αDG) in Patients with Limb-Girdle Muscular Dystrophy Type 2i (LGMD2I/R9)

Introduction: Limb-girdle Muscular Dystrophy (LGMD) Type 2i, also called LGMDR9 FKRP-related, is caused by bi-allelic partial loss-of-function of the fukutin-related protein (FKRP) gene. The FKRP enzyme performs a critical step in the glycosylation of alpha dystroglycan (αDG); impaired FKRP enzyme activity leads to the formation of dysfunctional hypoglycosylated αDG, resulting in chronic myocyte injury and muscular dystrophy.

Objective: A natural history and biomarker development study was carried out in patients with LGMD2I/R9 to assess appropriate clinical outcome assessments (COAs) and explore prognostic biomarkers for LGMD2I/R9 to facilitate clinical trial development for potential therapeutics.

Methods: This study is an ongoing, prospective, 12-month observational study of clinically affected and genetically defined LGMD2I participants aged 10 to 65 years enrolled at 11 international academic centers. Tibialis anterior muscle biopsies were obtained at baseline, month 6, and month 12 for evaluation of glycosylated αDG levels by a Western blot assay (described below and in greater detail in Example 5).

Results: To date, 89 participants have completed 12 months of follow up and 57% were women. Overall, participants were a mean age of 37 years, mean symptom onset at 16.6 years and mean duration since diagnosis of 10.4 years. 73% were homozygous for the c.826C>A (p.L276I) founder mutation. Data from 71 individuals with LGMD2I showed reduced levels of glycosylated αDG (median 8.5% of healthy). Glycosylation of αDG appears to mirror the severity of LGMD2I disease, with other (non-L276I/L276I) FKRP genotypes showing reduced glycosylated αDG protein levels (median 4.6% of healthy) compared to L276I/L276I homozygotes (median 10.5% of healthy).

Conclusions: Data from a natural history study of patients with LGMD2I suggest that the innovative Western blot assay can be used to evaluate glycosylation of αDG, which is central to the pathogenesis of LGMD2I. Measurement of αDG glycosylation may provide a relevant approach to assess the impact of potential therapies for LGMD2I.

### Introduction

Limb Girdle Muscular Dystrophy Type 2I (LMGD2I) is an autosomal recessive disease of striated muscle caused by mutations in the fukutin-related protein gene, FKRP, which codes for the glycosyltransferase enzyme that is critical for the glycosylation of alpha-dystroglycan (αDG). The heavily glycosylated αDG is a component of the dystrophin-glycoprotein complex that anchors the intracellular cytoskeleton of muscle cells to the extracellular matrix through interactions of the matriglycan on αDG.

In LGMD2I, αDG is hypo-glycosylated due to partial loss of function mutations in FKRP making muscle cells susceptible to contraction induced injury that results in inflammation, fibrosis, and fatty infiltrate leading to muscle wasting and impaired function. Currently, there is no approved treatment available for LGMD2I, however, BBP-418 (ribitol) has been shown to increase alpha-DG glycosylation in LGMD2I mouse models.

To support our on ongoing development of BBP-418 as a potential therapy for LGMDI2I, we sought to identify a biomarker that could reflect the glycosylation state of αDG, as hypo glycosylation of αDG is the single causal pathway of this disease. BBP-418 increases the intracellular concentration of CDP-Ribitol helping to drive residual activity of the mutant FKRP enzyme and increasing αDG glycosylation levels. Hence, we rationalized that measurement of glycosylated αDG levels would be a viable biomarker strategy in LGMD2I.

### Methods

Tissue Lysis and sample preparation: Tissue samples were processed in lysis buffer, supplemented with protease inhibitors. Total protein in lysate was determined by BCA (Thermo Fisher) for RIPA and protein 660 kit (Thermo Fisher) for SDS-Urea and tested at 60 μg for CP Heterozygous samples and 40 μg for Homozygous samples. Differential protein loading is used for different patients to compensate for patient deficits in glycosylated αDG. Patients with LGMD2I/R9 inherently have lower levels of protein in muscles, which makes it difficult to detect via prior iterations of this assay, including the version described in Example 2 herein. Loading additional protein helps compensate for this deficiency and allows the assay to detect lower-level ranges. This is further stratified by patient genotype: more severe patients require greater protein loading.

Western Blotting: For detection of target proteins αDG-Total and αDG-Glycan, membrane was probed simultaneously with primary antibodies AF6868 alpha Dystroglycan (R&D Systems) and IIH6C4 alpha Dystroglycan (Millipore), followed by fluorescent secondary antibodies IR800CW Mouse Anti-Sheep and IR680 Goat Anti-Mouse secondary antibodies (LiCor). The membrane was visualized in Odyssey CLx imager (LiCor) and intensities of the 700 (αDG-Glycan, red bands) and 800 (αDG-Total, green bands) nm channels were recorded. Merged channel resulted in yellow bands when glycosylated and total αDG signals overlap. Notably, prior iterations of this assay, including that described in Example 2 herein, analyzed signal rather than total amounts of protein. Here, amounts of glycosylated and total αDG are determined using standard curves, and an interpolated ratio is computed by dividing the amount of glycosylated αDG to the amount of total αDG.

Data Analysis: Integration boxes were drawn using Image Studio software with specific dimensions over molecular weight range for standards, test articles and control samples. Data analysis was done in Excel (Microsoft). The 700 nm and 800 nm raw signals of test articles were interpolated to healthy control TA standard curve using regression to a quadratic equation, to give relative healthy TA amounts of glycosylated and total αDG. Healthy positive control (HPC) at 10 μg was deployed to monitor assay performance. Notably, prior iterations of this assay, including that described in Example 2 herein, utilized a linear fit rather than a quadratic fit. The quadratic fit employed here covers a larger range of concentrations and permits extension of the range of detectable signals. The quadratic fit also allows more sensitive detection of baseline measures, enabling detection of lower signal levels. This also allows for patient identification and stratification of homo-/hetero-zygous subjects.

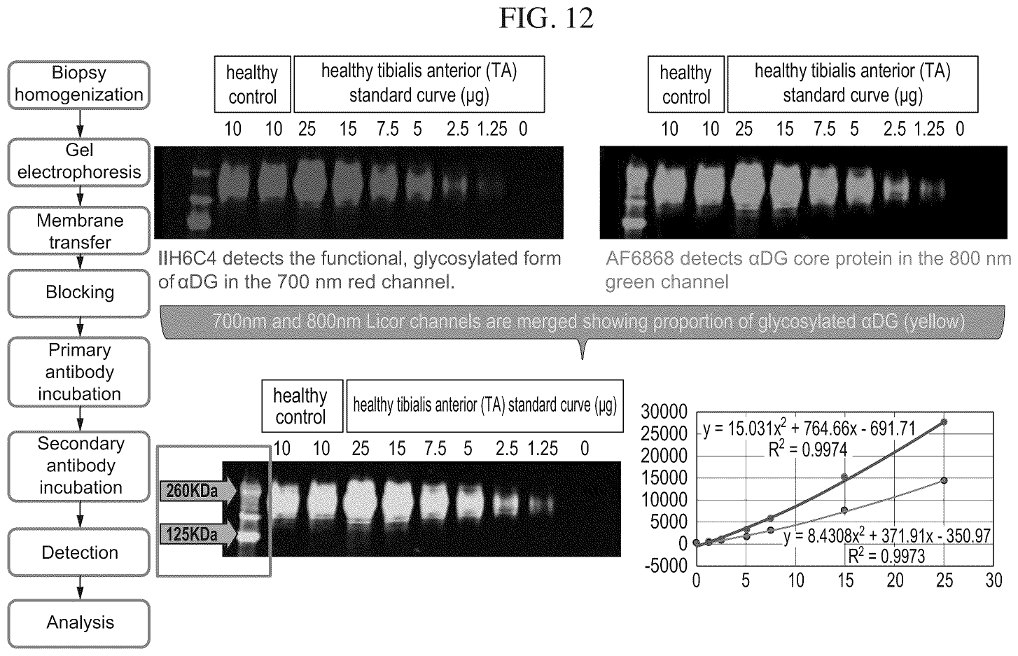

FIG. 12 schematically illustrates a workflow for the Western Blot method. Briefly, biopsies are collected from a subject at a desired timepoint and homogenized. Gel electrophoresis is performed, followed by membrane transfer and blocking. Sample is incubated with the primary antibody followed by the secondary antibody. Detection and analysis are subsequently performed. Notably, only the 125-260 kDa region is analyzed. A broader region of 70-260 kDa was used in prior iterations of the assay, including that described in Example 2 herein. The use of a narrower range of interest for the assay eliminates static and/or unnecessary data and leads to cleaner results.

Multiple sections of a single healthy donor TA biopsy sample are homogenized and the resulting lysate is pooled together and stored in single use aliquots at −80° C. These aliquots are used to generate a standard curve which is used in every assay. In addition, a quality control of 10 μg lysate is used to assess assay performance by measuring interpolatability to the standard curve.

Example 5 provides additional details relating to the Western Blot assay.

### Results

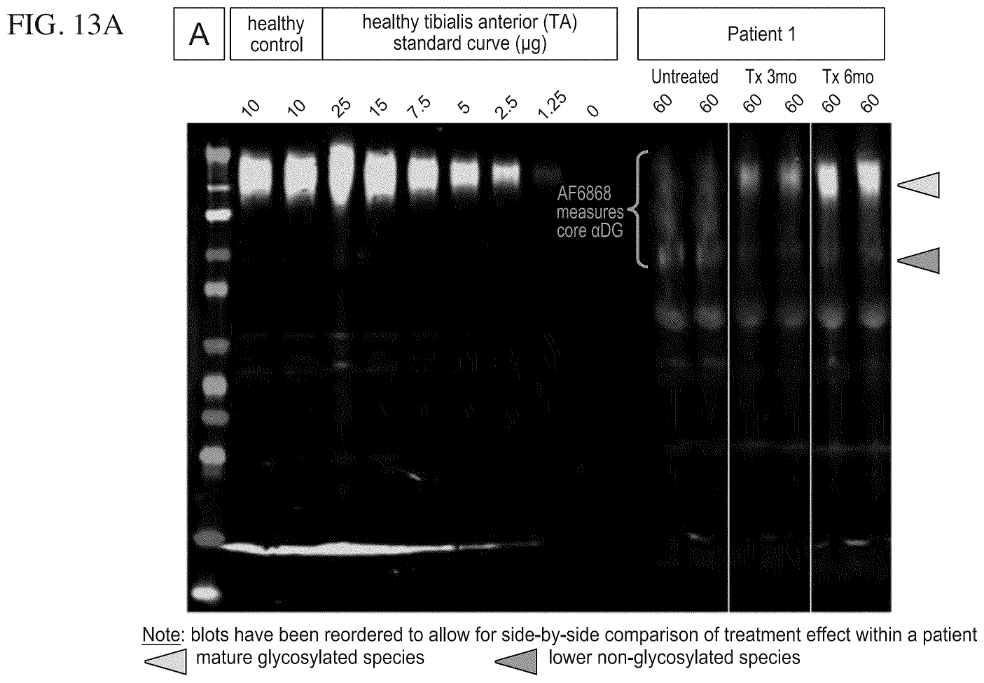

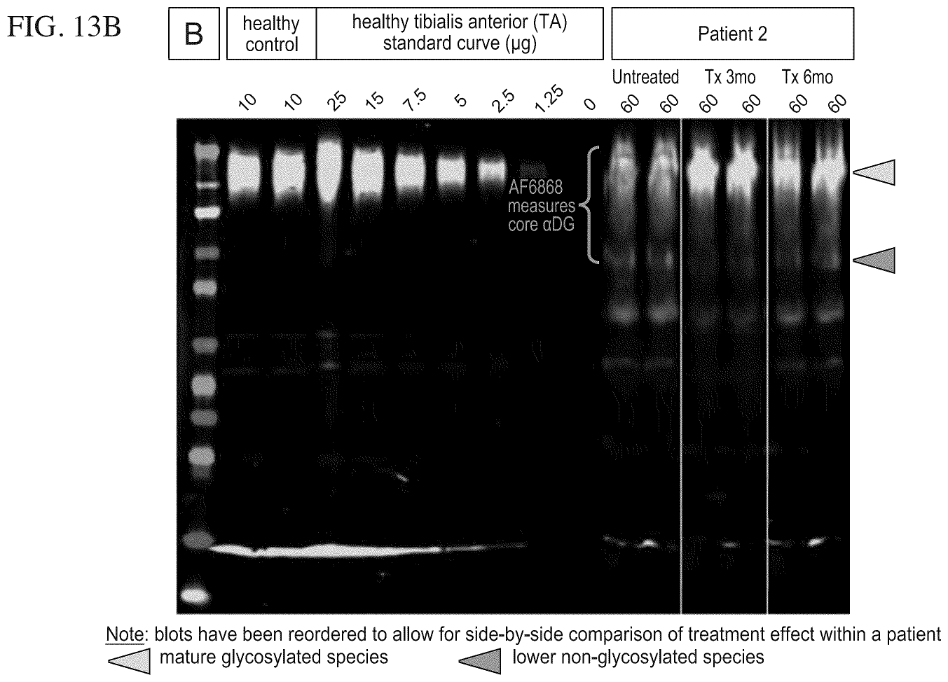

FIGS. 13A-13B show that treatment with ribitol dramatically increases αDG glycosylation in LGMD2I/R9 muscle biopsies. Notably, due to low levels of glycosylated αDG at baseline (compared to healthy individuals), LGMD2I/R9 patient samples require higher protein loading to detect glycosylated αDG levels.

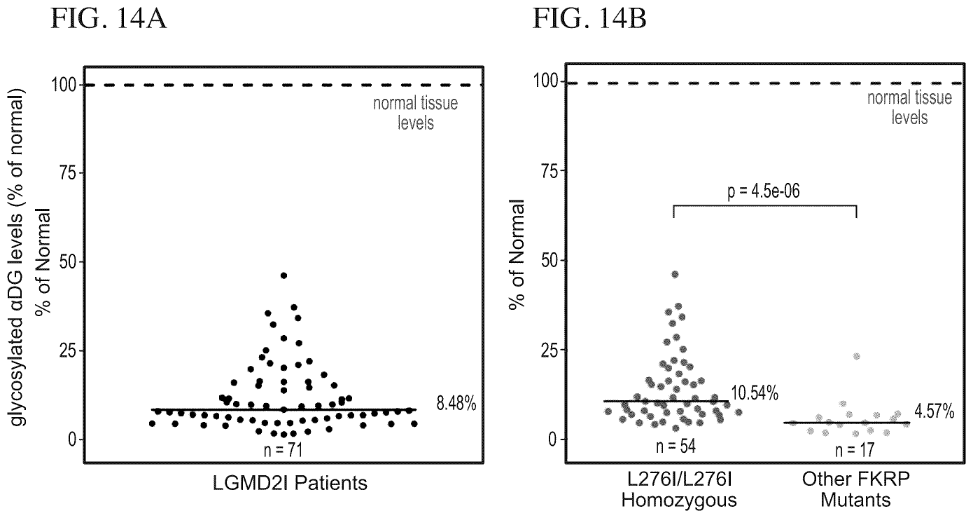

FIGS. 14A-14B show that LGMD2I/R9 patients have less functional, glycosylated αDG relative to healthy subjects. Samples are taken from tibialis anterior (TA) muscles. Bars denote median values; Wilcoxon test was used to calculate p values.

FIG. 15 shows that glycosylated αDG levels remain stable over time. Glycosylated αDG levels remain stable at a second visit (e.g., 6, 9, or 12 months after a first visit) compared to a first visit in untreated LGMD2I/R9 patients. The stability of this measure supports the ability of the assay to detect differences with treatment.

### Conclusions

Preliminary data from a natural history study of patients with LGMD2I suggest that an innovatie Western blot assay can be used to evaluate glycosylation of αDG, a measure of the prognostic biomarker reflecting the root cause of LGMD2I/R9. A clear difference in glycosylated αDG content in patients with LGMD2I/R9 relative to normal control tissue is observed. Additionally, glycosylated αDG content reflects the LGMD2I/R9 genotype: Other (non-L2761 homozygous) patients showed a greater loss in glycosylation than L276I homozygous patients. This observation aligns with known differences in disease onset and progression as these patients exhibit an earlier disease onset and more rapid progression than L276I homozygous patients. These natural history data suggest that longitudinal measurement of glycosylated αDG remains stable over 6-12 months.

This innovative, validated assay methodology utilized to quantitate αDG levels is similar to an assay recently published for quantitation of microdystrophin (Soderstrom et al. 2023) from muscle biopsies.

Measurement of αDG glycosylation may provide a relevant approach to assess the impact of potential therapies for LGMD2I.

### Example 4: Preliminary Results from an Open Label Phase 2 Study of Ribitol in Patients with Limb-Girdle Muscular Dystrophy Type 2i (LGMD2I/R9)

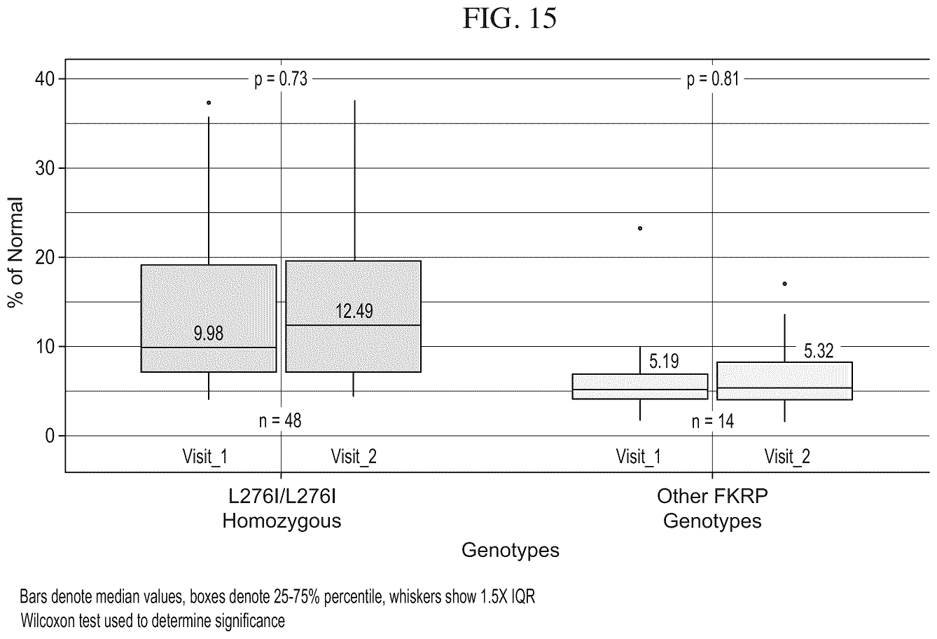

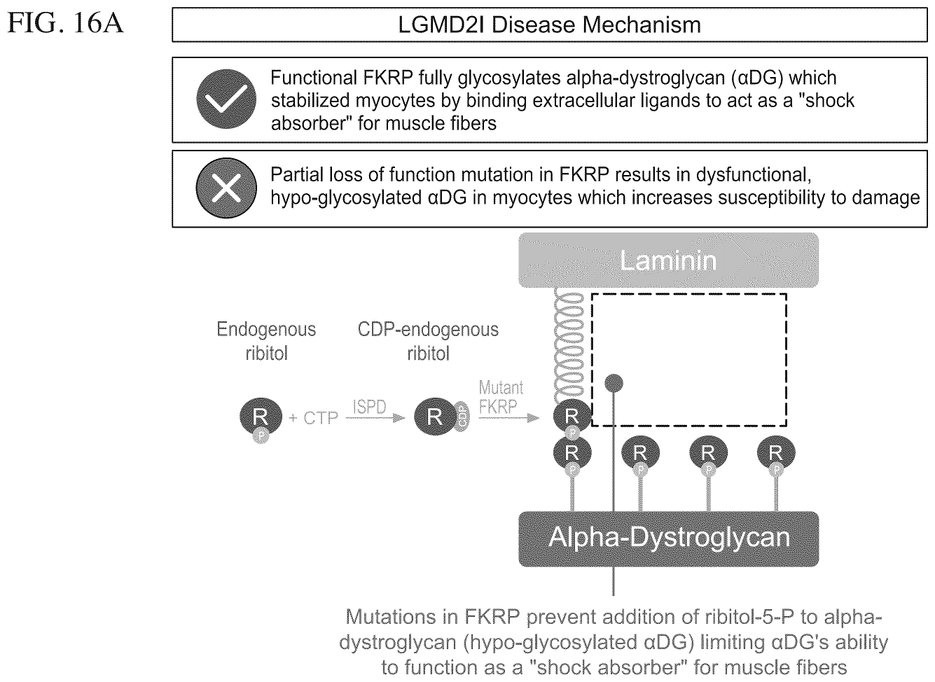

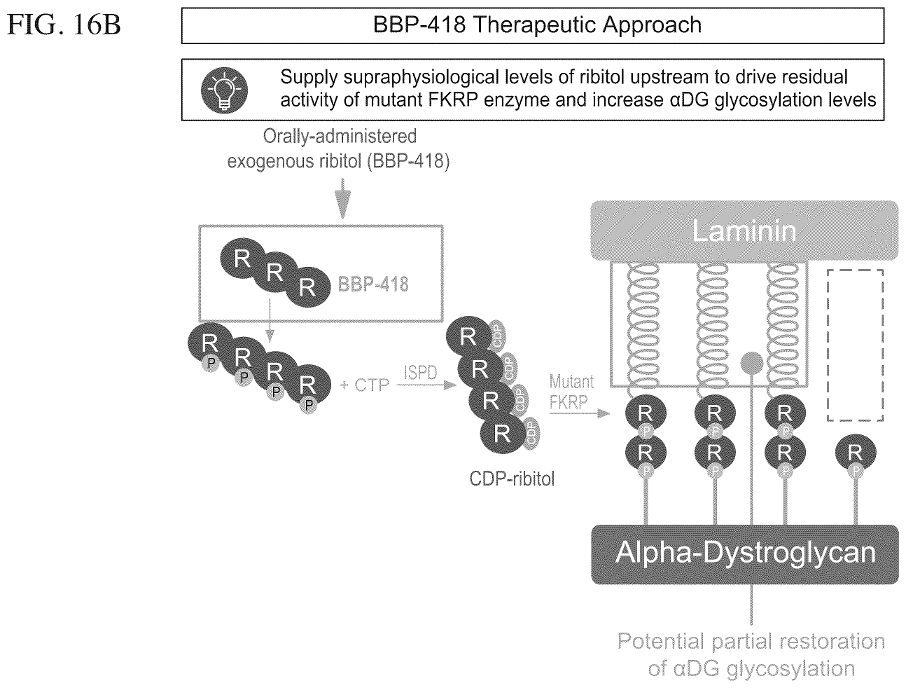

Introduction: Limb-girdle Muscular Dystrophy (LGMD) Type 2i, also called LGMDR9 FKRP-related, is caused by bi-allelic (e.g., autosomal recessive) partial loss-of-function of the fukutin-related protein (FKRP) gene, which results in hypoglycosylation of alpha-dystroglycan (αDG). Ribitol (BBP-418) is an orally administered investigational substrate supplementation therapy intended to saturate the partially functional FKRP enzyme, driving increased glycosylation of αDG, and potentially ameliorating the root cause of LGMD2I/R9. FIG. 16A-16B schematically illustrate the LGMD2I/R9 disease mechanism (FIG. 16A) and therapeutic approach (FIG. 16B).

Objectives: The Phase 2 study is intended to explore the safety and tolerability of ribitol. Additionally, the feasibility and usefulness of clinical efficacy and pharmacodynamic (PD) assessments in patients with LGMD2I receiving ascending doses of ribitol will be investigated.

Methods: This study is an open label dose escalation study of patients with LGMD2I/R9. Part 1 of the study involved three dose cohorts (6 g QD, 6 g BID and 12 g BID) treated for 3 months. During Parts 2 (target maximal dose) and 3 (ongoing OLE), all patients received 12 g BID, dose adjusted for lower weight.

Results: 14 patients with LGMD2I/R9 (aged 12-53, 8/14 homozygous for the L276I/L276I mutation) were enrolled. After 90 days of treatment, participants showed an approximate doubling of glycosylated αDG which was sustained over 15 months of treatment. A sustained reduction in serum creatine kinase (CK) of >75% was also detected over 15 months. Following 15 months of ribitol treatment, improvements in NSAD, 100MTT and 10MWT velocity were observed. This compares favorably to natural history data showing decline over the 12-month interval prior to treatment with ribitol. Across a range of ribitol dose levels no observed treatment-related serious adverse events, dose limiting toxicities or discontinuations were observed.

Conclusions: Preliminary Phase 2 data from patients with LGMD2I/R9 suggest a positive effect of ribitol on αDG glycosylation, CK, NSAD, 10MWT velocity and 100MTT. A global, double-blind placebo-controlled Phase 3 study is planned.

### Methods

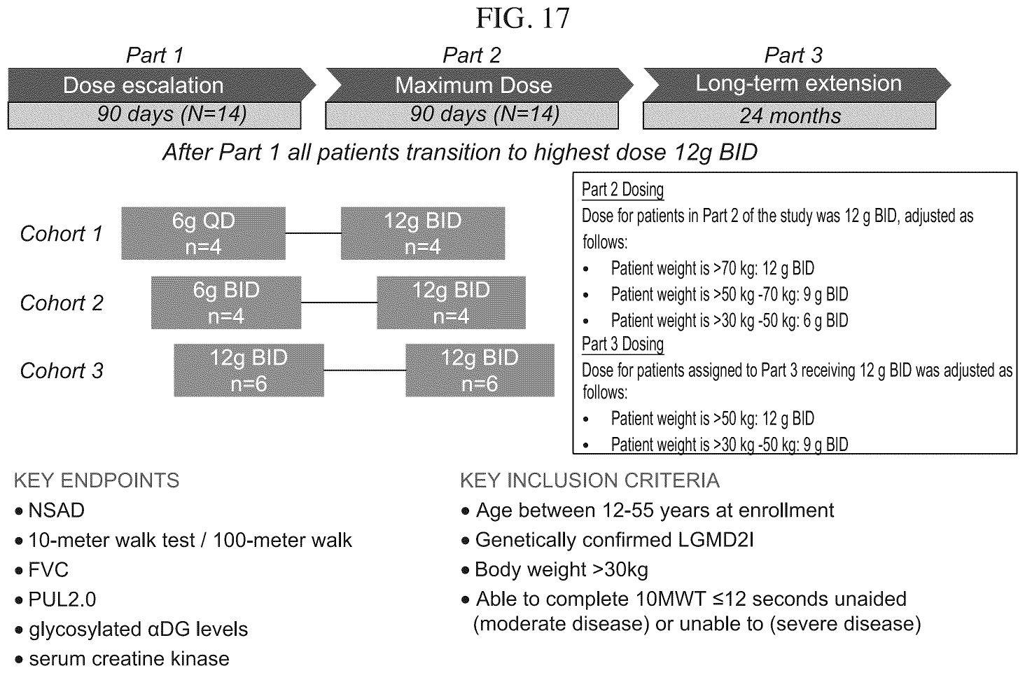

FIG. 17 schematically illustrates the details of the clinical study. Briefly, the study includes 3 parts: a 90 day dose escalation phase (Part 1), a 90 day maximum dose phase (Part 2), and a 24 month long term extension phase (Part 3). In Part 1, 14 total patients are studied across three cohorts (6 g QD, 6 g BID, and 12 g BID ribitol). After Part 1, all patients transition to the highest dose, 12 g BID ribitol. Dosing adjustments in Part 2 were as follows: for patients with weight >70 kg: 12 g BID; for patients with weight between 50-70 kg: 9 g BID; and for patients with weight between 30-50 kg: 6 g BID. Dosing adjustments in Part 3 were as follows: for patients with weight >50 kg: 12 g BID; and for patients with weight between 30-50 kg: 9 g BID.

Key inclusion criteria for the study include age between 12-55 years at enrollment; genetically confirmed LGMD2I/R9 diagnosis; body weight of at least 30 kg; and ability to complete a 10 minute walk test in <12 seconds unaided for moderate disease, or unable to complete the 10MWT for severe disease.

Key endpoints for the study include NSAD; 10 or 100 meter walk test; FVC; PUL2.0; glycosylated αDG levels; and serum creatine kinase (CK).

### Results

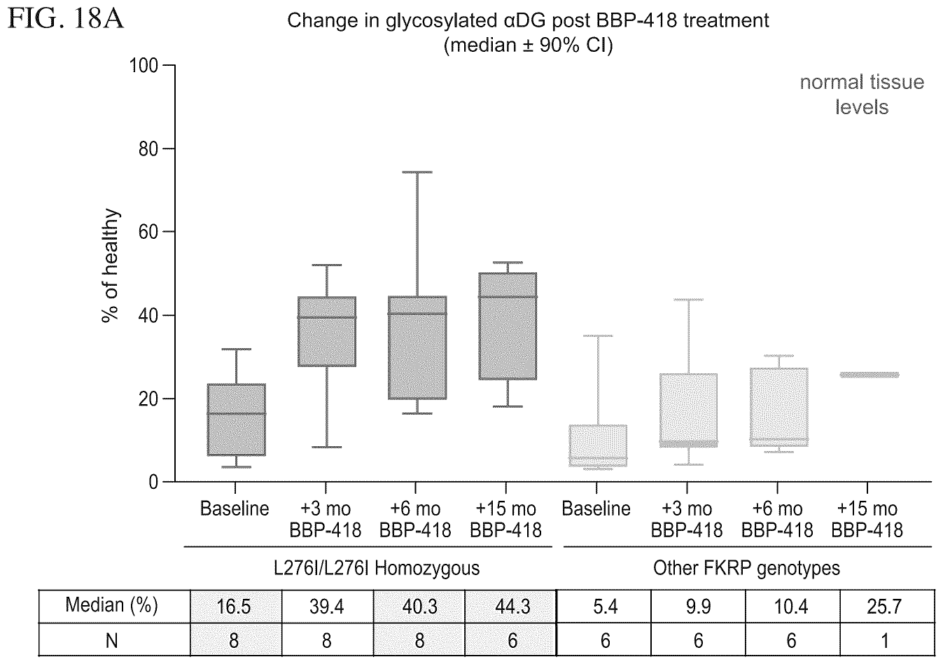

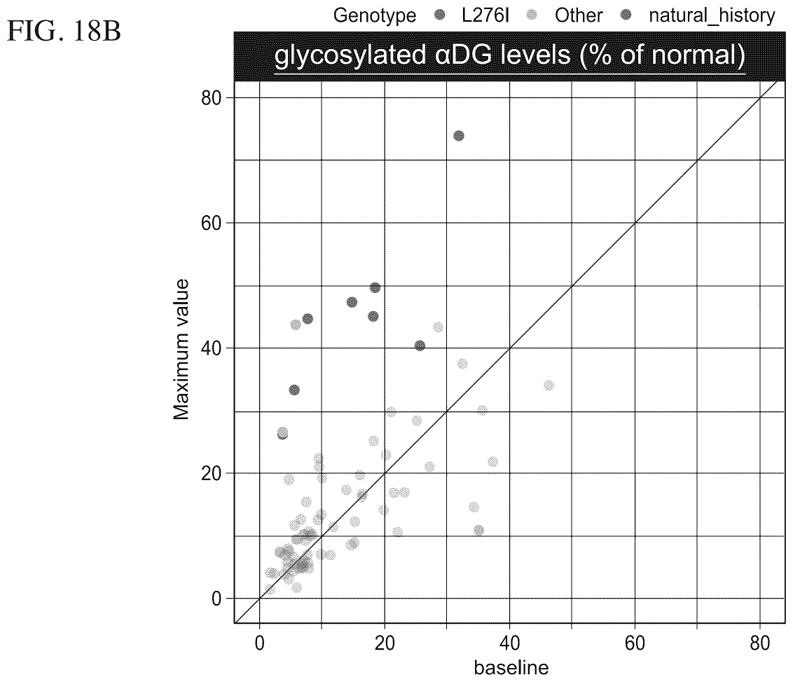

FIGS. 18A-18B demonstrate the effects of ribitol treatment on glycosylated αDG levels compared to those observed in the natural history described in Example 3. FIG. 18A shows the change in glycosylated αDG post-ribitol treatment (median±90% confidence interval (CI)).

Patient samples were interpolated to a standard curve to determine % normal glycosylation of αDG Median and 10-90% percentile are shown; the Wilcoxon test was used to determine significance. 1 patient missed their 6-month biopsy, and a 9-month biopsy was obtained, this was included in month 6 results.

FIG. 18B shows glycosylated αDG levels as % of normal. Glycosylated αDG levels were approximately double with ribitol treatment vs. baseline values. Increases in glycosylated αDG on treatment are markedly different from natural history study in which αDG are stable over time.

Armit plot comparing baseline % glycosylated αDG levels versus their maximum value over 6-12 months untreated (grey) or on treatment (green). For natural history samples, visit 2 visit 2 data are shown; for Phase 2 samples, maximum value is shown.

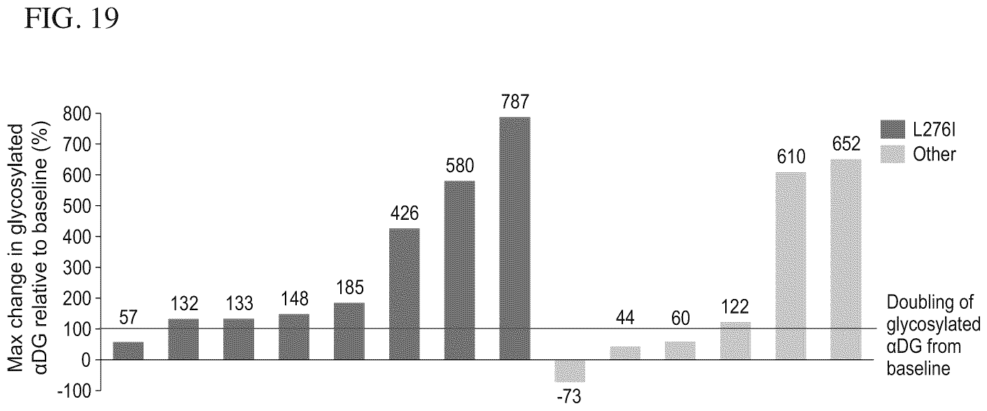

FIG. 19 show the maximum change in glycosylated αDG relative to baseline observed for individual patients. 13 of 14 patients showed increased glycosylated αDG levels with ribitol treatment vs. pre-treatment baseline levels. 10 of 14 patients showed a ≥100% increase, or a doubling, in glycosylated αDG levels relative to their pre-treatment levels.

Patient samples were interpolated to a standard curve to determine % normal glycosylation of αDG. Max percent change in glycosylated αDG levels over 3-15 months of BBP-418 treatment relative to baseline is shown. Blue line indicates doubling of glycosylated αDG from baseline.

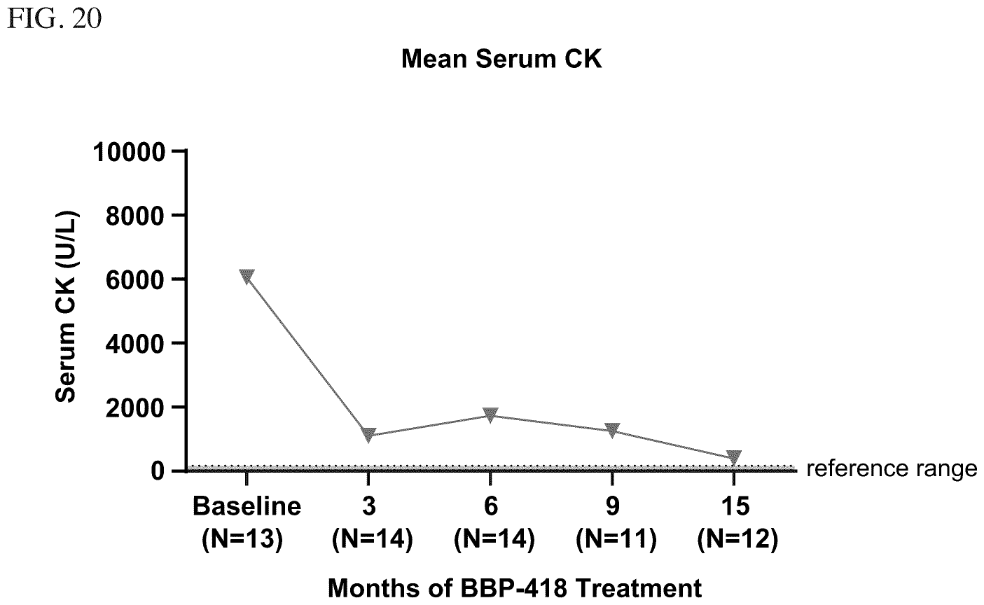

FIG. 20 shows the change in serum creatine kinase, a biomarker of muscle damage, after 15 months of treatment with ribitol. A notable change in serum CK is observed after even 3 months of treatment.

Cohort 1 day 1 CK draws taken after functional assessments; all other draws done prior to functional assessment. After Day 90, all subjects received 12 g BID (weight-adjusted). Reference range for CK is 55-170 unites/L for men and 30-135 unit/L for women, figure shows reference range from 30-170 units/L.

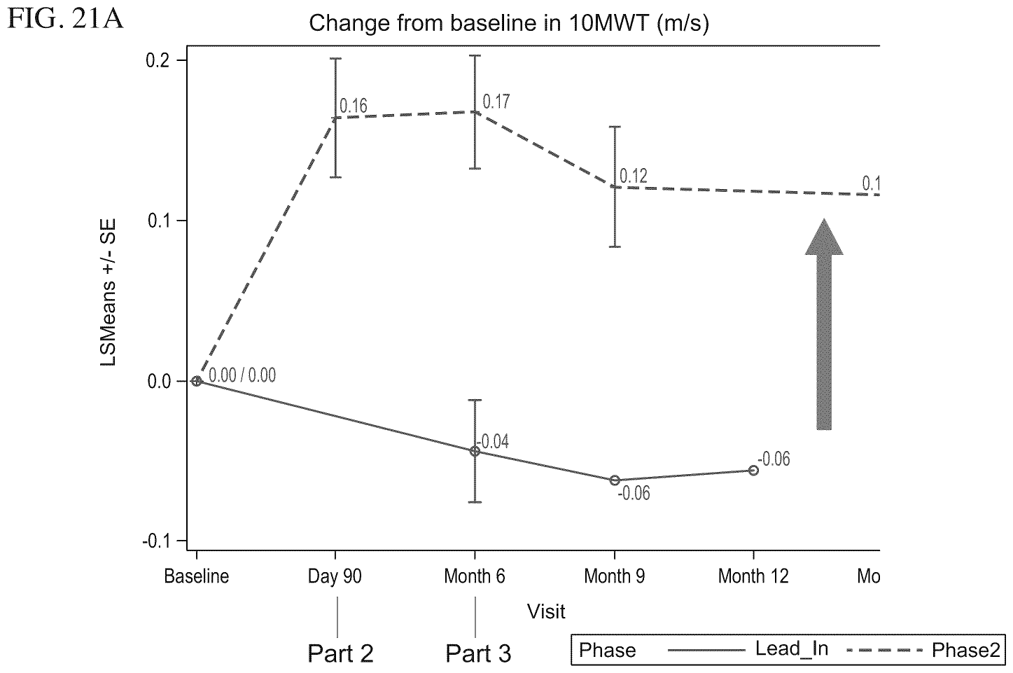

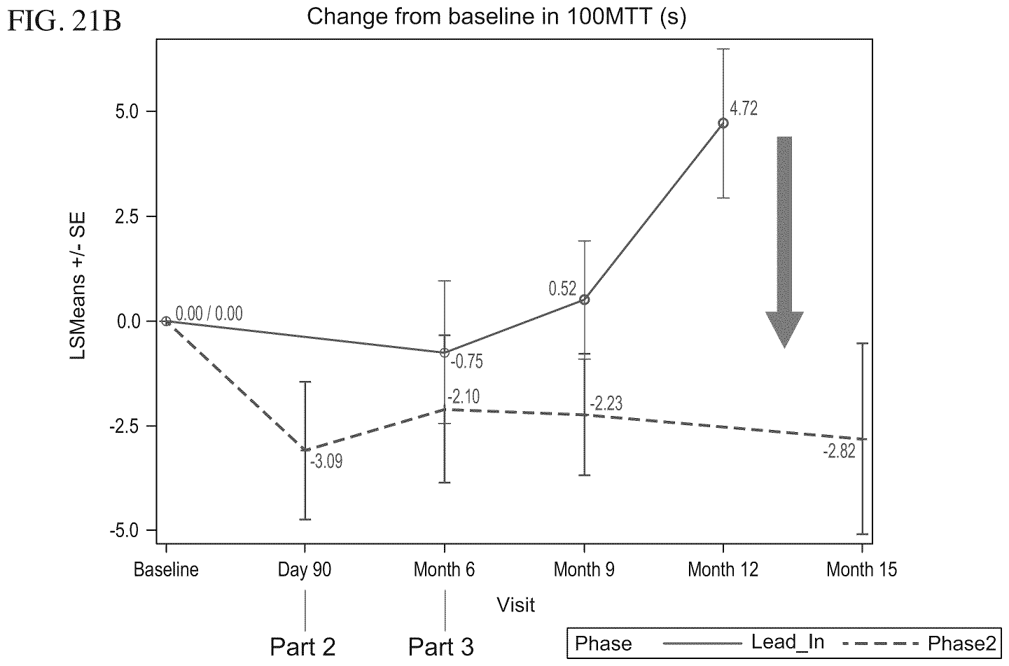

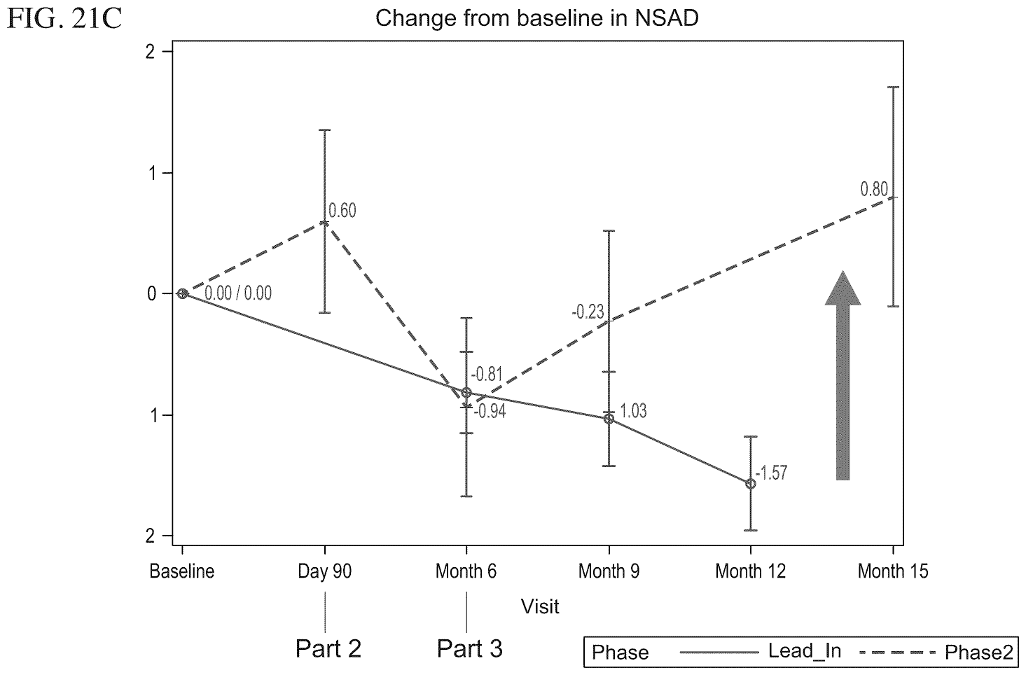

FIGS. 21A-21C show the improvements in ambulatory and clinical measures including change from baseline in 10MWT (FIG. 21A), change from baseline in 100MTT (FIG. 21B), and change from baseline in NSAD (FIG. 21C) after 15 months of treatment with ribitol. Solid lines denote natural history data collected prior to Phase 2 enrollment (see Example 2) while dashed lines denote on-treatment data collected during the Phase 2 study from the same patient population.

Ribitol was well-tolerated over 15 months of treatment. During the course of the study, 136 adverse events were recorded, with 12 possibly or probably related to ribitol treatment. The 14 possibly or probably treatment-related AEs included diarrhea (6 incidents, 75% grade 1, 25% grade 2), dehydration (1 incident, grade 1), nausea (2 incidents, both grade 1), vomiting (2 incidents, both grade 1), dyspepsia (1 incident, grade 1), gastroenteritis (1 incident, grade 2), and headaches (1 incident, grade 2). 3 severe adverse events unrelated to the treatment were recorded. No adverse event led to a discontinuation or interruption in therapy.

### Conclusions

Ribitol supplementation therapy provides supraphysiological levels of ribitol upstream of the mutant FKRP enzyme to drive residual activity of the enzyme and increase levels of glycosylated αDG. Ribitol treatment increased levels of glycosylated αDG at 3 months in a Phase 2 study, which was sustained over time (15 months). Approximate doubling of glycosylated αDG was observed in both L276I homozygous and other FKRP genotype LGMD2I/R9 patients. Consistent with the changes in glycosylated αDG observed, a sustained reduction in serum CK of >75% was observed over 15 months of treatment. An improvement in NSAD, 10MWT velocity, and 100MTT was observed with 15 months of ribitol treatment. Ribitol was well tolerated with only minor GI adverse events.

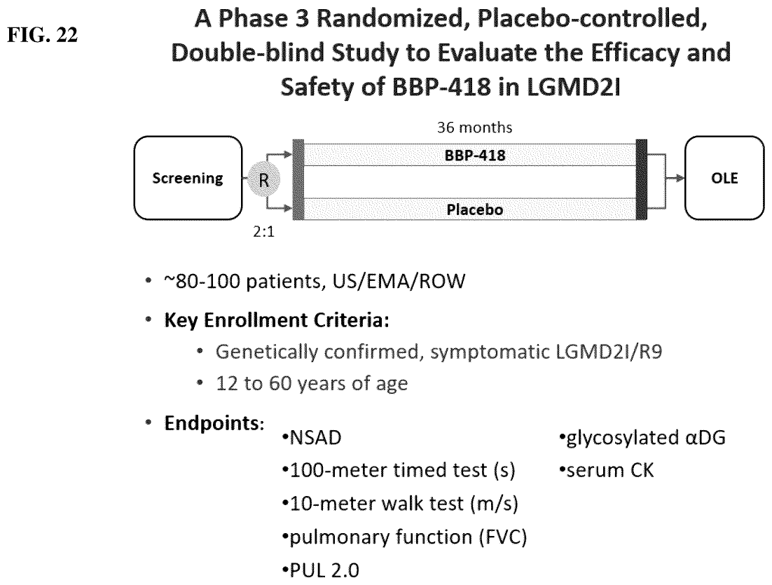

Based on the encouraging data from a Phase 2 study (MLB-01-003), a global, double-blind placebo-controlled Phase 3 study is planned. Details of the planned Phase 3 study are included in FIG. 22.

### Example 5: Validation of an Innovative Western Blot Assay to Monitor Patterns and Levels of Alpha-dystroglycan in Skeletal Muscle of Patients with Limb Girdle Muscular Dystrophies

Alpha-dystroglycan (αDG) is a highly glycosylated peripheral membrane protein which is an integral part of the dystrophin-glycoprotein complex (Nickolls and Bonnemann 2018; Barresi and Campbell 2006; Le et al. 2018; Han et al. 2009). It is encoded by the DAG1 gene and is ubiquitously expressed. The DAG1 gene translates into a single polypeptide which is then cleaved into two subunits: αDG and βDG. αDG is secreted into the extracellular space but interacts non-covalently with βDG, which remains anchored in the membrane (Kanagawa et al. 2004). αDG is found in cells of various tissues including striated muscle, nervous system, digestive tract, kidney, skin, and reproductive organs where it provides a crucial link between the cytoskeleton, through its indirect binding to dystrophin, and the basal lamina. In skeletal muscles, αDG interacts with extracellular ligands, including laminin-α2, a significant functional protein of the extracellular matrix crucial for muscle function and structure (Balci-Hayta et al. 2018). This interaction confers structural stability to the sarcolemma during contraction. αDG has a mucin domain rich in O-linked glycans, such that carbohydrates constitute over half of the glycoprotein's mass (Kanagawa et al. 2004). In this heavily glycosylated mucin site, 3 specific threonine sites, T317, T319, and T379, are modified for matriglycan synthesis (Sheikh et al. 2022; Inamori et al. 2012). Reduced levels of matriglycan, result from mutations in at least seventeen genes causing a variety of complex diseases affecting both muscular and nervous systems (Yoshida-Moriguchi and Campbell 2015; Hewitt 2009; Barresi and Campbell 2006; Martin 2005).

The Western blot assay described herein was developed to monitor changes in αDG in the skeletal muscle. The assay is based on multiplexed detection using fluorescence. For detection the monoclonal antibody IIH6C4 that recognizes a specific functional epitope of the matriglycan chain of glycosylated αDG (a tandem repeat of xylose and glucuronic acid was used; the matriglycan is the specific recognition site for laminin α2 (Ervasti and Campbell 1993). Therefore, IIH6 binding uniquely acts as a proxy for molecular functional assessment of αDG. Specifically, the presence of 16-reactive matriglycan on αDG indicates that the glycosylation pathway is intact, and that the protein can interact with the extracellular matrix, thereby establishing the clinical relevance of this antibody (Stevens et al. 2013). In addition, the core protein is detected using AF6868, regardless of its glycosylation status. This approach of simultaneous detection of both core and glycosylated protein from the same sample interrogates both the amount and status of glycosylation of αDG. The assay incorporates a calibration curve using normal donor TA for determination of relative amounts of αDG. Additionally, quality controls are used to monitor the assay's performance. The precision, linearity, specificity, sensitivity, and consistency in detection of alterations in αDG levels are reported. In addition to the quantitative aspect, this has the potential to provide information on molecular changes the protein undergoes over time or upon therapeutic intervention in patients with α-dystroglycanopathies.

The applicability of the assay was demonstrated using skeletal biopsy samples from patients with LGMD2I/R9, a disease caused by mutations in fukutin-related protein gene (FKRP). FKRP is a glycosyltransferase that adds a critical ribitol 5-phosphate to the growing matriglycan chain in α-DG during its functional maturation. FKRP's role in α-DG glycosylation is significant in brain and striated muscles functions and mutations can disrupt this process, leading to various forms of muscular dystrophy. Overall, this bioassay and the use of glycosylated αDG as a potential biomarker serve as a powerful tool to monitor the efficiency of therapeutic intervention and longitudinal responses to disease-modifying therapies in α-dystroglycanopathies.

### Materials and Methods

Antibodies and Purified Protein: To detect both glycosylated and core αDG, anti-αDG clone IIH6C4 (Sigma, St. Louis, MO) and anti-human DG AF6868 (R&D System, Minneapolis, MN) primary antibodies were used, respectively For secondary antibodies IRDye 680 linked Goat anti-Ms IgM (LI-COR) and Alexa Fluor 790 linked Ms anti-Sheep (Jackson Immuno Research, PA) were used for detecting IIH6C4 and AF6868 respectively. Chameloeon Duo (LI-COR) was used as the molecular weight marker.

Tibialis anterior (TA) biopsy collection: Biopsies from patients with an LGMD2I/R9 diagnosis were collected using fine needle aspiration (FNA) (14-gauge×6 cm; SuperCore™ Instrument; Cincinnati, OH, USA), a minimally invasive biopsy technique, during an Institutional Review Board (IRB) approved Natural History Study (MLB-01-001; NCT04202627). This study was conducted in accordance with the principles laid out in the Declaration of Helsinki. All procedures involving human subjects were performed with respect for their rights and dignity. Written and oral informed consent was obtained from participants, parents, or legal guardians before enrollment into this study, and their anonymity and confidentiality were preserved throughout the study. Up to three serial biopsy cores were obtained per visit and pooled for analysis. For normal control, biopsies from six different normal individuals, without muscular dystrophy, cardiac disease, or diabetes, were obtained from BioIVT (Westbury, NY, USA).

Sample homogenization & considerations for high molecular weight protein extraction: For homogenization, multiple cores of frozen TA biopsies were combined in a 2-mL tube (TissueLyser-safe). A lysis buffer containing 125 mM Tris, 10% glycerol, 10% β-mercaptoethanol, urea, SDS and 1 mM of the protease inhibitor AEBSF was then added to the tissue, along with a single stainless-steel bead. The sample tubes were then processed using a TissueLyser II (Qiagen, Venlo, Netherlands). For storage, to maintain protein stability over time, excess lysate was divided into multiple aliquots and flash frozen at −80° C. to minimize freeze/thaw cycles. Ongoing studies have shown that the protein is stable for at least 18 months.

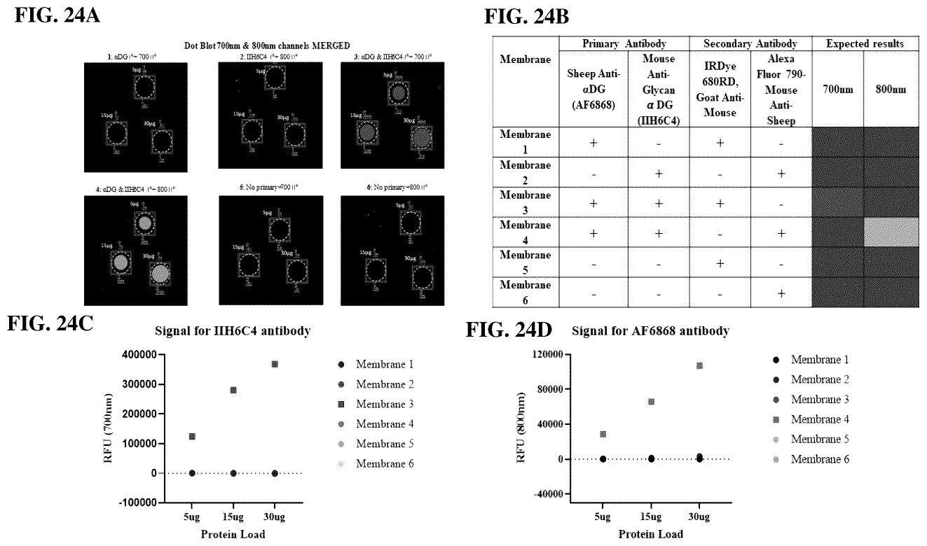

Dot blot assay: The specificity and cross-reactivity of both primary and secondary antibodies were tested using a dot blot assay. Briefly, TA lysate was prepared at three different concentrations: 30, 15, and 5 μg of total protein in 1×PBS. The diluted lysate was drop-pipetted onto six separate nitrocellulose membranes following the same layout and dried at room temperature followed by a rinse with 1×PBS for membrane re-activation. To detect nonspecific binding and cross-reactivity, the membranes were blocked for 1 hour at room temperature and then sequentially incubated with primary antibodies (overnight at 4° C.) and secondary antibodies (for 1 hour at room temperature). Combinations used for testing cross-reactivity and nonspecific binding are listed in FIG. 24B.

Preparation of calibration controls, quality controls and patient samples: Calibration standards and positive quality controls were freshly prepared using normal (non-affected) donor TA lysate (BioIVT). Normal TA standards for a seven-point calibration curve were prepared in incremental amounts of total protein. The high positive control (HPC) was prepared using normal TA lysates mixed with lysis buffer and sample loading buffer to a load of 10 μg per gel. This mixture was heat denatured, divided into single-use aliquots, and frozen between −90° C. and −65° C. for use in validation and sample analyses. The low positive control (LPC) was prepared similarly at a load of 5 ug. The negative controls (NC) were prepared from a commercially available recombinant human DG (rhDG; R & D systems, Minneapolis, MN) expressed in NS0 cells at a concentration of 10 ng in PBS. LGMD2I/R9 lysate samples were diluted in matrix buffer (the same lysis buffer) to achieve an appropriate total protein concentration of 50 μg for L276I homozygous samples and 70 μg for other FKRP genotype samples. These samples were prepared for loading by spiking with 0.001% Bromophenol blue in a final volume of 15 μL.

SDS-PAGE and Western Blot analysis: All samples were boiled and loaded onto a 4-20% Tris-Glycine polyacrylamide gel (Novex, Invitrogen). Proteins were separated and then transferred onto a nitrocellulose membrane (iBlot2, Invitrogen) using the gel transfer device, iblot2 (Invitrogen, Carlsbad, CA, USA). Nonspecific binding sites on the membrane were blocked with Intercept blocking buffer (LI-COR, Lincoln, Nebraska) prior to incubation with a mixture of target-specific primary antibodies. These included the anti-α-DG antibody clone IIH6C4 at 1:1000 dilution, which is specific for glycosylated αDG, and human αDG antibody AF6868 at 0.12 g/mL, specific to αDG regardless of its glycosylated status. The primary antibodies were incubated overnight on a rocking platform at 4° C. Following a series of washes with 1×TBST (tris buffered saline with tween) to remove unbound primary antibodies, the membranes were incubated with a mixture of secondary antibodies. Each blot was incubated with the secondary antibodies at a 1:5000 dilution in 25 mL antibody diluent at room temperature. Membranes were then incubated with a mixture of appropriate near-infrared dye 680RD (700 nm channel) and Alexa Fluor 790 (800 nm channel) secondary antibodies. The membranes were washed again to remove unbound secondary antibodies and images were captured using an Odyssey imager (LI-COR, Lincoln, Nebraska).

Region of Interest (ROI): Densitometric measurements are taken within an ROI to quantify the amount of protein defined by the molecular weight marker (Butler et al. 2019). For lanes with calibration curve samples, an ROI corresponding to 125 to 260 kDa is used to capture signals of fully glycosylated αDG protein of 157 kDa. For non-calibration curve samples, which includes patient samples and HPC, a broader ROI is used from 70 to 260 kDa. This ROI was chosen as patient samples are expected to have varying degrees of αDG glycosylation, resulting from the defective FKRP, and thus are expected to migrate in this molecular weight range. Lower MW, less glycosylated forms of αDG, are gated using the DG antibody AF6868. Table 4 summarizes the ROIs for glycosylated αDG and core αDG signals which are detected simultaneously on separate fluorescence channels (700 nm and 800 nm, respectively) and analyzed using Image Studio™ software by examining boxed regions of the membranes with the guidance of the molecular weight ladder (Chameleon Duo, Licor).

TABLE 4
Regions of Interest Used for Data Collection

| | Molecular Weight | Lowest Anchor |
|---|---|---|
| | Reference Range, kDa | Point, kDa |
| Standard curve samples | 125-260 | 125 |
| QC control (HPC) | 70-260 | 70 |
| Patient Samples | 70-260 | 70 |

Background Correction: For each ROI box drawn, sample signal is corrected for background using Image Studio software according to the following equation: Corrected Signal=Total Signal−(Background×Area of ROI), where background is the sum of pixel intensities of the border selected.

Determination of αDG levels: For each blot, two calibration curves are generated for the 700 nm and 800 nm channels, which correspond to glycosylated and core αDG, respectively. These curves are created using a 7-point dilution series ranging from 1.25-25 μg of lysate from the selected normal donor, TA1. Raw fluorescence signals from the 700 nm and 800 nm channels for each patient's biopsy lysate are interpolated to their corresponding calibration curve. This allows for determination of normal TA equivalent levels of glycosylated and core αDG, respectively, in μg. The resulting mean μg value for each sample (N=2) is then normalized by the total protein loaded, which is used to calculate the percent of normal glycosylation (Suzuki et al. 2011).

Data Normalization and percentage calculation: As described above, levels of glycosylated αDG and core αDG are determined by interpolation against a calibration curve of normal control TA. To compute the percentage of core and glycosylated αDG, the mean interpolated μg value for each sample is further normalized by the total biopsy lysate protein concentration.

$$\% Normal α DG = \frac{Interpolated value ( μ g )}{Protein loaded ( μ g )} * 100$$

### Results

Establishment of a Suitable Assay System: Therapeutic effects on dystrophin expression in muscular dystrophies like Duchenne muscular dystrophy have been directly examined in skeletal muscle in various clinical trials (Alfano et al. 2019; Charleston et al. 2018; Barthelemy et al. 2020). For conditions with distal limb symptoms, such as LGMD2I/R9, the tibialis anterior (TA) is the preferred muscle biopsy site due to its histological and functional susceptibility to the disease (Joyce et al. 2012). The TA is a practical choice for biopsy as it has identifiable physical landmarks and can be easily accessed with a fine core needle (Barthelemy et al. 2020), reducing patient discomfort. The application of the FNA method for TA extraction, which results in the limitation in tissue quantity, necessitates a customized assay methodology for the detection of αDG levels (Iachettini et al. 2015). A sensitive and reliable approach is necessary, particularly when working with patient sample biopsies of LGMD2I/R9, which exhibit severely reduced, broad range of glycosylated states of αDG.

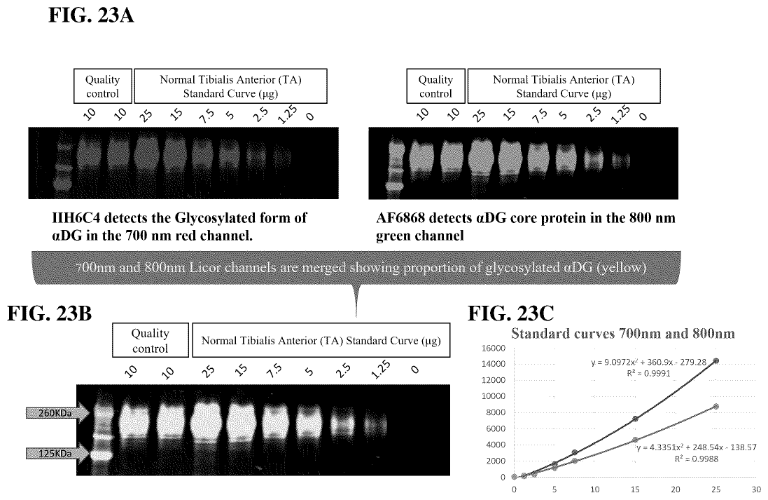

Classical Western blotting (WB) can yield variable results due to its non-quantitative ability, only indicating protein presence but not quantity. Other potential issues include off-target antibody binding and the limitation of detecting only specific proteins. Moreover, complications are usually seen with ECL detection, such as bands obscured in overexposed areas. Keeping this in mind, an innovative, multiplexed, fluorescence-based WB method was developed that analyzes the αDG glycosylation state by simultaneously measuring core and glycosylated αDG in TA biopsy samples from patients with LGMD2I/R9. The protein extraction method from muscle biopsies uses an SDS-urea buffer for improved homogenization, which resulted in increased total protein yield and sharper protein bands. The higher efficacy of SDS-urea buffer compared to non-denaturing, nonionic detergent for extracting muscle biopsy for αDG determination was demonstrated previously (Peach et al. 2015). The selection of IIH6C4 and AF6868 antibodies was substantiated by multiple investigators (Briggs et al. 2016; Alhamidi et al. 2017; Lee et al. 2019; Cataldi et al. 2018; Wu et al. 2022; Willis et al. 2022). It was determined that AF6868 recognizes both the α and β subunits of rhDG and was therefore selected for use in the WB assay. The assay uses a more quantitative and reliable approach by fluorescence-based detection method which has the advantage of capturing a broader dynamic range with improved reproducibility in contrast to the enzyme based chemiluminescence detection technique. Additionally, fluorescence-based dyes enable multiplexing by allowing simultaneous detection of multiple proteins, based on the different excitation and emission wavelength of the dyes, thus emitting light of distinct frequencies that can be individually analyzed. A calibration curve derived from normal TA was used to determine the relative normal amounts of αDG in the patient samples and this interpolation method effectively corrects for inter-assay variability associated with antibody performance and the transfer process from gel to blot. To ensure the reliability of the results, quality control measures were implemented to monitor both inter-assay and intra-assay performance (FIGS. 23A-23C).

Evaluating antibody specificity in a multiplexed system: The detection of each form of αDG requires exposure to matching pairs of primary and secondary antibodies. Because of the excess of other proteins, in particular albumin and fragments of immune globulin, in the muscle extracts, inappropriate recognition by the secondary antibodies in western blot analysis can potentially lead to increased background signal (Miyara et al. 2016). To evaluate this background (cross-reactive) reaction, varying amounts of muscle lysate, 5, 15, and 30 μg, were pipetted dropwise onto a membrane and exposed to multiple combinations of secondary and primary antibodies. The linear increase of signal intensity with the amounts of muscle lysate applied to the membrane indicates that the ratio of primary and secondary antibodies is appropriate for the amount of protein applied. Further, the specificity of the secondary antibodies to primary antibodies is demonstrated by the absence of signal when non-matching antibodies are mixed at varying lysate concentrations. The linear increase of signal intensity with the amounts of muscle lysate applied to the membrane indicates that the signals are proportional to the total protein loaded. For example, the secondary antibody for the 700 nm channel is specific for mouse IgM, which is the isotype of the IIH6C4 primary antibody. When this secondary antibody was mixed with the AF6868 primary antibody, which was a sheep IgG, no signal at 700 nm was observed because there was no binding between the incompatible primary and secondary antibodies. Similarly, the secondary antibody for the 800 nm channel is specific for sheep IgG, which is the isotype of the AF6868 primary antibody. If this secondary antibody was mixed with the IIH6C4 primary antibody, which was a mouse IgM, no signal at 800 nm was reported, confirming no binding between the secondary and primary antibodies. When these antibodies are present in the right combination, they give a positive signal, proportional to the total protein loaded (FIGS. 24A-24D).

Validating Antibodies Using Negative Controls: The assay specificity was further assessed with lysates prepared from a commercially available DAG1 knockout human embryonic kidney (HEK) 293T cell line (lacking dystroglycan) and a wild-type HEK293T control cell line analyzed at g total protein load for the presence of core αDG and glycosylated αDG. Overall, reduced signal intensity for both channels (700 nm for glycosylated and 800 nm for core αDG) was observed in the DAG1 knockout cell lysate compared with the wild-type cell line (Table 5).

TABLE 5
Raw signal intensities for DAG1 KO and WT HEK293T

| | | 700 nm Channel | 800 nm Channel |
|---|---|---|---|
| Sample | N | RFU (IIH6C4) | RFU (AF6868) |
| Hu DAG1 KO HEK293T | 1 | 385 | 467 |
| (20 μg protein) | | | |
| Hu WT HEK293T | 1 | 1,390 | 1,960 |
| (20 μg protein) | | | |

Abbreviations: KO = knockout; RFU = relative fluorescence units; WT = wild type; Hu = human.

Separately, a negative validation control for the IIH6 antibody using human recombinant αDG expressed in myeloma cells (with a reduced molecular weight of 70 kDa as expected for hypo glycosylated protein), showed significantly reduced 700 nm:800 nm signal ratios, with the core αDG relatively higher (˜6 fold) intensity than glycosylated αDG intensity further confirming the suitability of both IIH6C4 and AF6868 antibodies (Table 6).

TABLE 6
Raw signal intensities for recombinant αDG protein

| | | 700 nm Channel RFU | | | | 800 nm Channel RFU | | | |
|---|---|---|---|---|---|---|---|---|---|
| | | (IIH6C4) | | | | (AF6868) | | | |
| Sample | N | Mean | SD | Median | IQR | Mean | SD | Median | IQR |
| Negative | 9* | 10,483 | 2,426 | 11,200 | 3,680 | 63,300 | 6,465 | 64,100 | 5,900 |
| validation | | | | | | | | | |
| control | | | | | | | | | |
| (100 ng protein) | | | | | | | | | |

Abbreviations: KO = knockout; IQR = interquartile range; SD = standard deviation; RFU = relative fluorescence units; WT = wild type.
*Experiment was done by 2 analysts over a period of 3 months

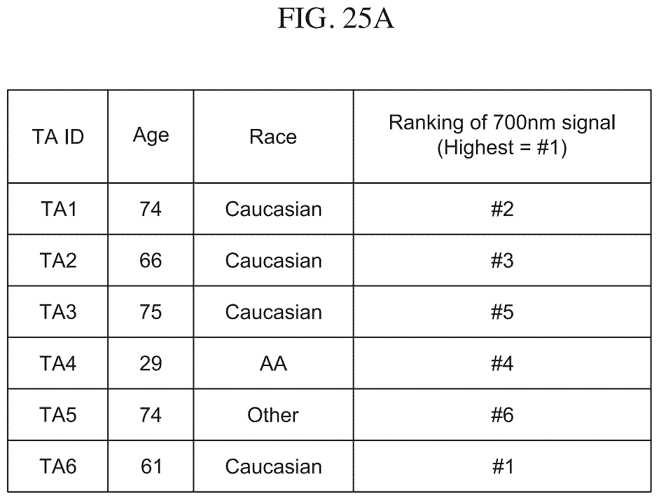

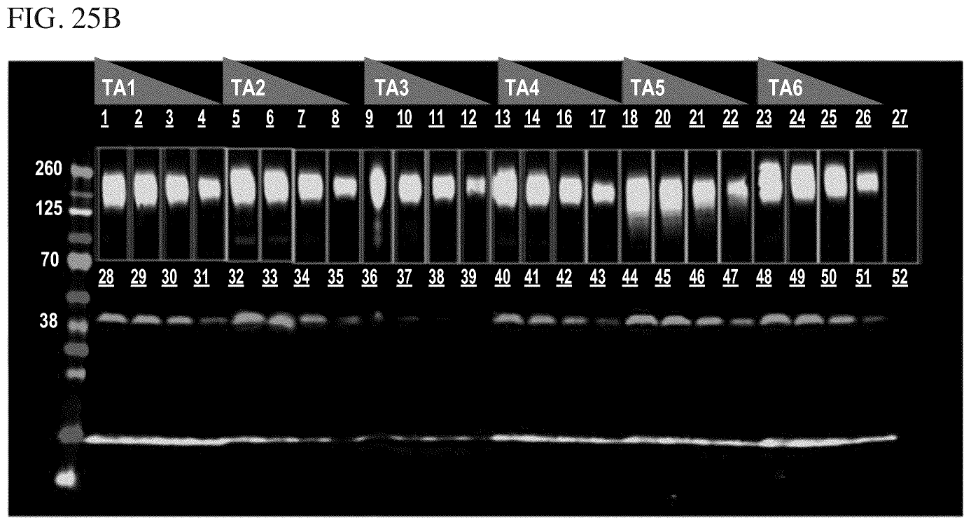

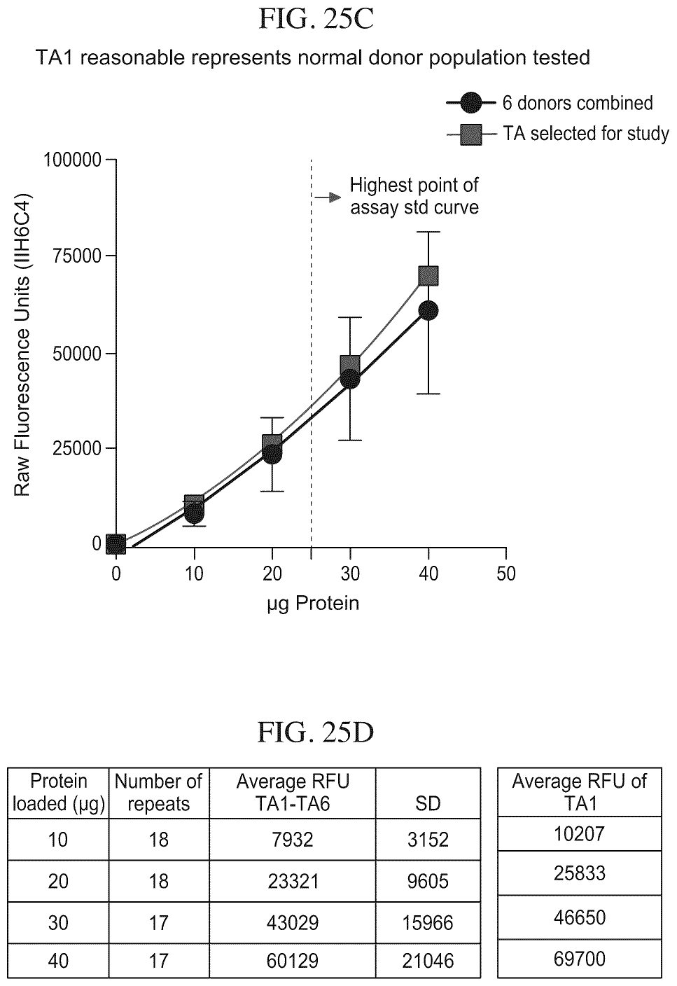

Evaluation of inter-subject variability of αDG in TA samples for Calibration and Quality Control: Analysis of TA muscle lysates from six non affected normal donors (FIGS. 25A-25D) was conducted to explore the variability of banding patterns and the levels of αDG expression. The data demonstrated that glycosylation did not appear to be age-dependent in the six samples evaluated, which ranged from 29 to 75 years. The banding pattern was consistent among all six donors, with αDG protein bands observed at the ˜160 kDa molecular weight range, as anticipated for fully glycosylated protein. The average signal intensity of TA1 was comparable to the average signal intensity of all six donor TA samples across three blots and at four different concentrations. It was important to identify a balance between tissue availability and performance. The overlap in signal intensity and the sufficient availability of TA1 enabled its use as control in multiple assays across different studies, thereby minimizing inter-assay and inter-study variability.

Assay validation: This study included validation of a WB blot assay for the simultaneous detection of glycosylated αDG containing matriglycan, the functional form of αDG, and core αDG in lysates from muscle biopsies. The small abundance of dystroglycan in muscle biopsies from patients required optimization of protein solubilization (Peach et al. 2015) for an improved protein yield and, therefore, SDS-urea buffer was chosen as it satisfied these requirements. Previous lysis condition which utilized a commercial RIPA buffer was found to be inefficient in extracting membrane bound proteins. Notably, RIPA is a commonly utilized lysis buffer for tissue samples. However, the extremely low abundance of the target protein αDG in certain patient samples necessitates using SDS-urea to improve yield of extracted protein from muscle tissue. As described previously, comparison of extraction methods for the detection of the fluorescent signals from selective antibodies were collected by a two channel LI-COR WB imager. The assay was calibrated using a calibration curve, in which various amounts of protein lysate from normal muscle was diluted into lysis buffer. Validation of the assay was performed using the key parameters proposed in the Bioanalytical Method Validation Guidance for Industry from the FDA (FDA guidance) which included: (1) assay precision, which is the degree of agreement among repeated measurements of the same sample under the same conditions; (2) accuracy, which is the closeness of agreement between the measured values and the true values of the analytes; (3) sensitivity, which is the ability of the assay to detect low levels of the analyte; (4) linearity which is the ability of the assay to produce results that are directly proportional to the concentration of the analyte within a given range; (5) range which is the interval between the upper and lower levels of the analyte that can be measured by the assay with acceptable accuracy and precision; and (6) robustness, which is the ability of the assay to maintain its performance under minor variations in environmental or operational conditions. Every assay gel incorporated a calibration curve, negative control (for IIH6C4 antibody) and positive controls prepared from the selected normal donor TA tissue. The fluorescence signal intensities for each tissue lysate and Quality Controls (QCs) were converted to masses using the calibration curve. This allowed for the interpolation of the relative normal levels of αDG and then normalization to total protein loaded for estimation of % normal levels.

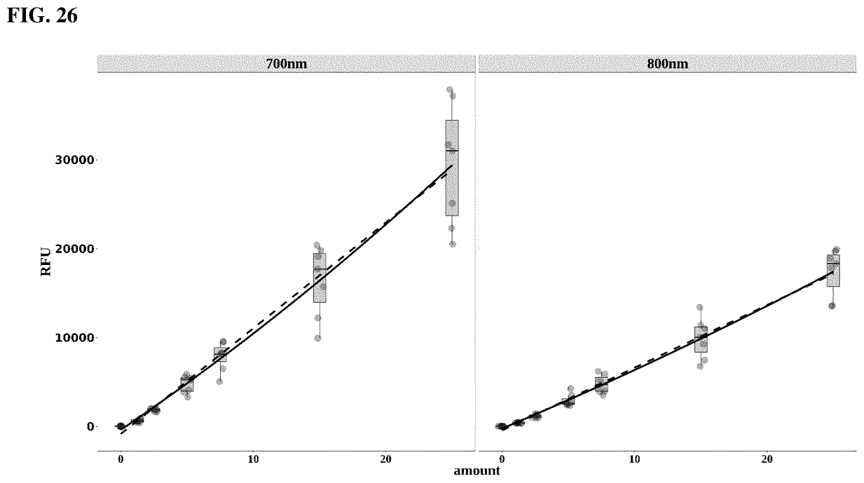

Optimal model for the calibration curves: In the absence of purified core and glycosylated αDG, a calibrant was generated from a serial dilution between 0-25 μg normal TA1 lysate. The signal to concentration response was established by regression to a linear or quadratic model. Since small variations in the procedure significantly affect the electrophoretic transfer and retention of proteins on the blotting membrane, a calibration curve was required on each blot. This is evident from the spread in values in FIG. 26 and the variation in the parameters for the regression curves. The quadratic model accommodates the data better than a linear fit, consistent with the lower AIC (a measure for information not explained by the model) value and in most cases with the calculated p<0.05 for the quadratic term (Table 7). Furthermore, since the p-values for the intercepts obtained from regression are not significant in most cases and the confidence intervals cross zero, it could be set to the physically sensible value zero. However, “nonproportional” concentration response curves with intercepts different from zero have been reported by other authors (Butler et al. 2019; Pitre et al. 2007) for WB. Interestingly, previous reports suggest WB calibration curves fit to polynomial or exponential terms (Pitre et al. 2007). For quantitative evaluation of the blots, the relative amounts in μg of core and glycosylated αDG, in terms of total protein in the samples, are determined by interpolation using the inverted calibration curve of the signals measured with the protein standard mixture (Heidebrecht et al. 2009; Suzuki et al. 2011). This relative amount is then divided by the relative amount of a normal sample to express the relative level of glycosylated αDG or core DG in a patient sample.

TABLE 7
Details of linear and quadratic fitting of calibration curves.

| | Slope | (Slope | | (Intercept | | Detection | Decision | Adjusted |
|---|---|---|---|---|---|---|---|---|
| | (RFU/ug) | CI) | Intercept | CI) | AIC | limit (ug) | limit (RFU) | R² |
| Run_2 | 1,298** | (1,211, | −627 | (−1,632, | 120 | 3.4 | 3742 | 0.99 |
| | | 1,385) | | 378) | | | | |
| Run_4a | 806** | (728, | −663 | (−1,574, | 119 | 4.9 | 3274 | 0.99 |
| | | 885) | | 247) | | | | |
| Run_4b | 888** | (847, | −353 | (−834, | 110 | 2.4 | 1746 | 1 |
| | | 930) | | 128) | | | | |
| Run_5 | 1,295** | (1,219, | −1,260* | (−2,142, | 118 | 3 | 2580 | 0.99 |
| | | 1,371) | | 378) | | | | |
| Run_6 | 1,490** | (1,362, | −1,581 | (−3,067, | 125 | 4.3 | 4859 | 0.99 |
| | | 1,619) | | −95) | | | | |
| Run_10 | 1,523** | (1,415, | −1,435 | (−2,693, | 123 | 3.6 | 4027 | 0.99 |
| | | 1,632) | | −178) | | | | |
| Run_11 | 1,026** | (968, | −93 | (−771, | 114 | 2.9 | 2858 | 0.99 |
| | | 1,085) | | 584 | | | | |
| Run_2 | 836** | (773, | −332 | (−1,062, | 115 | 3.8 | 2833 | 0.99 |
| | | 899) | | 397) | | | | |
| Run_4a | 534** | (488, | −337 | (−872, | 111 | 4.3 | 1981 | 0.99 |
| | | 580) | | 198) | | | | |
| Run_4b | 536** | (513, | −161 | (−431, | 102 | 2.2 | 1022 | 1 |
| | | 560) | | 110) | | | | |
| Run_5 | 778** | (736, | −686* | (−1,169, | 110 | 2.7 | 1420 | 1 |
| | | 820) | | −203) | | | | |
| Run_6 | 715** | (656, | −744 | (−1,419, | 114 | 4.1 | 2178 | 0.99 |
| | | 773) | | −70) | | | | |
| Run_10 | 790** | (723, | −842 | (−1,617, | 116 | 4.3 | 2517 | 0.99 |
| | | 857) | | −67) | | | | |
| Run_11 | 746** | (706, | −55 | (−525, | 109 | 2.7 | 1994 | 1 |
| | | 787) | | 415) | | | | |

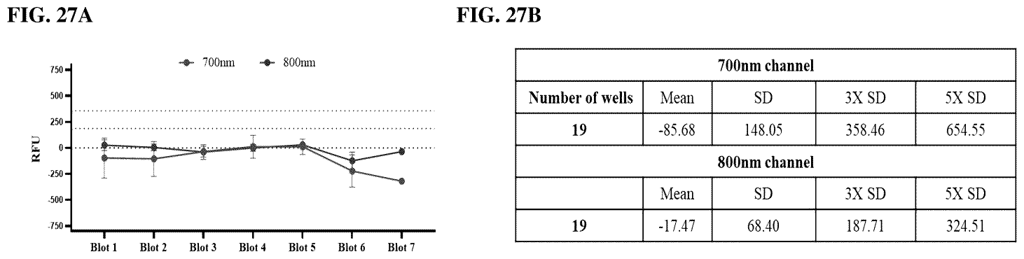

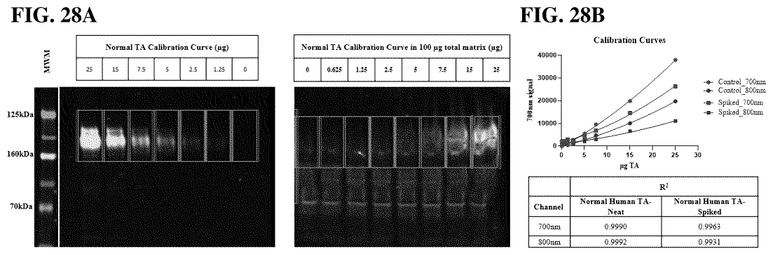

Background signal and limit of detection (LOD): The stability of the blank signal in the assay is illustrated in a Levi-Jennings plot (FIG. 27A) of the measured signals versus the gel number. The samples consist of protein-free buffer which are analyzed in an immunoblot along with the samples used for calibration of the assay. The mean and standard deviation of all background wells across all empty lanes was calculated and are shown in FIG. 27B. The LOD was derived using the noise level above the blank by determining the signal of 3 times the standard deviation. The results of the estimation and the inverse predicted values are summarized in FIGS. 28A-28B.

Evaluating assay sensitivity and lower limit of quantitation: An initial assessment of LGMD2I/R9 patient samples indicated that a high amount of protein is necessary to obtain a quantifiable signal of αDG. This necessitated an evaluation of the assay's sensitivity at a matrix concentration of 100 μg. In the absence of a suitable matrix deficient in glycosylated αDG, TA lysate from a transgenic mouse with an FKRP-P448L mutation, which results in severely hypo glycosylated αDG, which runs at a molecular weight lower than that of normal human control, was used to assess assay sensitivity. In a matrix containing 100 μg total protein from both mouse and human TA, varying amounts of normal control TA lysate was added to evaluate the sensitivity range. As shown in FIGS. 28A-28B, a consistent increase in signal was observed across the high matrix TA calibration curve. The difference in slope is attributed to possible interference from the inherent αDG glycosylation signal from the mouse matrix tissue. Further, the consistency of glycosylated αDG/core αDG signal ratio was also reflected in a coefficient of variation (CV) of less than 30% and an overall R² of >0.99 from a quadratic fit. In the previously described HEK model, the signal for glycosylated αDG at 700 nm was found to be lower than that for the core protein at 800 nm for the wildtype. This contrasts with the trend in the calibration curves, where the 700 nm signal is higher than the signal at 800 nm at the same protein load. It is worth noting that HEK cells produce the critical mucin domain, which is required for matriglycan formation, very inefficiently. Therefore, signal from this cell system can be assumed to differentiate between the presence and absence of the critical epitope (Sun et al. 2022; Harrison et al. 2012). Further analysis of the calibration located the detection limit at roughly 1p g.

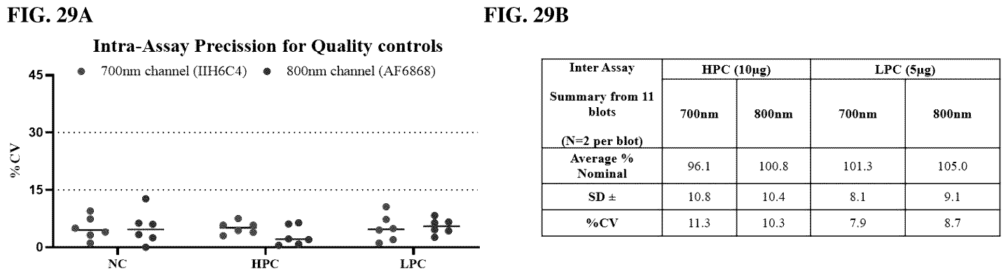

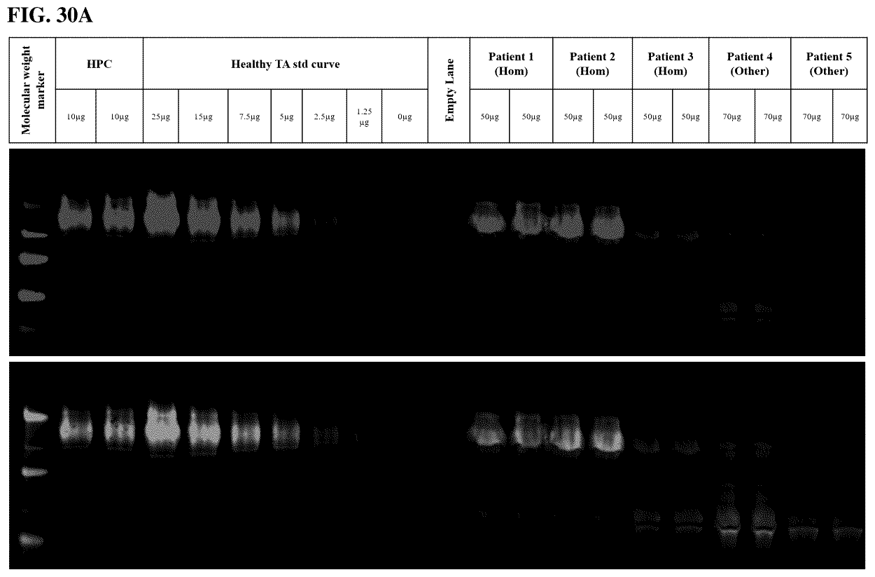

Evaluating inter and intra assay precision: Inter- and intra-assay precision was evaluated by collecting the raw fluorescence signal intensities for test and control samples. To examine intra-assay operator precision, two analysts ran each of the validation controls in duplicate and on six gels on different days. For assay precision determination, each of two analysts ran TA lysates at 10 μg and 5 μg per lane in quadruplicate on a gel for a total of six gels across multiple days. Assay performance is monitored using quality controls from the same donor. Intra-assay precision is evaluated using a negative control of purified protein and positive controls of TA lysate. Variability in signal between sample replicates is assessed. For inter-assay precision, interpolated (back-calculated) values of the positive controls were tracked. Results demonstrate good assay reproducibility for both 700 nm and 800 nm channels with a coefficient of variation below 15%. (See FIGS. 29A-29B).

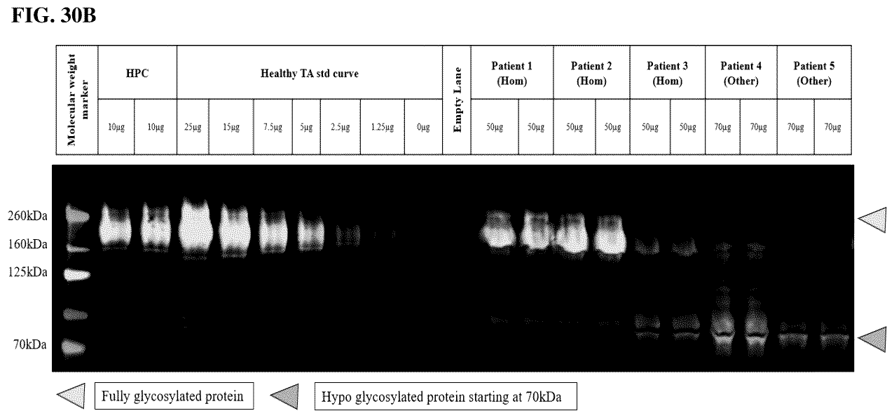

Patient sample analysis, banding patterns, and gating differences: The validated assay was used to evaluate levels of glycosylated and core αDG in patient biopsy samples. A calibration curve was generated with normal human TA samples to allow simultaneous quantification of glycosylated and core αDG levels, and samples were run in duplicate on each blot, including the positive control to assess assay performance. The Region of Interest (ROI) is a standardized area on the blot, specifically chosen for the detection and analysis of the protein under study. This region is selected based on the target protein's molecular weight and the separation achieved during gel electrophoresis. The ROI plays an important role in the precise quantification and analysis of WB. It determines data accuracy and reproducibility and facilitates the comparison of protein expression across samples tested in varying quantities (Taylor et al. 2013; Bass et al. 2017; Aldridge et al. 2008). The blot shown in FIGS. 30A-30B includes three patients with homozygous L276I/L276I genotypes (Patients 1-3) and two patients with other FKRP genotypes, participating in the natural history study (MLB-01-001). The banding patterns observed for the L276I/L276I patients 1 and 2 resemble the normal controls, but that of patient 3 exhibits a greater heterogeneity of glycosylation patterns. It is interesting to note that L276I/L276I Patient 3 exhibits a broader range of hypo glycosylated αDG bands, with molecular weights between 70-160 kDa, compared to patients 4 and 5 who have other FKRP genotypes, suggesting the presence of a broad spectrum of glycosylation pattern in LGMD2I/R9 patients compared to normal donors.

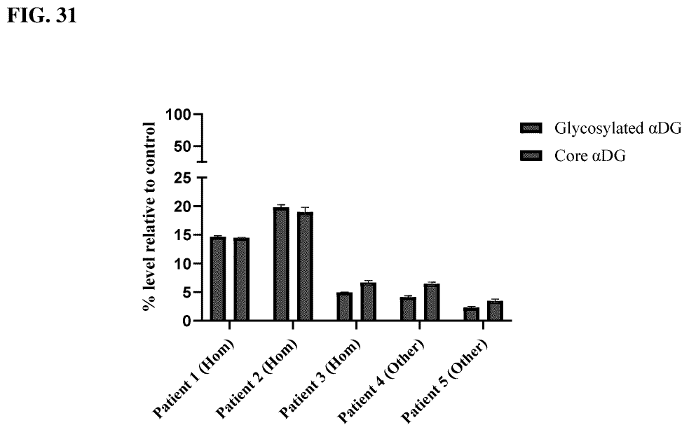

FIG. 31 shows relative levels of αDG in skeletal biopsies of patients with LGMD2I/R9. The figure presents relative levels of αDG from patient biopsies in FIGS. 30A-30B. The relative percent levels of αDG were determined from the 700 nm signal (for glycosylated αDG) and 800 nm signal (for core αDG) were interpolated to the calibration curve. The resulting relative μg amount was normalized by the total protein loaded.

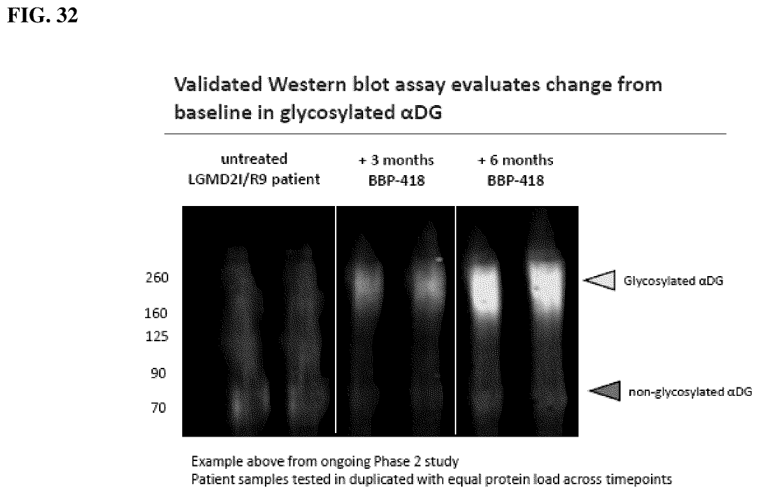

FIG. 32 shows that the validated Western blot assay effectively evaluates change from baseline in glycosylated αDG as early as 3 months after commencement of treatment with ribitol (BBP-418).

### Discussion

The present disclosure describes the development and application of a multiplexed fluorescence-based WB method for the quantitative determination of glycosylated and core αDG in skeletal muscle biopsies. For validation samples from normal donors and patients with LGMD2I/R9, a disease characterized by chronic hypo-glycosylation of αDG, were compared. LGMD2I/R9 patients have mutations in the fukutin-related protein (FKRP). Partial loss of function in the FKRP enzyme leads to incompletely glycosylated (“hypo-glycosylated”) αDG, which then fails in its critical role of stabilizing muscle contraction and maintaining muscle cell membrane integrity (Georganopoulou et al. 2021; Endo 2015). The developed assay can distinguish between reduced glycosylation levels of αDG from compound heterozygous LGMD2I/R9 patients expressing both L276I and another defective variant of FKRP. Since the readout of the assay is directly linked to the molecular defect, it may be considered a marker for the severity of disease and potentially be used to interrogate effects of therapeutic intervention (Godfrey et al. 2007).

Several studies have used IHC for the detection of glycosylated and core αDG in samples of muscle tissues biopsies (Fritschy 2008; Johnson et al. 2018); comparative evaluation of protein expression is difficult due to variability of protein staining, lack of reusable calibration controls and limited detection range (Fritschy 2008). Similarly, the analysis of muscle lysate by MS techniques is not suitable for quantitation of core and glycosylated protein as they require complex pre-purification steps and analysis of glycosylation states of αDG protein is difficult to perform (Chandel and Campbell 2023; Harrison et al. 2012). Enzyme-linked immunosorbent assays are another tool for the quantitation of biomarkers, but available commercial assays do not differentiate between various glycosylated forms of αDG, and tissue lysis buffer interferes with ELISA detection (Crowe et al. 2016). Thus, Western blotting appears to be most appropriate, as it can be adapted for quantitative analysis of complex protein mixtures and the ability to track specific molecular weight(s) provide potentially significant information for diagnosis of hypo-glycosylation. In addition, combined with fluorescence detection WBs permit multiplex detection (Anderson and Davidson 1999, Schutz-Geschwender et al. 2004). The use of a Western blotting assay as described herein, rather than another assay such as IHC or ELISAPP, is innovative and enables measurement of αDG to be useful as a biomarker in evaluating subjects undergoing treatment for dystroglycanopathies in clinical studies and in conventional treatment settings.

The primary antibodies, IIH6C4 and AF6868, and their utility in muscle biopsy assessment have been studied for the detection of glycosylated and total αDG, respectively (Michele et al. 2002; Muntoni et al. 2011). The monoclonal antibody IIH6C4 has been used to monitor the increase of matriglycan content in αDG in FKRP deficient mice upon treatment with ribitol over time (Cataldi et al. 2018) and its reduction due to glycosidase treatment in vitro (Chandel and Campbell 2023). An alternate antibody, VIA4-1, has been used for the detection of glycosylated αDG in WB of tissue extracts from LGMD patients. In contrast to VIA4-1, IIH6C4 can prevent the binding of glycosylated αDG to laminin and may be considered a functional surrogate of the native ligand binding. Accordingly, as the impaired ability of αDG to interact with laminin is thought to be the cause of LGMD, the signal resulting from IIH6C4 binding may have a correlation to disease severity. Thus, the pairing of IIH6C4 and AF6868 appears to be the appropriate choice to monitor the levels of glycosylated (and therefore functional) versus total αDG in patients.

As the assay is developed for the analysis of fine needle biopsies, the small sample size required effective and optimal tissue lysis prior to separation on gels. Efficient recovery of the proteins is particularly important for patient samples due to the low abundance of total and specifically glycosylated αDG. In the present study, samples from tibialis anterior were processed for evaluation. TA is one of the most active lower leg muscles and is considered optimal for biomarker studies in other muscular diseases (Carberry et al. 2012; Joyce et al. 2012; Iachettini et al. 2015). From a practical perspective of obtaining tissue biopsy samples from patients, TA has consistent physical landmarks and is relatively easy to biopsy using a small core needle, thereby limiting patient burden. During WB development, comparison of different buffer systems for extraction of proteins from TA confirmed the higher efficiency of denaturing mixtures composed of SDS/urea and a reducing agent (Zardini et al. 1993) relative to commercial buffers containing nonionic detergents. In the context of the present study with dystroglycan, the ability of the buffer system to dissociate and solubilize large protein complexes with laminin and myosin is particularly advantageous (Anderson and Davison 1999; Miskiewicz and MacPhee 2019). Low abundance of dystroglycan protein requires large volumes of sample to be separated by the gel. Under these assay conditions, in contrast to buffers with SDS, a concomitant excess of nonionic detergents disrupts the protein migration.

Proteins on WB may be detected by the ECL or by fluorescence intensity. In contrast to fluorescence signals, ECL detection is sensitive to small variations in the execution of the assay, has a narrow dynamic range and often does not produce a linear response proportional to the target antigen. ECL chemistry at the basis of the reaction depends on timing and substrate availability (Pillai-Kastoori et al. 2020). Early reports on the abundance of αDG between healthy normal and patient samples varied widely, the extended dynamic range of fluorescence detection compared to that of ECL makes it particularly attractive for comparative studies related to LGMD. Moreover, because of the reduced variability relative to ECL, fluorescence detection appears to be more suitable for serial experiments on patient samples. The reported WB assay is based on the detection of glycosylated relative to total αDG on the same blot. This allows for the possibility of simultaneous detection rather than a need for error-prone stripping of blotting membranes required for ECL. The use of fluorescence detection to quantitate a muscle protein of clinical interest has also been reported for dystrophin, a protein implicated in a disease similar to LGMD (Schnell et al. 2019).

With only 0.0013% (89 nmol/1 g mouse skeletal muscle extract) dystroglycan expected in tissue extracts (Johnson et al. 2013), matrix effects and cross reactivity may pose a potential problem. Notably, even with SDS-urea yielding a higher amount of protein than a commercial RIPA buffer, 0.0013% really tests the limit of detection. Accordingly, a beneficial improvement of the Western blotting assay described herein lies in having a lower limit of quantifiable detection not previously noted in peer reviewed publications. The selectivity of both secondary antibodies was demonstrated by the absence of signals, when mismatched pairs of primary and secondary antibodies were tested in a multiplexed dot-blot assay. The selectivity of the primary antibodies for their specific epitopes was inferred by a five-fold difference in signal between HEK cell extracts from dystroglycan knock-out and wild type mice.

A protein standard for glycosylated αDG is not available as the expressed protein with complete mucin modification reactive with the antibody could not be obtained from a commercial source. Thus, extracts of healthy control muscle were used to generate a calibration curve and the amount of glycosylated protein was expressed in terms of microgram of the total healthy extract. Similar quantitation of a mixture of components as relative amounts using “mixed calibration curves” have been used in HPLC analysis of plasma samples and metabolomics (Chen et al. 2017; Liang et al. 2010). As all experiments presented in this Example use the same healthy lysate, the reported amounts albeit relative, are consistent among each other. Since the calibration curve is generated on each gel, fluctuations in signal intensity of αDG are normalized between individual WB and quantitative comparison is possible between gels. Comparison with the calibration curve also assures that both forms of αDG are applied to the gel in a range with signals increasing monotonically with dose, as generally recommended for quantitative WB. Although calibration curves for WB are usually presumed to be linear, statistical analysis of the presented data yielded a better fit for a parabolic curve. This is consistent with some reports in literature (Butler et al. 2019). The LLOQ extrapolated from this curve equals 1p g total protein, which translates into an estimated detection limit of 13 μg of dystroglycan in the muscle extract. Although very small, this estimate appears consistent with detection limits for fluorescent WB reported in the literature. The low detection limit compares favorably with an ECL based WB assay reported for dystrophin, which was assessed around 3 ng. Similar to ECL based assay for dystrophin, the αDG assay described herein has a precision expressed as a coefficient of variation of 30%. With its high sensitivity this WB dystroglycan assay is particularly valuable for analysis of patient samples, as they are characterized by low expression/low levels of glycosylated and total dystroglycan and a generally lower content of intact muscle fibers.

The results described herein of testing patient samples from compound heterozygous LGMD2I patients expressing both L276I and another defective FKRP variant indicated reduced levels of glycosylated αDG relative to their homozygous counterparts. Though a correlation between severity of disease and level of αDG glycosylation has been suggested (Mercuri et al. 2003; Brown et al. 2004), such a correlation has not been widely accepted and has been disputed (Jimenez-Mallebrera et al. 2009; Alhamidi et al. 2017). The discrepancies may have resulted from limitations in the quantitative interpretation due to the ubiquitous use of ECL detection and the lack of calibration, normalization procedures and differences in the muscle biopsy location. It is unclear if the necessary correction of differences in the ECL reaction between blots could be carried out under conditions of the reported methods. In contrast, the approach described herein uses a more stable detection system and enables reliable comparison, as all results are normalized to a calibration curve on each blot. Thus, consistent adaptation of the methodology of this report for evaluating glycosylated αDG and data normalization may pave the way to further understanding of hypo-glycosylation and its relation to disease severity.

ABBREVIATIONS

| Abbreviation | Definition |
|---|---|
| αDG | alpha-dystroglycan |
| βDG | beta-dystroglycan |
| AE | Adverse event |
| AF6868 | Sheep Anti-Human Dystroglycan Antigen |
| | Polyclonal Antibody |
| BID | twice daily |
| BLQ | below limit of quantitation |
| C57 | C57BL/6 is a common inbred strain of laboratory |
| | mouse used as “genetic background” for |
| | genetically modified mice for use as models |
| | of human disease |
| CDP-ribitol | Cytidine 5-diphosphotase ribitol |
| CI | Confidence interval |
| CK | creatine kinase |
| CMD | Congenital muscular dystrophy |
| DAG1 | dystroglycan 1 |
| DGC | dystrophin-associated glycoprotein complex |
| FKRP | Fukutin-related protein |
| FKTN | Fukutin |
| FVC | forced vital capacity |
| g | gram |
| GMR | Geometric mean ratio |
| IIH6C4 | monoclonal anti-α-dystroglycan antibody |
| ISPD | isoprenoid synthase domain containing protein |
| kDa | kilodaltons |
| kg | kilogram |
| LGMD | limb girdle muscular dystrophy |
| LGMD2i or | limb girdle muscular dystrophy type 2i |
| LGMD2I/R9 | |
| μg | microgram |
| ng | nanogram |
| nm | nanometer |
| NSAD | North Star Assessment for Limb Girdle |
| | Type Muscular Dystrophies |
| pg | picogram |
| PUL2.0 | Performance of upper limb test 2.0 |
| QD | once daily |
| rib | ribitol |
| Ribitol 5P | ribitol-5-phosphate |
| TA | Tibialis anterior |
| TMEM | transmembrane protein 5 |
| WB | Western blot |
| 10 MWT | 10-meter walk test |
| 100 MTT | 100-meter timed test |

### REFERENCES

Aldridge G M, Podrebarac D M, Greenough W T, Weiler I J (2008) The use of total protein stains as loading controls: an alternative to high-abundance single-protein controls in semi-quantitative immunoblotting. J Neurosci Methods 172: 250-4. doi.org/10.1016/j.jneumeth.2008.05.003
Alfano L N, Charleston J S, Connolly A M, Cripe L, Donoghue C, Dracker R, Dworzak J, Eliopoulos H, Frank D E, Lewis S, Lucas K, Lynch J, Milici A J, Flynt A, Naughton E, Rodino-Klapac L R, Sahenk Z, Schnell F J, Young G D, Mendell J R, Lowes L P (2019) Long-term treatment with eteplirsen in nonambulatory patients with Duchenne muscular dystrophy. Medicine (Baltimore) 98: e15858. doi.org/10.1097/MD.000000000001585
Alhamidi M, Brox V, Stensland E, Liset M, Lindal S, Nilssen O (2017) Limb girdle muscular dystrophy type 21: No correlation between clinical severity, histopathology and glycosylated alpha-dystroglycan levels in patients homozygous for common FKRP mutation. Neuromuscul Disord 27: 619-26. doi.org/10.1016/j.nmd.2017.02.015
Anderson L V, Davison K (1999) Multiplex Western blotting system for the analysis of muscular dystrophy proteins. Am J Pathol 154: 1017-22. doi.org/10.1016/S002-9440(10)65354-0
Balci-Hayta B, Talim B, Kale G, Dincer P (2018) LARGE expression in different types of muscular dystrophies other than dystroglycanopathy. BMC Neurol 18: 207. doi.org/10.1186/s12883-018-1207-0
Barresi R, Campbell K P (2006) Dystroglycan: from biosynthesis to pathogenesis of human disease. J Cell Sci 119: 199-207. doi.org/10.1242/jcs.02814
Barthelemy F, Woods J D, Nieves-Rodriguez S, Douine E D, Wang R, Wanagat J, Miceli M C, Nelson S F (2020) A well-tolerated core needle muscle biopsy process suitable for children and adults. Muscle Nerve 62: 688-98. doi.org/10.1002/mus.27041
Bass J J, Wilkinson D J, Rankin D, Phillips B E, Szewczyk N J, Smith K, Atherton P J (2017) An overview of technical considerations for Western blotting applications to physiological research. Scand J Med Sci Sports 27: 4-25. doi.org/10.1111/sms.12702
Briggs D C, Yoshida-Moriguchi T, Zheng T, Venzke D, Anderson M E, Strazzulli A, Moracci M, Yu L, Hohenester E, Campbell K P (2016) Structural basis of laminin binding to the LARGE glycans on dystroglycan. Nat Chem Biol 12: 810-4. doi.org/10.1038/nchembio.2146
Brockington M, Yuva Y, Prandini P, Brown S C, Torelli S, Benson M A, Herrmann R, Anderson L V, Bashir R, Burgunder J M, Fallet S, Romero N, Fardeau M, Straub V, Storey G, Pollitt C, Richard I, Sewry C A, Bushby K, Voit T, Blake D J, Muntoni F (2001) Mutations in the fukutin-related protein gene (FKRP) identify limb girdle muscular dystrophy 21 as a milder allelic variant of congenital muscular dystrophy MDC1C. Hum Mol Genet 10: 2851-9. doi.org/10.1093/hmg/2.2851
Brown S C, Torelli S, Brockington M, Yuva Y, Jimenez C, Feng L, Anderson L, Ugo I, Kroger S, Bushby K, Voit T, Sewry C, Muntoni F (2004) Abnormalities in alpha-dystroglycan expression in MDC1C and LGMD2I muscular dystrophies. Am J Pathol 164: 727-37. doi.org/10.1016/s002-9440(10)63160-4
Butler TaJ, Paul J W, Chan E C, Smith R, Tolosa J M (2019) Misleading Westerns: Common Quantification Mistakes in Western Blot Densitometry and Proposed Corrective Measures. Biomed Res Int 2019: 5214821. doi.org/10.1155/2019/5214821
Carberry S, Zweyer M, Swandulla D, Ohlendieck K (2012) Profiling of age-related changes in the tibialis anterior muscle proteome of the mdx mouse model of dystrophinopathy. J Biomed Biotechnol 2012: 691641. doi.org/10.1155/2012/691641
Cataldi M P, Blaeser A, Lu P, Leroy V, Lu Q L (2020) ISPD Overexpression Enhances Ribitol-Induced Glycosylation of alpha-Dystroglycan in Dystrophic FKRP Mutant Mice. Mol Ther Methods Clin Dev 17: 271-80. doi.org/10.1016/j.omtm2019.12.005
Cataldi M P, Lu P, Blaeser A, Lu Q L (2018) Ribitol restores functionally glycosylated alpha-dystroglycan and improves muscle function in dystrophic FKRP-mutant mice. Nat Commun 9: 3448. doi.org/10.1038/s41467-018-05990-z
Chandel I, Campbell K P (2023) Identification of Matriglycan by Dual Exoglycosidase Digestion of alpha-Dystroglycan. Bio Protoc 13: e4827. doi.org/10.21769/BioProtoc.4827
Charleston J S, Schnell F J, Dworzak J, Donoghue C, Lewis S, Chen L, Young G D, Milici A J, Voss J, Dealwis U, Wentworth B, Rodino-Klapac L R, Sahenk Z, Frank D, Mendell J R (2018) Eteplirsen treatment for Duchenne muscular dystrophy: Exon skipping and dystrophin production. Neurology 90: e2146-e54. doi.org/10.1212/WNL.0000000000005680
Chen Y, Zhou Z, Yang W, Bi N, Xu J, He J, Zhang R, Wang L, Abliz Z (2017) Development of a Data-Independent Targeted Metabolomics Method for Relative Quantification Using Liquid Chromatography Coupled with Tandem Mass Spectrometry. Anal Chem 89: 6954-62. doi.org/10.1021/acs.analchem.6b04727
Crowe K E, Shao G, Flanigan K M, Martin P T (2016) N-terminal alpha Dystroglycan (alphaDG-N): A Potential Serum Biomarker for Duchenne Muscular Dystrophy. J Neuromuscul Dis 3: 247-60. doi.org/10.3233/JND-50127
Endo T (2015) Glycobiology of alpha-dystroglycan and muscular dystrophy. J Biochem 157: 1-12. doi.org/10.1093/jb/mnvu066
Ervasti J M, Campbell K P (1993) A role for the dystrophin-glycoprotein complex as a transmembrane linker between laminin and actin. J Cell Biol 122: 809-23. doi.org/10.1083/jcb.122.4.809
FDA guidance:Bioanalytical Method Validation Guidance for Industry. www.fda.gov/regulatory-information/search-fda-guidance-documents/bioanalytical-method-validation-guidance-industry.
Fritschy J M (2008) Is my antibody-staining specific? How to deal with pitfalls of immunohistochemistry. Eur J Neurosci 28: 2365-70. doi.org/10.1111/j.1460-9568.2008.06552.x
Georganopoulou D G, Moisiadis V G, Malik F A, Mohajer A, Dashevsky T M, Wuu S T, Hu C K (2021) A Journey with LGMD: From Protein Abnormalities to Patient Impact. Protein J 40: 466-88. doi.org/10.1007/s10930-021-10006-9
Gerin I, Ury B, Breloy I, Bouchet-Seraphin C, Bolsee J, Halbout M, Graff J, Vertommen D, Muccioli G G, Seta N, Cuisset J M, Dabaj I, Quijano-Roy S, Grahn A, Van Schaftingen E, Bommer G T (2016) ISPD produces CDP-ribitol used by FKTN and FKRP to transfer ribitol phosphate onto alpha-dystroglycan. Nat Commun 7: 11534. doi.org/10.1038/ncomms11534
Godfrey C, Clement E, Mein R, Brockington M, Smith J, Talim B, Straub V, Robb S, Quinlivan R, Feng L, Jimenez-Mallebrera C, Mercuri E, Manzur A Y, Kinali M, Torelli S, Brown S C, Sewry C A, Bushby K, Topaloglu H, North K, Abbs S, Muntoni F (2007) Refining genotype phenotype correlations in muscular dystrophies with defective glycosylation of dystroglycan. Brain 130: 2725-35. doi.org/10.1093/brain/awm212
Han R, Kanagawa M, Yoshida-Moriguchi T, Rader E P, Ng R A, Michele D E, Muirhead D E, Kunz S, Moore S A, Iannaccone S T, Miyake K, Mcneil P L, Mayer U, Oldstone M B, Faulkner J A, Campbell K P (2009) Basal lamina strengthens cell membrane integrity via the laminin G domain-binding motif of alpha-dystroglycan. Proc Natl Acad Sci USA 106: 12573-9. doi.org/10.1073/pnas.0906545106
Harrison R, Hitchen P G, Panico M, Morris H R, Mekhaiel D, Pleass R J, Dell A, Hewitt J E, Haslam S M (2012) Glycoproteomic characterization of recombinant mouse alpha-dystroglycan. Glycobiology 22: 662-75. doi.org/10.1093/glycob/cws002
Heidebrecht F, Heidebrecht A, Schulz I, Behrens S E, Bader A (2009) Improved semiquantitative Western blot technique with increased quantification range. J Immunol Methods 345: 40-8. doi.org/10.1016/j.jim.2009.03.018
Hewitt J E (2009) Abnormal glycosylation of dystroglycan in human genetic disease. Biochim Biophys Acta 1792: 853-61. doi.org/10.1016/j.bbadis.2009.06.003
Iachettini S, Valaperta R, Marchesi A, Perfetti A, Cuomo G, Fossati B, Vaienti L, Costa E, Meola G, Cardani R (2015) Tibialis anterior muscle needle biopsy and sensitive biomolecular methods:
a useful tool in myotonic dystrophy type 1. Eur J Histochem 59: 2562. doi.org/10.4081/ejh.2015.2562
Inamori K, Yoshida-Moriguchi T, Hara Y, Anderson M E, Yu L, Campbell K P (2012) Dystroglycan function requires xylosyl- and glucuronyltransferase activities of LARGE. Science 335: 93-6. doi.org/10.1126/science.1214115
Jimenez-Mallebrera C, Torelli S, Feng L, Kim J, Godfrey C, Clement E, Mein R, Abbs S, Brown S C, Campbell K P, Kroger S, Talim B, Topaloglu H, Quinlivan R, Roper H, Childs A M, Kinali M, Sewry C A, Muntoni F (2009) A comparative study of alpha-dystroglycan glycosylation in dystroglycanopathies suggests that the hypoglycosylation of alpha-dystroglycan does not consistently correlate with clinical severity. Brain Pathol 19: 596-611. doi.org/10.1111/j.1750-3639.2008.00198.x
Johnson E K, Li B, Yoon J H, Flanigan K M, Martin P T, Ervasti J, Montanaro F (2013) Identification of new dystroglycan complexes in skeletal muscle. PLoS One 8: e73224. doi.org/10.1371/journal.pone.0073224
Johnson K, Bertoli M, Phillips L, Topf A, Van Den Bergh P, Vissing J, Witting N, Nafissi S, Jamal-Omidi S, Lusakowska A, Kostera-Pruszczyk A, Potulska-Chromik A, Deconinck N, Wallgren-Pettersson C, Strang-Karlsson S, Colomer J, Claeys K G, De Ridder W, Baets J, Von Der Hagen M, Fernandez-Torron R, Zulaica Ijurco M, Espinal Valencia J B, Hahn A, Durmus H, Willis T, Xu L, Valkanas E, Mullen T E, Lek M, Macarthur D G, Straub V (2018) Detection of variants in dystroglycanopathy-associated genes through the application of targeted whole-exome sequencing analysis to a large cohort of patients with unexplained limb-girdle muscle weakness. Skelet Muscle 8: 23. doi.org/10.1186/s13395-018-0170-1
Joyce N C, Oskarsson B, Jin L W (2012) Muscle biopsy evaluation in neuromuscular disorders. Phys Med Rehabil Clin N Am 23: 609-31. doi.org/10.1016/j.pmr.2012.06.006
Kanagawa M, Kobayashi K, Tajiri M, Manya H, Kuga A, Yamaguchi Y, Akasaka-Manya K, Furukawa J I, Mizuno M, Kawakami H, Shinohara Y, Wada Y, Endo T, Toda T (2016) Identification of a Post-translational Modification with Ribitol-Phosphate and Its Defect in Muscular Dystrophy. Cell Rep 14: 2209-23. doi.org/10.1016/j.celrep.2016.02.017
Kanagawa M, Saito F, Kunz S, Yoshida-Moriguchi T, Barresi R, Kobayashi Y M, Muschler J, Dumanski J P, Michele D E, Oldstone M B, Campbell K P (2004) Molecular recognition by LARGE is essential for expression of functional dystroglycan. Cell 117: 953-64. doi.org/10.1016/j.cell.2004.06.003
Le S, Yu M, Hovan L, Zhao Z, Ervasti J, Yan J (2018) Dystrophin As a Molecular Shock Absorber. ACS Nano 12: 12140-48. doi.org/10.1021/acsnano.8b05721
Lee A J, Jones K A, Butterfield R J, Cox M O, Konersman C G, Grosmann C, Abdenur J E, Boyer M, Beson B, Wang C, Dowling J J, Gibbons M A, Ballard A, Janas J S, Leshner R T, Donkervoort S, Bonnemann C G, Malicki D M, Weiss R B, Moore S A, Mathews K D (2019) Clinical, genetic, and pathologic characterization of FKRP Mexican founder mutation c.1387A>G. Neurol Genet 5: e315. doi.org/10.1212/NXG.000000000000315
Liang Y, Hao H, Kang A, Xie L, Xie T, Zheng X, Dai C, Wan L, Sheng L, Wang G (2010) Qualitative and quantitative determination of complicated herbal components by liquid chromatography hybrid ion trap time-of-flight mass spectrometry and a relative exposure approach to herbal pharmacokinetics independent of standards. J Chromatogr A 1217: 4971-9. doi.org/10.1016/j.chroma.2010.05.056
Martin P T (2005) The dystroglycanopathies: the new disorders of O-linked glycosylation. Semin Pediatr Neurol 12: 152-8. doi.org/10.1016/j.spen.2005.10.003
Mercuri E, Brockington M, Straub V, Quijano-Roy S, Yuva Y, Herrmann R, Brown S C, Torelli S, Dubowitz V, Blake D J, Romero N B, Estournet B, Sewry C A, Guicheney P, Voit T, Muntoni F (2003) Phenotypic spectrum associated with mutations in the fukutin-related protein gene. Ann Neurol 53: 537-42. doi.org/10.1002/ana.10559)
Michele D E, Barresi R, Kanagawa M, Saito F, Cohn R D, Satz J S, Dollar J, Nishino I, Kelley R I, Somer H, Straub V, Mathews K D, Moore S A, Campbell K P (2002) Post-translational disruption of dystroglycan-ligand interactions in congenital muscular dystrophies. Nature 418: 417-22. doi.org/10.1038/nature00837
Miskiewicz E I, Macphee D J (2019) Lysis Buffer Choices Are Key Considerations to Ensure Effective Sample Solubilization for Protein Electrophoresis. Methods Mol Biol 1855: 61-72. doi.org/10.1007/978-1-4939-8793-1_5
Miyara M, Umeda K, Ishida K, Sanoh S, Kotake Y, Ohta S (2016) Protein extracts from cultured cells contain nonspecific serum albumin. Biosci Biotechnol Biochem 80: 1164-7. doi.org/10.1080/09168451.2016.1151338
Muntoni F, Torelli S, Wells D J, Brown S C (2011) Muscular dystrophies due to glycosylation defects: diagnosis and therapeutic strategies. Curr Opin Neurol 24: 437-42. doi.org/10.1097/WCO.0b013e32834a95e3
Murphy A P, Straub V (2015) The Classification, Natural History and Treatment of the Limb Girdle Muscular Dystrophies. J Neuromuscul Dis 2: S7-S19. doi.org/10.3233/JND-150105
Nickolls A R, Bonnemann C G (2018) The roles of dystroglycan in the nervous system: insights from animal models of muscular dystrophy. Dis Model Mech 11.doi.org/10.1242/dmm.035931
Peach M, Marsh N, Miskiewicz E I, Macphee D J (2015) Solubilization of proteins: the importance of lysis buffer choice. Methods Mol Biol 1312: 49-60. doi.org/10.1007/978-1-4939-2694-7_8
Pillai-Kastoori L, Schutz-Geschwender A R, Harford J A (2020) A systematic approach to quantitative Western blot analysis. Anal Biochem 593: 113608. doi.org/10.1016/j.ab.2020/113608
Pitre A, Pan Y, Pruett S, Skalli O (2007) On the use of ratio standard curves to accurately quantitate relative changes in protein levels by Western blot. Anal Biochem 361: 305-7. doi.org/10.1016/j.ab 2006.11.008
Poppe M, Cree L, Bourke J, Eagle M, Anderson L V, Birchall D, Brockington M, Buddles M, Busby M, Muntoni F, Wills A, Bushby K (2003) The phenotype of limb-girdle muscular dystrophy type 21. Neurology 60: 1246-51. doi.org/10.1212/01.wnl.0000058902.88181.3d
Rader E P, Turk R, Willer T, Beltran D, Inamori K, Peterson T A, Engle J, Prouty S, Matsumura K, Saito F, Anderson M E, Campbell K P (2016) Role of dystroglycan in limiting contraction-induced injury to the sarcomeric cytoskeleton of mature skeletal muscle. Proc Natl Acad Sci USA 113: 10992-7. doi.org/10.1073/pnas.1605265113
Schnell F, Frank D, Fletcher S, Johnsen R D, Wilton S D (2019) Challenges of Interpreting Dystrophin Content by Western Blot. U S Neurology 15:40. doi.org/10.17925/USN.2019.15.1.40
Schutz Geschwender A, Zhang Y, Holt T, Mcdermitt D, Olive, D M (2004) Quantitative, Two-Color Western Blot Detection With Infrared Fluorescence. LI-COR Biosciences
Sheikh M O, Capicciotti C J, Liu L, Praissman J, Ding D, Mead D G, Brindley M A, Willer T, Campbell K P, Moremen K W, Wells L, Boons G (2022) Cell surface glycan engineering reveals that matriglycan alone can recapitulate dystroglycan binding and function. Nature Communications 13. doi.org/10.1038/s41467-022-31205-7
Stevens E, Carss K J, Cirak S, Foley A R, Torelli S, Willer T, Tambunan D E, Yau S, Brodd L, Sewry C A, Feng L, Haliloglu G, Orhan D, Dobyns W B, Enns G M, Manning M, Krause A, Salih M A, Walsh C A, Hurles M, Campbell K P, Manzini M C, Consortium U K, Stemple D, Lin Y Y, Muntoni F (2013) Mutations in B3GALNT2 cause congenital muscular dystrophy and hypoglycosylation of alpha-dystroglycan. Am J Hum Genet 92: 354-65. doi.org/10.1016/j.ajhg.2013.01.016
Straub V, Murphy A, Udd B, Group LWS (2018) 229th ENMC international workshop: Limb girdle muscular dystrophies—Nomenclature and reformed classification Naarden, the Netherlands, 17-19 Mar. 2017. Neuromuscul Disord 28: 702-10. doi.org/10.1016/j.nmd.2018.05.007
Sun L, Konstantinidi A, Ye Z, Nason R, Zhang Y, Bull C, Kahl-Knutson B, Hansen L, Leffler H, Vakhrushev S Y, Yang Z, Clausen H, Narimatsu Y (2022) Installation of O-glycan sulfation capacities in human HEK293 cells for display of sulfated mucins. J Biol Chem 298: 101382. doi.org/10.016/j.jbc.2021.101382
Suzuki O, Koura M, Noguchi Y, Uchio-Yamada K, Matsuda J (2011) Use of sample mixtures for standard curve creation in quantitative western blots. Exp Anim 60: 193-6. doi.org/10.1538/expanim.60.193
Taylor S C, Berkelman T, Yadav G, Hammond M (2013) A defined methodology for reliable quantification of Western blot data. Mol Biotechnol 55: 217-26. doi.org/10.1007/s12033-013-9672-6
Willis E, Moore S A, Cox M O, Stefans V, Aravindhan A, Gokden M, Veerapandiyan A (2022) Limb-Girdle Muscular Dystrophy R9 due to a Novel Complex Insertion/Duplication Variant in FKRP Gene. Child Neurol Open 9: 2329048X221097518. doi.org/10.1177/2329048X221097518
Wu B, Drains M, Shah S N, Lu P J, Leroy V, Killilee J, Rawls R, Tucker J D, Blaeser A, Lu Q L (2022) Ribitol dose-dependently enhances matriglycan expression and improves muscle function with prolonged life span in limb girdle muscular dystrophy 21 mouse model. PLoS One 17: e0278482. doi.org/10.1371/journal.pone.0278482
Yoshida-Moriguchi T, Campbell K P (2015) Matriglycan: a novel polysaccharide that links dystroglycan to the basement membrane. Glycobiology 25: 702-13. doi.org/10.1093/glycob/cwv021
Zardini E, Franciotta D, Melzi D'eril G V (1993) Detection of dystrophin with a modified western blot technique in muscle tissue extracts. Clin Chem 39: 915. www.ncbi.nlm.nih.gov/pubmed/8485897

1. A method, comprising:
a) determining a difference between a first amount of a glycosylated form of alpha-dystroglycan (αDG) in a first sample taken from a subject undergoing treatment for a dystroglycanopathy at a first time and a second amount of αDG in a second sample taken from the subject at a second time; and
b) based at least in part on a),
i) determining that the treatment should be continued,
ii) determining that the treatment should be discontinued, or
iii) determining that the treatment should be adjusted.

2. A method of evaluating a subject undergoing treatment for a dystroglycanopathy, comprising:
a) providing a first sample containing a first amount of a glycosylated form of alpha-dystroglycan (αDG) from a subject, wherein the first sample is taken from the subject at a first time;
b) providing a second sample containing a second amount of a glycosylated form of αDG from the subject, wherein the second sample is taken from the subject at a second time later than the first time;
c) determining the first amount in the first sample;
d) determining the second amount in the second sample; and
e) determining a difference between the first amount and the second amount.

3. (canceled)

4. (canceled)

5. The method of claim 2, further comprising f) based at least in part on the difference determined in e), i) determining that the treatment should be continued, ii) determining that the treatment should be discontinued, or iii) determining that the treatment should be increased or decreased.

6. (canceled)

7. (canceled)

8. The method of claim 2, wherein the first time is prior to commencement of the treatment for the dystroglycanopathy, and the second time is during or after the treatment of the dystroglycanopathy.

9. (canceled)

10. The method of claim 8, wherein the second time is at least about 3 months, 6 months, 9 months, or 12 months after the first time.

11-13. (canceled)

14. The method of claim 2, wherein the first sample and the second sample are tissue biopsy samples.

15. The method of claim 14, wherein the first sample and the second sample are tibialis anterior (TA) samples.

16. The method of claim 2, wherein determining the difference between the first amount and the second amount comprises performing a Western blotting analysis.

17. The method of claim 16, wherein the first amount and the second amount are determined by integrating a signal over a range of interest and interpolating the signal to a standard curve, wherein the standard curve is based on amounts of glycosylated αDG measured for healthy subjects not undergoing treatment for a dystroglycanopathy, and wherein the interpolation is performed using regression to a quadratic equation.

18. (canceled)

19. (canceled)

20. The method of claim 2, wherein determining the first amount and the second amount comprise contacting the first sample and the second sample with one or more antibodies.

21. The method of claim 20, wherein an antibody of the one or more antibodies is used to determine an amount of the glycosylated form of αDG having a molecular weight in the range of interest of between about 125 kiloDaltons (kDa) and about 260 kDa.

22. The method of claim 20, wherein the one or more antibodies are selected from AF6868 alpha-dystroglycan, IIH6C4 alpha-dystroglycan, IR800CW Mouse Anti-Sheep, and IR680 Goat Anti-Mouse antibodies.

23. The method of claim 2, wherein determining the first amount and the second amount comprises detection of a fluorescent signal at about 700 nanometers (nm) and/or 800 nm.

24. The method of claim 2, wherein the dystroglycanopathy is selected from Walker-Warburg Syndrome, Muscle Eye Brain Disease, Fukuyama Congenital Muscular Dystrophy (CMD), or a Limb Girdle Muscular Dystrophy.

25. The method of claim 24, wherein the dystroglycanopathy is limb girdle muscular dystrophy type 2i (LGMD2I/R9), LGMD type 2m (LGMD2m), or LGMD type 2u (LGMD2u).

26. (canceled)

27. The method of claim 2, wherein the dystroglycanopathy is associated with a defect in fukutin (FKTN), Fukutin-related protein (FKRP), or isoprenoid synthase domain-containing protein (ISPD).

28. The method of claim 2, wherein the treatment for the dystroglycanopathy comprises administration of a therapeutically effective amount of ribitol.

29-31. (canceled)

32. The method of claim 2, wherein the treatment comprises administering 0.5 grams of ribitol per day (g/day), 1 g/day, 1.5 g/day, 2 g/day, 3 g/day, 4 g/day, 5 g/day, 6 g/day, 7.5 g/day, 10 g/day, 12 g/day, 12.5 g/day, 15 g/day, 20 g/day, 25 g/day, 30 g/day, 35 g/day, 40 g/day, 45 g/day, 50 g/day, 55 g/day, or 60 g/day.

33. The method of claim 2, wherein the treatment comprises administering ribitol at a dose effective to achieve an area under the concentration-time curve (AUC0-24) steady state level of between 100 (μg·h)/mL and 8000 (μg·h)/m.

34. The method of claim 2, wherein ribitol is administered once, twice, three times, or four times daily.

35. The method of claim 2, further comprising evaluating the subject using a North Star Assessment for Limb Girdle Type Muscular Dystrophies (NSAD), Performance of upper limb test 2.0 (PUL2.0), 10-meter walk test (10MWT), 100-meter timed test (100 MTT), forced vital capacity (FVC) assessment, serum creatine kinase (CK) levels, or a combination thereof.

36-39. (canceled)

40. The method of claim 2, further comprising determining (i) a first ratio of the glycosylated form of αDG to the total αDG in the first sample and (ii) a second ratio of the glycosylated form of αDG to the total αDG in the second sample.
